(19) United States
(12) Patent Application Publication
(10) Pub. No.: US 2021/0345995 A1
(43) Pub. Date: Nov. 11, 2021

(54) AUTOMATED SAMPLE SCANNING AND SEGREGATION SYSTEM AND METHODS

(71) Applicant: DermTech, Inc., La Jolla, CA (US)

(72) Inventors: John Daniel DOBAK, III, La Jolla, CA (US); James ROCK, La Jolla, CA (US); Zuxu YAO, San Diego, CA (US)

(21) Appl. No.: 17/315,199

(22) Filed: May 7, 2021

Related U.S. Application Data
(60) Provisional application No. 63/022,364, filed on May 8, 2020.

(51) Int. Cl. A61B 10/02; G01N 1/28

(52) U.S. Cl. CPC A61B 10/02; G01N 2001/2873; G01N 1/286

(57) ABSTRACT

Provided are systems and methods for automated scanning and cutting of cells of interest from a tissue sample collector.

### CROSS-REFERENCE TO RELATED APPLICATIONS

This application claims the benefit of U.S. Provisional Application No. 63/022,364 filed May 8, 2020, which application is incorporated herein by reference for all purposes in its entirety.

### BACKGROUND

Separation of lesion-derived cells from non-lesion surrounding area-derived cells may be cut in an automated fashion to improve the throughput and quality of molecular/genomic analysis of the lesion from which the cell samples were derived. In some embodiments, the cell samples are collected via a non-invasive adhesive patches which may be cut using in an automated fashion using the systems and methods described herein.

### SUMMARY

Collected tissues samples may include a collection of cells with differentiated value. In some embodiments, inclusion of cells outside of an area of interest may negatively affect results of sample analysis. In some embodiments, collection of tissue samples via adhesive tape is performed using an adhesive patch that is often sized larger than the desired collection area (e.g., lesion, mole). The larger sizing of the adhesive patch is designed in consideration of different lesion sizes, as well as the ergonomics and coordination required to use adhesive tape-stripping methods since not all lesions are alike, nor is the ease of sampling the same.

As one example, a non-invasive tissue collector may be an adhesive patch designed for collecting epidermal lesional skin tissue from patients with their skin cancers in early stages (e.g., melanoma), which may appear as small lesions, often smaller than the size of the patch collector. Accordingly, the adhesive patch may collect a combination of cells derived directly from the lesion as well as cells derived from the surrounding non-lesion area. Prior to analysis of underlying gene expression profiles, only cells from the lesion area may be desirable to be used to extract nucleic acids, and the cells of the surrounding area should be separated and removed because genomic material obtained from the cells collected from the surrounding area can dilute samples and interfere with the accuracy of the results obtained from the cells collected from the lesion. Blending or mixing up cells from lesion and non-lesional skins may dilute the molecular signals from the lesional skin cells and reduce test performance (e.g., causing false negative detection), especially when the lesion is small for earlier stages of cancer (e.g., where the molecular signals from lesional skin cells would be much more diluted by cells from the surrounding non-lesional skin). Unfortunately, these different areas may not be easily differentiated on the tissue collector after sampling because the cells obtained from both areas look similar by visual inspection. In one example, in order to delineate these areas, the edges of the lesion may be outlined on a clear adhesive patch so that the lesion area cells can be visually differentiated from the surrounding area cells. This outlined delineation can be used in the lab to remove the surrounding sample area of the adhesive patch from the lesion area in order to improve the analysis of the lesional tissue containing cells that migrated up from deeper layers of the lesion of interest.

Systems and methods described herein provide an automated solution for scanning and cutting portions of a sample with cells of interest on a non-invasive tissue sampling test kit for high throughput analysis that isolates the genomic information from the samples for highest sensitivity and specificity. In some embodiments, the non-invasive tissue sampling test kit includes one or more adhesive patches that collects cells of interest by adhesion to an adhesive patch. In one embodiment, an array of tissue sampling kits, with adhesive patches and cell samples adhered thereto and lesion area delineated thereon, are aligned on a platform, each lesion area scanned by a computer, after which a laser or other automatable technology cuts around the delineated lesion areas based on appropriately programmed computer software.

In some embodiments, provided herein are methods for automated scanning and cutting of cells of interest from a tissue sample collection kit, the method comprising: receiving the tissue sample collection kit comprising at least one sample collector, the at least one sample collector comprising the cells of interest; placing the at least one sample collector on a platform; scanning the at least one sample collector; identifying a delineation of the cells of interest on each sample collector; and cutting the cells of interest from a remaining portion of each sample collector with an automated cutting system based on the delineation. In some embodiments, the method further comprises a step of analyzing the cells of interest cut from the remaining portion of the sample collector. In some embodiments, the sample collection kit comprises a non-invasive adhesive sampling process. In some embodiments, the step of placing the at least one sample collector on the platform further comprises placing a plurality of sample collectors on the platform to form an array of sample collectors. In some embodiments, each sample collector comprises an adhesive matrix, wherein the cells of interest are collected from a skin area of a patient by adhesion to the adhesive matrix.

In some embodiments, the sample collection kit comprises a means for patient identification. In some embodiments, identifying the delineation of the cells of interest further comprises delineating the cells of interest obtained from a lesion area from other cells obtained from a non-lesion surrounding area. In some embodiments, delineating the cells of interest obtained from a lesion area from other cells obtained from a non-lesion surrounding area includes receiving data obtained during a non-invasive adhesion sampling process. In some embodiments, the steps of scanning the array and identifying the delineation are performed by a computer system.

In some embodiments, the delineation is based on a disease indication, a disease progression state, a type of lesion, a size of lesion, or a combination thereof. In some embodiments, the method further comprises a step of validating each sample collector. In some embodiments, validating the sample collector comprises scanning the sample collector for inconsistencies, a presence of interfering substances, or a combination thereof.

In some embodiments, the step of cutting the cells of interest further comprises cutting a lesion area of the sample collector from a non-lesion surrounding area. In some embodiments, the automated cutting system comprises a mechanical cutting system, a plasma cutting system, or a laser cutting system. In some embodiments, step of cutting the cells of interest is performed based on identified markings on each of the one or more sample collectors obtained from a computerized scanning of the array of sample collectors.

In some embodiments, provided herein are systems for automated scanning and cutting of cells of interest from a tissue sample collection kit comprising: a platform; one or more imaging devices directed toward the platform; a computer-implemented software program configured to identify a delineation between a first area (e.g., the cells of interest) and a second area (e.g., surrounding portion) of a sample collector of the tissue sample collection kit using the one or more imaging devices.

In some embodiments, the system further comprises a cutting mechanism, wherein the cutting mechanism cuts the sample collector at a boundary formed by the delineation between the cells of interest and a surrounding portion of the sample collector. In some embodiments, the cutting mechanism is guided by the computer-implemented software program. In some embodiments, the cutting mechanism comprises a mechanical cutting system, a plasma cutting system, or a laser cutting system. In some embodiments, the platform is configured to support an array formed by a plurality of sample collectors of the sample collection kit. In some embodiments, sample collection kit comprises a plurality of non-invasive sample collectors.

In some embodiments, provided herein is a method for isolating cells of interest from a tissue sample collection kit, the method comprising: receiving one or more sample collectors comprising cells of interest; positioning the one or more sample collectors on a substrate; imaging the one or more sample collectors to generate at least one first image; applying a software algorithm to the at least one first image to identify a delineation between the cells of interest and a surrounding portion of each sample collector; and cutting the cells of interest from a remaining portion of each sample collector with a cutting system based on the identified delineation.

In some embodiments, the one or more sample collectors comprises one or more non-invasive, adhesive sample collectors. In some embodiments, the tissue comprises skin tissue, and wherein the cells of interest comprise skin cells. In some embodiments, the method further comprises receiving one or more images captured with a mobile teledermatology application, wherein the one or more images captured with the mobile teledermatology application show a substantially clear, non-invasive, adhesive sample collector positioned on the skin of an individual, and wherein a skin condition from which the cells of interest are derived is visible though the substantially clear, non-invasive, adhesive sample collector.

In some embodiments, the one or more sample collectors comprises a plurality of sample collectors arranged in an array of sample collectors. In some embodiments, the array of sample collectors is associated with at least one fiducial marker. In some embodiments, the substrate comprises one or more positioning cut-outs configured to align the one or more sample collectors. In some embodiments, the method further comprises applying sheet cover on top of the one or more sample collectors positioned on the substrate. In some embodiments, imaging the one or more sample collectors to generate at least one first image is performed by an optical scanning system.

In some embodiments, the method further comprises validating the identified delineation by human observation, or measurement, or both. In some embodiments, the identified delineation comprises digital information comprising a plurality of points and one or more lines and/or one or more curves connecting the points to form an open or closed polygon. In some embodiments, the method further comprises editing the identified delineation. In some embodiments, editing the identified delineation comprises editing one or more points, one or more lines, or one or more curves. In some embodiments, identifying the delineation between the cells of interest and a surrounding portion of a sample collector comprises delineating the cells of interest obtained from a lesion area from other cells obtained from a non-lesion surrounding area.

In some embodiments, the software algorithm comprises a computer vision algorithm. In some embodiments, the computer vision algorithm comprises an edge detection algorithm. In some embodiments, wherein imaging the one or more sample collectors to generate at least one first image comprises imaging both sides of the one or more sample collectors. In some embodiments, the imaging captures a unique identifier on one side of the one or more sample collectors and information pertaining to the cells of interest on the other side of the one or more sample collectors.

In some embodiments, the cutting is performed with a mechanical cutting system, a plasma cutting system, or a laser cutting system. In some embodiments, the cutting is performed with a laser cutting system. In some embodiments, the method further comprises imaging the one or more sample collectors, subsequent to the cutting, to generate at least one second image. In some embodiments, imaging the one or more sample collectors, subsequent to the cutting, to generate at least one second image comprises imaging both sides of the one or more sample collectors. In some embodiments, the steps imaging the one or more sample collectors to generate at least one first image; applying a software algorithm to the at least one first image to identify a delineation between the cells of interest and a surrounding portion of each sample collector; and cutting the cells of interest from a remaining portion of each sample collector with a cutting system based on the identified delineation are performed by an automated, computer-controlled apparatus. In some embodiments, the automated apparatus has a throughput of at least 16, at least 32, or at least 48 sample collectors per minute.

In some embodiments, the method further comprises providing the tissue sample collection kit to an individual in need thereof. In some embodiments, the method further comprises analyzing the cells of interest.

In some embodiments, provided herein is a system for isolating cells of interest from a tissue sample collection kit, the system comprising: an imaging apparatus; a cutting apparatus; and a computing device comprising at least one processor, a communications interface, and instructions executable by the last least one processor to provide an application; wherein the computing device is communicatively couple to the imaging apparatus and the cutting apparatus through the communications interface; wherein the application is configured to perform operations comprising: instructing the imaging apparatus to image one or more sample collectors to generate at least one first image; applying a software algorithm to the at least one first image to identify a delineation between the cells of interest and a surrounding portion of each sample collector; and instructing the cutting apparatus to cut the cells of interest from a remaining portion of each sample collector with a cutting system based on the identified delineation.

In some embodiments, the imaging apparatus comprises an optical scanner. In some embodiments, the cutting apparatus comprises a mechanical cutting system, a plasma cutting system, or a laser cutting system. In some embodiments, the cutting apparatus comprises a laser cutting system. In some embodiments, the imaging apparatus and the cutting apparatus are implemented as distinct devices. In some embodiments, the imaging apparatus and the cutting apparatus each comprise a platform configured to receive and secure one or more sample collectors. In some embodiments, the one or more sample collectors comprises one or more non-invasive, adhesive sample collectors. In some embodiments, the tissue comprises skin tissue, and wherein the cells of interest comprise skin cells.

In some embodiments, the imaging apparatus and the cutting apparatus are integrated into a single device. In some embodiments, the single device comprises a platform configured to receive and secure one or more sample collectors. In some embodiments, the one or more sample collectors comprises one or more non-invasive, adhesive sample collectors. In some embodiments, the tissue comprises skin tissue, and the cells of interest comprise skin cells.

In some embodiments, the application is configured to perform operations comprising: receiving one or more images captured with a mobile teledermatology application, wherein the one or more images captured with the mobile teledermatology application show a substantially clear, non-invasive, adhesive sample collector positioned on the skin of an individual, and a skin condition from which the cells of interest are derived is visible though the substantially clear, non-invasive, adhesive sample collector. In some embodiments, the one or more sample collectors comprises a plurality of sample collectors arranged in an array of sample collectors. In some embodiments, the array of sample collectors is associated with at least one fiducial marker.

In some embodiments, the application is configured to perform operations comprising: providing a user interface for a human user to validate the identified delineation by observation, or measurement, or both. In some embodiments, the identified delineation comprises digital information comprising a plurality of points and one or more lines and/or one or more curves connecting the points to form an open or closed polygon. In some embodiments, the application is configured to perform operations comprising: providing a user interface for a human user to edit the identified delineation. In some embodiments, editing the identified delineation comprises editing one or more points, one or more lines, or one or more curves. In some embodiments, identifying the delineation between the cells of interest and a surrounding portion of a sample collector comprises delineating the cells of interest obtained from a lesion area from other cells obtained from a non-lesion surrounding area.

In some embodiments, the software algorithm comprises a computer vision algorithm. In some embodiments, the computer vision algorithm comprises an edge detection algorithm.

In some embodiments, the imaging apparatus is configured to image both sides of the one or more sample collectors. In some embodiments, the application is configured to perform operations comprising: instructing the imaging apparatus to image the one or more sample collectors, subsequent to the cutting, to generate at least one second image. In some embodiments, the system has a throughput of at least 16, at least 32, or at least 48 sample collectors per minute.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C depict a system for automated sample scanning and cutting, in accordance with some embodiments;

FIGS. 2A-2F depict examples of tissue sample arrays, in accordance with some embodiments;

FIGS. 3-6 depict separation of tissue samples using some embodiments of the systems and methods herein;

FIGS. 7-14 depict an exemplary tissue sample collection kit and a method of use thereof, in accordance with some embodiments;

FIG. 15 illustrates a computer system that is programmed or otherwise configured to operate any of the systems or methods described herein;

FIG. 16 depicts a workflow of a method for scanning and segregating tissue samples, in accordance with some embodiments;

FIG. 17 depicts results of analyses performed on different regions of a tissue sample, in accordance with some embodiments;

FIG. 18 depicts results of analyses performed on different regions of a tissue sample, in accordance with some embodiments;

FIGS. 19A-19Z depict exemplary graphical user interfaces comprising a patient user experience (GUI/UX) for collecting images and information corresponding to a skin area to be submitted for evaluation;

FIGS. 20A-20G depict exemplary graphical user interfaces comprising a user experience (GUI/UX) for scanning, reviewing, and editing images of tissue sample collectors and delineations; and

FIGS. 21A-21B depict a workflow of a method for scanning and segregating tissue samples, in accordance with some embodiments.

### DETAILED DESCRIPTION

Provided herein are systems and method for automated scanning and cutting of cells of interest from a tissue sample collector.

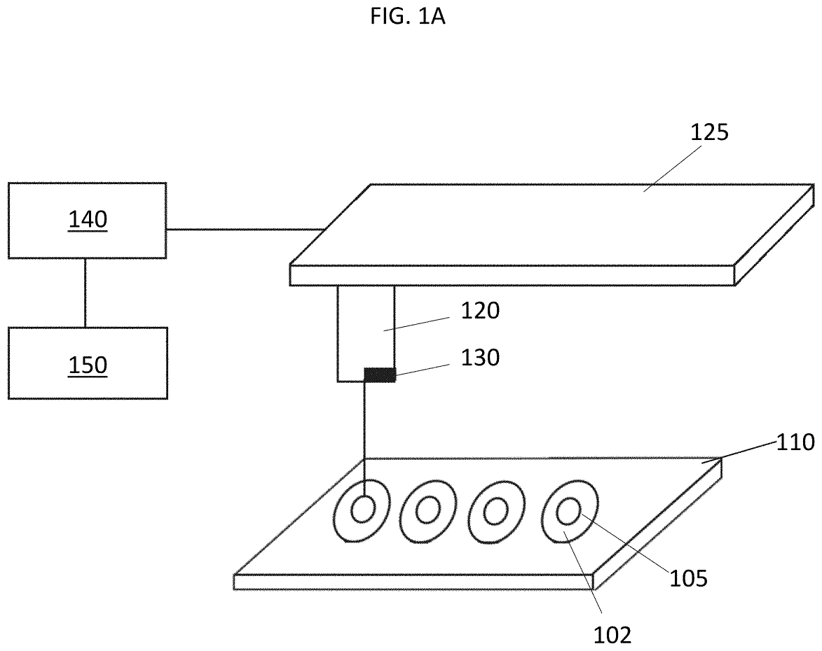

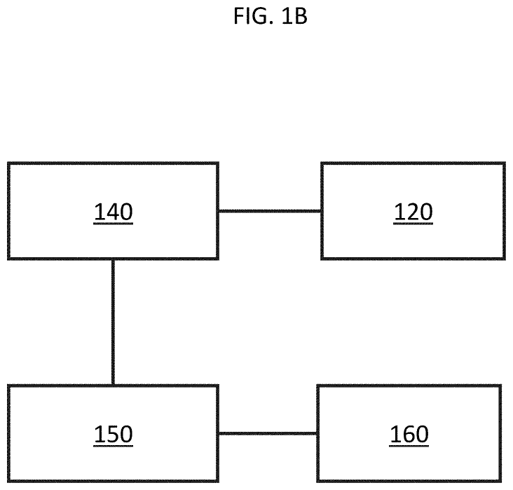

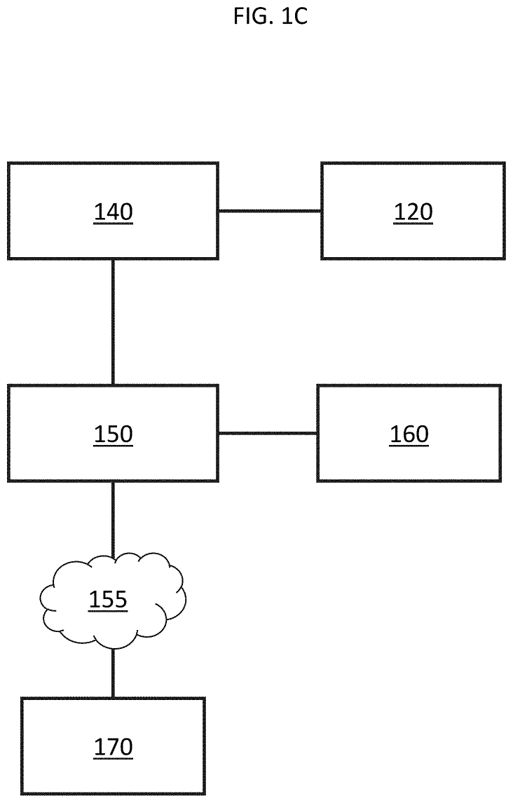

With reference to FIG. 1A, in some embodiments a system for automated scanning and cutting of cells of interest comprises a platform 110 and a cutting apparatus 120. In some embodiments, the platform 110 is configured to receive one or more sample collectors. In some embodiments, the sample collector comprises one or more adhesive tapes 102 or adhesive patches for non-invasive collection of a skin sample, as described herein.

In some embodiments, each patch of a sample collector comprises an area or portion containing cells of interest to be analyzed or evaluated. In some embodiments, the system is configured to cut along delineations formed at the boundaries 105 between a first area and a second area. In some embodiments, the first area contains the cells of interest and a second area comprises a remaining portion of the sample collector.

In some embodiments, cutting apparatus 120 comprises a mechanical cutting system, a plasma cutting system, or a laser cutting system as described herein. In some embodiments, the cutting apparatus is coupled to a cutting guide 125 to control movement of the cutting apparatus. In some embodiments, cutting guide controls movement of the cutting apparatus to cut along a delineations formed at the boundaries 105 between the first and second areas, for example the area containing the cells of interest and the remaining portion of the sample collector.

In some embodiments, the platform 110 is movable. In some embodiments, the platform moves relative to the cutting apparatus, such that the sample collector to cut along delineations formed at the boundaries 105 between the first and second areas, for example the area containing the cells of interest and the remaining portion of the sample collector. In some embodiments, both the platform 110 and cutting guide provide movement of the sample collector relative to the cutting apparatus to cut along delineations formed at the boundaries 105 between the first and second areas, for example the portion or area containing the cells of interest and the remaining portion of the sample collector.

In some embodiments, one or more image sensors 130 are provided as part of the system. In some embodiments, one or more image sensors 130 may be provided on the cutting apparatus 120. In some embodiments, the image sensors are provided at a location separate from the cutting apparatus. In some embodiments, the image sensors scan the one or more sample collectors provided on the platform to identify delineations formed between the first area and the area, for example, wherein the first area contains the cells of interest and the second area comprises a remaining portion of the sample collector. In some embodiments, cutting of the boundaries 105, formed by an identified delineation between the first and second areas of the sample collector, is based from a scan performed by the one or more image sensors 130.

In some embodiments, the system further comprises a controller 140 configured to control the movement of the cutting guide 125, platform 110, or both. The controller may be further configured to operate the cutting apparatus to perform cutting of the boundaries 105 formed by the identified delineation between the first and second areas, for example the area containing the cells of interest and the remaining portion of the sample collector.

In some embodiments, the system further comprises a computing device 150. The computing device may be configured to receive and analyzes images obtained by the one or more image sensors. In some embodiments, the computing device 150 analyzes one or more images obtained by the image sensors to identify a delineation between the first and second areas, for example the area containing the cells of interest and the remaining portion of the sample collector. In some embodiments, based on the analyzation, the computing device compiles and sends instructions to the controller 140 to provide the operation of the cutting apparatus 120 and movement by the cutting guide 125 and/or platform 110 to cut the sample collector along the boundaries 105 formed by the identified delineation between the first and second areas, for example the area containing the cells of interest and the remaining portion of the sample collector.

With reference to FIG. 1B, a system for automated scanning and cutting of tissue sample collectors is depicted. In some embodiments, the system comprises a scanning system or scanner 160 for scanning sample collectors. In some embodiments, the scanner 160 captures one or more images of the sample collectors and transmits the images to a computing device 160. In some embodiments, the computing device 150 analyzes one or more images obtained by the scanner 160 to identify a demarcation provided on the sample collector. In some embodiments, the demarcation is provided by a marker used on a surface of the sample collector. In some embodiments, based on the analyzation, the computing device compiles and sends instructions to the controller 140 to provide the operation of the cutting apparatus 120 to cut the sample collector along a boundary formed by an identified delineation representing a border of a lesion to which the sample collector was applied. In some embodiments, the computing device 150 comprises a software application which allows a user to verify and/or edit a delineation representing the border of a lesion to which the sample collector was applied.

With reference to FIG. 1C, a system for automated scanning and cutting of tissue sample collectors is depicted. In some embodiments, the system comprises a user device 170. In some embodiments, the user device 170 transmits one or more images of a sample collector adhered an area of interest on the skin of a subject. In some embodiments, the images of the sample collector adhered an area of interest on the skin of a subject are transmitted wirelessly to the computing device. In some embodiments, the images transferred from the user device to a network 155 which is accessible by the computing device 150. The area of interest may comprise a lesion or mole. In some embodiments, images provided by the user device 170 are used to verify a size or shape of the lesion.

In some embodiments, the scanner 160 captures one or more images of the sample collectors and transmits the images to a computing device 160. In some embodiments, the computing device 150 analyzes one or more images obtained by the scanner 160 to identify a demarcation provided on the sample collector. In some embodiments, images obtained by a user device 170 loaded with an image capturing and lesion mapping software application, as disclosed herein, are utilized to automatically edit a delineation representing a border of a lesion to which the sample collector was applied and on which the sample collector is cut by the cutting apparatus 120.

In some embodiments, the computing device 150 analyzes one or more images obtained by the user device 170 to identify a delineation representing a border of a lesion to which the sample collector was applied and on which the sample collector is cut by the cutting apparatus 120. In some embodiments, the computing device 150 comprises a software application which allows a user to verify and/or edit a delineation representing the border of a lesion to which the sample collector was applied, as further described herein.

### I. TISSUE SAMPLE COLLECTORS

In some embodiments, the method for automated scanning and cutting of cells of interest from a tissue sample collector comprises receiving a tissue sample collector. In some embodiments, the tissue sample collector comprises cells of interest from a non-invasive or minimally invasive sampling of the epidermis. In some embodiments, one or more tissue sample collectors are received. In some embodiments, the tissue sample collectors are components of a tissue sample collection kit.

In some embodiments, noninvasive or minimally invasive sampling devices for tissue collection comprise microneedles, blotting, tangential cutter, or other non-invasive sampling devices. In some embodiments, the non-invasive sampling device comprises an adhesive matrix, wherein the cells of interest are adhered to the adhesive matrix collected from the skin of a patient using a non-invasive adhesive sampling process.

In some embodiments, the depth at which the epidermis/cell sample is collected is 500, 400, 300, 200, 100, 90, 80, 70, 60, 50, 40, 30, 20, 10, 5, 4, 3, 2, 1 microns or less, including increments therein.

Samples collected by a tissue/sample collector kit may comprise samples from a plurality of individual cell sample areas. In some embodiments, sample collectors of a sample collection kit comprise cells of interest provided by a plurality of individual cell sample areas. In some embodiments wherein an adhesive sample collector is used, a sample collector comprises four distinct adhesive patches.

In some embodiments, the tissue collector kit further comprises identifying means for traceability and patient identification. In some embodiments, information for traceability and patient identification include indicia that identifies patient name, patient date of birth, name of the genomic test to be performed, medical record number, ordering physician, ICD10 (International Classification of Diseases) diagnostic code, and the date of sample collection. In some embodiments, each sample collector of the sample collection kit comprises information regarding the sample collector, such as indicia that identifies patient name, patient date of birth, name of the genomic test to be performed, medical record number, ordering physician, ICD10 diagnostic code, and the date of sample collection.

A. Adhesive Tape Sample Collection

In some embodiments, an adhesive skin sample collection kit for use with tape stripping methods is provided as a non-invasive means to collect skin samples with minimal discomfort. Devices described herein for skin collection in some instances are referred to as collectors, tapes, strips, stickers, or patches. Cellular material may be isolated from the skin sample and can be utilized in tests that can determine the stage of disease, the risk of disease progression and a patient's likelihood of responding to a particular treatment. Treatments may include drug therapies and biopsy. Skin sample cellular materials may include nucleic acids, polypeptides, lipids, carbohydrates and small molecules. Nucleic acids include DNA and RNA.

In some embodiments, the skin sample collected using the tape stripping method is used in combination with other clinical assays including immunohistochemistry, immunophenotyping, fluorescent in situ hybridization (FISH), and/or any combination thereof. The skin sample may not necessarily need to be removed from the adhesive tape to prove useful as an assay component. Cellular material from the skin samples may be detected from the surface of the adhesive tape matrix. Detection methods may include the use of probes configured to bind to cellular material adhered to the adhesive tape matrix. Probes may include, but are not limited to, primers configured to bind to nucleic acids, and antibodies configured to bind to polypeptides, nucleic acids, small molecules, lipids, and/or carbohydrates.

In some embodiments, the tape stripping method is part of the work up for a variety of suspected skin conditions including, but not limited to, lupus, rubeola, acne, hemangioma, psoriasis, eczema, candidiasis, impetigo, shingles, leprosy and Chron's disease. Skin conditions may also include inflammatory dermatoses, bullous diseases, infections and cancers. Skin cancers may include, but are not limited to, basal cell carcinoma, actinic keratoses, Merkel cell carcinoma, sebaceous carcinoma, squamous cell carcinoma, melanoma and dermatofibrosarcoma protuberans.

In some embodiments, the tape stripping method is performed using a plurality of adhesive tapes. Between 1 and 8 adhesive tapes may be sequentially applied and removed to collect a skin sample. The number of adhesive tapes used per skin sample may include, but is not limited to, 12, 11, 10, 9, 8, 7, 6, 5, 4, 3, 2, 1, from about 2 to about 7, from about 3 to about 6, and from about 4 to about 5. In some embodiments, an adhesive tape is applied to the skin and removed from the skin about 1 to about 8 times.

1. Components of an Adhesive Collection Kit

The adhesive tape or patch of the adhesive skin sample collection kit may comprise a first portion comprising an adhesive matrix to form a collection area and a second portion extending from the periphery of the first portion. In some embodiments, the adhesive matrix is located on a skin facing surface of the collection area. In some embodiments, the second portion may form a handling area. The handling area may comprise a tab, suitable for applying and removing the adhesive tape. The tab may be sufficient in size so that while applying the adhesive tape to a skin surface, the applicant does not come in contact with the matrix material of the first collection area. In some embodiments, the handling area does not comprise an adhesive matrix. In some embodiments, the adhesive tape does not comprise a tab. In some embodiments, the adhesive tape is handled with gloves to reduce contamination of the adhesive matrix prior to use. In some embodiments, both the collection and handling areas comprise an adhesive matrix.

In some embodiments, the collection area is a polyurethane carrier film. In some embodiments, the adhesive matrix is comprised of a synthetic rubber compound. In some embodiments, the adhesive matrix is a styrene-isoprene-styrene (SIS) linear block copolymer compound. In some embodiments, the adhesive tape does not comprise latex, silicone, or both. In some embodiments, the adhesive tape is manufactured by applying an adhesive material as a liquid-solvent mixture to the collection area and subsequently removing the solvent.

In some embodiments, the adhesive matrix comprises one or more of acrylics, silicones and hydrocarbon rubbers (like butyl rubber, styrene-butadiene rubber, ethyl-vinyl acetate polymers, styrene-isoprene-butadiene rubbers), or combination thereof. In some embodiments, tack of the adhesive matrix is measured by ASTM D1876 using XLW (EC) Auto Tensile Tester (Labthink Instrument Inc). In some embodiments, the adhesive matrix comprises a hydrophobicity of no more than 2000, 1500, 1000, 900, 800, 700, 600, 500, 400, 300, 200, or no more than 150 g/m²/24 hours. In some embodiments, hydrophobicity is measured as an upright MVTR (moisture vapor transmission rate) or inverted MVTR. In some embodiments, hydrophobicity is measured using ASTM E96-80. In some embodiments, the patch (including the adhesive matrix) comprises a hydrophobicity of no more than 2000, 1500, 1000, 900, 800, 700, 600, 500, 400, 300, 200, or no more than 150 g/m²/24 hours. In some embodiments, the adhesive matrix comprises a peel adhesion, or force exerted when removing a patch comprising the adhesive matrix. In some embodiments, peel adhesion is optimal when the desired amount of cellular material is removed from the skin, but without causing skin damage or discomfort to the patient. In some embodiments, the peel adhesion is measured using ASTM D3330. In some embodiments, the peel adhesion is 1-40, 1-30, 1-20, 5-30, 5-25, 5-20, 5-15, 3-15, 3-12, 10-20, 5-30, 15-30, or 3-10 Newtons/inch. In some embodiments, the peel adhesion is at least 1, 2, 3, 4, 5, 6, 7, 8, 9, 10, 12, 15, 20, 25, 30, or at least 35 Newtons/inch, including increments therein. In some embodiments, the peel adhesion is no more than 1, 2, 3, 4, 5, 6, 7, 8, 9, 10, 12, 15, 20, 25, 30, or no more than 35 Newtons/inch, including increments therein. In some embodiments, the peel adhesion is about 1, 2, 3, 4, 5, 6, 7, 8, 9, 10, 12, 15, 20, 25, 30, or about 35 Newtons/inch, including increments therein. In some embodiments, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 180° peel adhesion at a pull rates from about 1.0 inch/min to about 12.0 inch/min. In some embodiments, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 180° peel adhesion at a pull rates from about 4.0 inch/min to about 16.0 inch/min. In some embodiments, the adhesive matrix comprises a peel adhesion strength from about 1-40, 1-30, 1-20, 5-30, 5-25, 5-20, 5-15, 3-15, 3-12, 10-20, 5-30, 15-30, or about 3-10, as measured by ASTM D3330 at a 180° peel adhesion at a pull rates from about 0.5 inch/min to about 8 inch/min. In some embodiments, the adhesive matrix comprises a pressure sensitive adhesive. In some embodiments, the pressure sensitive adhesive exhibits a glass transition temperature lower than 20° C., 15° C., 10° C., 7° C. 6° C., 5° C., 4° C., 3° C., or lower than 2° C., including increments therein. In some embodiments, the pressure sensitive adhesive exhibits a glass transition temperature of 1-20° C., 1-15° C., 1-10° C., 1-7° C. 3-8° C., 4-6° C. or 4-10° C. In some embodiments, the pressure sensitive adhesive exhibits a glass transition temperature of about 20° C., 15° C., 10° C., 7° C., 6° C., 5° C., 4° C., 3° C., or about 2° C., including increments therein.

Adhesive patches may be transparent or opaque depending on the application. In some embodiments, the patch is opaque. In some embodiments, the patch is clear. In some embodiments, the patch has an opacity of about 1%, 2%, 5%, 8%, 10%, 15%, 20%, 25%, 30%, 40%, 50%, 60%, 70%, 80%, 90%, 95%, or about 98%. In some embodiments, the patch has an opacity of at least 1%, 2%, 5%, 8%, 10%, 15%, 20%, 25%, 30%, 40%, 50%, 60%, 70%, 80%, 90%, 95%, or at least 98%. In some embodiments, the patch has an opacity of no more than 1%, 2%, 5%, 8%, 10%, 15%, 20%, 25%, 30%, 40%, 50%, 60%, 70%, 80%, 90%, 95%, or no more than 98%. In some embodiments, the patch has an opacity after removing skin cells one or more times (peeling). In some embodiments, the patch has an opacity of about 1%, 2%, 5%, 8%, 10%, 15%, 20%, 25%, 30%, 40%, 50%, 60%, 70%, 80%, 90%, 95%, or about 98% after 1 peeling of skin cells. In some embodiments, the patch has an opacity of at least 1%, 2%, 5%, 8%, 10%, 15%, 20%, 25%, 30%, 40%, 50%, 60%, 70%, 80%, 90%, 95%, or at least 98% after 1 peeling of skin cells. In some embodiments, the patch has an opacity of no more than 1%, 2%, 5%, 8%, 10%, 15%, 20%, 25%, 30%, 40%, 50%, 60%, 70%, 80%, 90%, 95%, or no more than 98% after 1 peeling of skin cells. In some embodiments, an adhesive patch comprises a haze value of less than about 50%, 45%, 40%, 30%, 25%, 20%, 15%, 10%, or less than about 5% as measured by ASTM D1003.

Adhesive patches may comprise a matrix material. The matrix material in some embodiments is sufficiently sticky to adhere to a skin sample. The matrix material is not so sticky that is causes scarring or bleeding or is difficult to remove. In some embodiments, the matrix material is comprised of a transparent material. In some embodiments, the matrix material is biocompatible. In some embodiments, the matrix material does not leave residue on the surface of the skin after removal. In certain embodiments, the matrix material is not a skin irritant. In some embodiments, patches are applied multiple times to an area or region. In some embodiments, greater than 2 applications in the same area or region results in no more than 80, 70, 60, 50, 40, 35, 30, 25, 20, 17, 15, 12, 10, or no more than 5 g/m²/h) transepidermal water loss (TEWL), including increments therein. In some embodiments, greater than 4 applications in the same area or region results in no more than 80, 70, 60, 50, 40, 35, 30, 25, 20, 17, 15, 12, 10, or no more than 5 g/m²/h) transepidermal water loss (TEWL), including increments therein. In some embodiments, greater than 8 applications in the same area or region results in no more than 80, 70, 60, 50, 40, 35, 30, 25, 20, 17, 15, 12, 10, or no more than 5 g/m²/h) transepidermal water loss (TEWL), including increments therein. In some embodiments, greater than 6 applications in the same area or region results in no more than 80, 70, 60, 50, 40, 35, 30, 25, 20, 17, 15, 12, 10, or no more than 5 g/m²/h) transepidermal water loss (TEWL), including increments therein.

In some embodiments, an adhesive patch comprises one or more of: a backing layer, an adhesive matrix, and a non-invasive handling area. In some embodiments, a skin sample collector further comprises one or more of a release panel, individual liners, a placement area, and individual panels. In some embodiments, devices are configured for optimum peel adhesion, elasticity of the backing film, extractables, dimensions, materials, functional results, or a combination thereof. In some embodiments, the backing layer comprises a flexibility to conform to a morphology of a portion of skin comprising a lesion, and wherein the backing layer comprises a thickness such the at least one adhesive patch resists wrinkling when the at least one adhesive patch is released from the skin. In some embodiments, the at least one patch comprises a thickness such that it does not self-adhere when supported by a portion of the non-adhesive handling layer with a draft and in multiple orientations. In some embodiments, an amount of extractables and leachables released from the at least one adhesive patch is minimized to improve nucleic acid analysis. In some embodiments, the at least one adhesive patch comprises a longest dimension of about a wrinkling wavelength of the at least one adhesive patch. In some embodiments, the adhesive matrix comprises a pressure sensitive adhesive, wherein the pressure sensitive adhesive exhibits a glass transition temperatures lower than 5° C.

The adhesive patch of the adhesive skin sample collector typically comprises a backing layer. In some embodiments, the backing area comprises a first collection area comprising an adhesive matrix and a second area extending from the periphery of the first collection area. The adhesive matrix is located on a skin facing surface of the first collection area. The second area functions as a tab (or non-adhesive handling area), suitable for applying and removing the adhesive patch. The tab is sufficient in size so that while applying the adhesive patch to a skin surface, the applicant does not come in contact with the matrix material of the first collection area. In some embodiments, the adhesive patch does not contain a second area tab. In some embodiments, the adhesive patch is handled with gloves to reduce contamination of the adhesive matrix prior to use. In some embodiments, the backing comprises a soft, clear, and pliable synthetic polymer.

The backing layer may comprise any material or mixture of materials which controls rigidity or flexibility. Without being bound by theory, a backing layer enables proper conformation of the patch over the lesion of any size or shape, which leads to higher removal of cellular materials during peeling off/collection. In some embodiments, the thickness or rigidness of the backing layer is configured to prevent deformation due to static wrinkles or slip-stick patterns during peel. In some embodiments, the backing layer comprises a polyurethane carrier film. Patches described herein may comprise any number of materials which provide for the desired sampling properties (e.g., thickness, performance, patient comfort, or other property). In some embodiments, patches described herein comprise a backing layer. In some embodiments, the backing layer comprises one or more of TPU (thermoplastic polyurethane), LPDE (low density polyethylene), PET (polyethylene), PP (polypropylene), Teflon, Polyimide, PEN (Polyethylene naphthalate), PVB (polyvinyl butyral), PVOH (poly(vinyl alcohol)), PVP (Poly(vinylpolypyrrolidone)) cellulose butyrate, cellulose acetate, or a mixture thereof. In some embodiments, the backing layer comprises TPU (thermoplastic polyurethane) and LPDE (low density polyethylene). In some embodiments, the soft, clear, and pliable synthetic polymer comprises an elastomer of olefin. In some embodiments, the elastomer of olefin comprises copolymers or compounds of polymers comprising one or more of ethylene, propylene, isobutylene, vinyl acetate, vinyl alcohol, ethylene oxide, and propylene oxide. In some embodiments, the soft clear, and pliable synthetic polymer comprises a thermoplastic elastomer. In some embodiments, the thermoplastic elastomer comprises a polyester based elastomer. In some embodiments, the thermoplastic elastomer comprises a copolymer or compound of an ether or amide.

The backing layer may comprise materials or mixtures of materials selected to mitigate wrinkling of the backing layer. Wrinkling of the backing layer may be characterized by a wrinkling pattern. In some cases, the wrinkling pattern may be a regular pattern. The wrinkling pattern may be irregular. A pattern of the wrinkling may be characterized by a wrinkling wavelength (e.g., an average wavelength). The wrinkling wavelength may be a distance (e.g., an average distance) between subsequent peaks or subsequent troughs in the wrinkles. An average wavelength may be determined from an average distance between peaks for the length of the tape. Wrinkling may be static or dynamic. Static wrinkling may occur when a backing layer comprising an adhesive is adhered to a surface (e.g., a skin). Dynamic wrinkling may occur during peeling of the backing layer. In some cases, dynamic wrinkling may be caused by sticking and slipping of the backing layer during peeling. The process of peeling a backing layer comprising an adhesive may include dynamic sticking and slipping. For example, even when a user endeavors to peel a backing layer as smoothly as possible, the peeling may stop and start causing the effect of sticking and slipping. For example, during a stick, elastic potential energy may be stored in the adhesive and the bend of the tape. In some cases, both the tape and the adhesive may act like springs that store energy as they are stretched. During a slip, potential energy may be converted to kinetic energy. The sticking and slipping may occur even on microscopic length scales (e.g. length scales on the order of few microns or greater). Sticking and slipping may result in defects (e.g., wrinkles) during a peeling step. While not wishing to be bound by theory, the frequency of the stick-slip patterns in some instances decreases with the square root of the patch thickness. For example, the modulus of elasticity of the backing sheet may at least partially govern the wrinkling wavelength by the square root of the cubic root, which provides an exponent of ⅙, (i.e. λ˜Et1/6).

Parameters which effect the sticking and slipping may include elasticity of the skin, elasticity of the backing layer, strength of the adhesive, and geometric parameters such as the length and width of the tape. One or more of these parameters may affect a wavelength and frequency of wrinkling patterns in the backing layer. The skin elasticity may relate to the potential energy stored in a stick. For example, skin with a high elasticity may store greater potential energy during a stick and slip to a greater distance. The elasticity of the backing layer may relate to the potential energy stored in a stick. For example, a backing layer with a high elasticity may store greater potential energy during a stick and slip to a greater distance. The adhesive may relate to the potential energy stored in a stick. For example, a stronger adhesive may store greater potential energy during a stick and slip to a greater distance. In some cases, a separation front, the line dividing the attached portion to the separated portion, may not be a straight line during slips. For example, a slip may propagate along a width of the backing layer if the peel is along a length of the backing layer. Accordingly, a wider tape may change the wrinkling properties of the tape by changing the slip dynamics and/or by increasing the potential energy to peel per unit distance peeled along the peeling axis. In some examples, the wrinkling wavelength may be on the order of several centimeters. A wrinkling wavelength which is longer than the backing layer may mitigate dynamic wrinkles.

Static wrinkling may occur when an adhesive patch is attached to the skin. In some cases, static wrinkling may be caused by a mismatch between the extent of contraction of the soft foundation (e.g., skin) and the harder surface (e.g., the backing layer of the tape) due to the in-plane forces exerted by the adhesive. Parameters which effect the static wrinkling may include elasticity of the skin, elasticity of the backing layer, strength of the adhesive, and geometric parameters such as the length and width of the tape. One or more of these parameters may affect a wavelength and frequency of wrinkling patterns in the backing layer. The extent of contraction of the soft foundation may be related to the elasticity of the soft foundation. The extent of contraction of the harder surface may be related to the elasticity of the backing layer. A mismatch between the extents of contraction may create a deformation in the peel (e.g., a wrinkle). The deformation may be characterized by an amplitude. A mismatch between the extents of contraction may cause static wrinkles. The frequency of static wrinkles may be strongly correlated with the thickness of the backing layer. In some examples, the wrinkling wavelength may be on the order of several centimeters. A wrinkling wavelength which is longer than the backing layer may mitigate static wrinkles. In some cases, a backing layer with a thickness greater than 3 mil or above may provide a wrinkling wavelength of several centimeters.

In some embodiments, the wrinkling wavelength is configured to mitigate static and/or dynamic wrinkling. In some examples, the wrinkling wavelength may be on the order of several centimeters. A wrinkling wavelength that is longer than a length of the backing layer may mitigate wrinkling. A wrinkling wavelength that is longer than a length of a patch applied to the skin may mitigate wrinkling. The wrinkling wavelength may comprise a length which is equal to or greater than, for example and without limitation, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm.

In some embodiments, flexibility is controlled by properties of the backing layer, the adhesive matrix, or both. In some embodiments, patches are configured to adhere to atypical/3-dimensional morphologies. In some embodiments, patches comprise a conformability/flexibility to contact the morphological structure of the lesion while minimizing or avoiding wrinkling of the patch upon peel/release. In some embodiments, flexibility and the thickness of the backing layer provides for the proper conformation of the patch over the lesion of any size or shape, which leads to higher removal of skin cells during peeling off/collection. In some embodiments, flexibility is measured using ASTM D882 or ASTM D1938 methods with an XLW (EC) Auto Tensile Tester (Labthink Instrument Inc). In some embodiments, the thickness of the backing layer is no more than 7, 6, 5, 4, 3, 2.5, 2.0, 1.5, 1.25, 1, 0.8, 0.7, 0.6, 0.5, 0.3, 0.2, or no more than 0.1 mils, including increments therein. In some embodiments, the thickness of the backing layer is about 7, 6, 5, 4, 3, 2.5, 2.0, 1.5, 1.25, 1, 0.8, 0.7, 0.6, 0.5, 0.3, 0.2, or about 0.1 mils, including increments therein. In some embodiments, the thickness of the backing layer is 0.1-5, 0.1-4, 0.1-3, 0.1-2, 0.1-1, 0.5-4, 0.5-3, 1-5, 2-7, 3-5, 3-10, or 1-2 mils. In some embodiments, a backing layer comprising one or more of LDP or TPU has a thickness of at least 1, 2, 2.5, 3, 3.5, 4, 4.5, 5, 5.5, or more than 6 mils, including increments therein. In some instances, elasticity is controlled by properties of the backing layer, the adhesive matrix, or both. In some instances, patches are configured to adhere to atypical/3-dimensional morphologies. In some instances, patches comprise an elasticity to contact the morphological structure of the lesion while minimizing or avoiding wrinkling of the patch upon peel/release. The elasticity may be characterized by an elastic modulus. In some embodiments, the backing layer has an elastic modulus from about 200 to about 2,000 Psi as measured by ASTM D-882. In some embodiments, the backing layer has an elastic modulus of about 250, 500, 750, 1000, 1250, 1500, 1750, 2000, 2250, or about 2500 Psi, including increments therein. In some embodiments, the backing layer has an elastic modulus of from about 1000 to about 2000 Psi, about 500 to about 3000 Psi, about 250 to about 2000 Psi, about 400 to about 2000 Psi, about 500 to about 1500 Psi, about 750 to about 2000 Psi, about 1000 to about 3000 Psi, or about 500 to about 2500 Psi. In some embodiments, the backing layer has a tensile strength of from about 7 to about 60 MPa, about 5 to about 60 MPa, about 10 to about 60 MPa, about 20 to about 80 MPa, about 30 to about 60 MPa, about 5 to about 30 MPa, about 5 to about 20 MPa, or about 7 to about 15 MPa.

In some embodiments, the adhesive tape comprises a flexible material, enabling the tape to conform to the shape of the skin surface upon application. In some embodiments, at least the collection area is flexible. In some embodiments, the tab is plastic. In some embodiments, the adhesive tape does not contain latex, silicone, or both. In some embodiments, the adhesive tape is made of a transparent material, so that the skin sampling area of the subject is visible after application of the adhesive tape to the skin surface. The transparency may ensure that the adhesive tape is applied on the desired area of skin comprising the skin area to be sampled. In some embodiments, the adhesive tape is between about 5 and about 100 mm in length. In some embodiments, the collection area is between about 5 and about 40 mm in length. In some embodiments, the collection area is between about 10 and about 20 mm in length. In some embodiments the length of the collection area is configured to accommodate the area of the skin surface to be sampled, including, but not limited to, about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm, including increments therein. In some embodiments, the collection area is elliptical. In some embodiments, the first collection area is elliptical. Without being bound by theory the length of a patch applied to the skin is comparable to the wrinkling wavelength to avoid the wavy structure on static patch before peel. In some instances, the longest linear dimension of the patch no more than 15, 12, 10, 8, 6, 5, 4, 3, 2, or no more than 1 cm, including increments therein. In some instances, the longest linear dimension of the first collection area is no more than 15, 12, 10, 8, 6, 5, 4, 3, 2, or no more than 1 cm, including increments therein.

Parameters which effect the static wrinkling may include elasticity of the skin, elasticity of the backing layer, strength of the adhesive, and geometric parameters such as the length and width of the tape. One or more of these parameters may affect a wavelength and frequency of wrinkling patterns in the backing layer. Of these, the elasticity of the skin may not be readily controllable. For example, it may be a property of the skin to which the patch may adhere. An adhesive patch may comprise one or more of the following properties: a backing thickness greater than 3 mil, a longest dimension less than 10 cm, and a backing layer with an elastic modulus between 200 and 2000 PSI. An adhesive patch may comprise one or more of the following properties: a backing thickness greater than 3 mil, a longest dimension less than 5 cm, and a backing layer with an elastic modulus between 500 and 1500 PSI. An adhesive patch may comprise one or more of the following properties: a backing thickness greater than 3 mil, a longest dimension less than 5 cm, and a backing layer with an elastic modulus between 1000 and 2000 PSI. An adhesive patch may comprise an elastic modulus of from about 1000 to about 2000 Psi, about 500 to about 3000 Psi, about 250 to about 2000 Psi, about 400 to about 2000 Psi, about 500 to about 1500 Psi, about 750 to about 2000 Psi, about 1000 to about 3000 Psi, or about 500 to about 2500 Psi; a backing thickness greater than 3 mil; and a longest dimension less than 10 cm. An adhesive patch may comprise a longest dimension of about 19 mm, about 20 mm, about 21 mm, about 22 mm, about 23 mm, about 24 mm, about 25 mm, about 30 mm, about 35 mm, about 40 mm, about 45 mm, about 50 mm, about 55 mm, about 60 mm, about 65 mm, about 70 mm, about 75 mm, about 80 mm, about 85 mm, about 90 mm, and about 100 mm; a backing thickness greater than 3 mil; and a longest dimension less than 10 cm. An adhesive patch may comprise a backing thickness of about 3 mil, about 4 mil, about 5 mil, about 6 mil, about 7 mil, about 8 mil, about 9 mil, about 10 mil, about 20 mil, about 30 mil, about 40 mil, about 50 mil, about 60 mil, about 70 mil, about 80 mil, about 90 mil, about 100 mil, or about 125 mil; a longest dimension less than 10 cm; and a backing layer with an elastic modulus between 200 and 2000 PSI.

In some embodiments, the adhesive tape of this invention is provided on a peelable release sheet in the adhesive skin sample collection kit. In some embodiments, the adhesive tape provided on the peelable release sheet is configured to be stable at temperatures between −80° C. and 30° C. for at least 6 months, at least 1 year, at least 2 years, at least 3 years, and at least 4 years. In some embodiments, the peelable release sheet is a panel of a sample collector.

The peelable release sheet may be configured to hold a plurality of adhesive tapes, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The peelable release sheet may be configured to hold about 12 adhesive tapes. The peelable release sheet may be configured to hold about 11 adhesive tapes. The peelable release sheet may be configured to hold about 10 adhesive tapes. The peelable release sheet may be configured to hold about 9 adhesive tapes. The peelable release sheet may be configured to hold about 8 adhesive tapes. The peelable release sheet may be configured to hold about 7 adhesive tapes. The peelable release sheet may be configured to hold about 6 adhesive tapes. The peelable release sheet may be configured to hold about 5 adhesive tapes. The peelable release sheet may be configured to hold about 4 adhesive tapes. The peelable release sheet may be configured to hold about 3 adhesive tapes. The peelable release sheet may be configured to hold about 2 adhesive tapes. The peelable release sheet may be configured to hold about 1 adhesive tape.

In some embodiments, the adhesive tape is applied to the skin and removed from the skin. After removing the used adhesive tape from the skin surface, the tape stripping method may further comprise storing the used tape on a placement area sheet, where the tape remains until the skin sample is isolated or otherwise utilized. The used tape is configured to be stored on the placement area sheet for at least 1 week at temperatures between −80° C. and 30° C. In some embodiments, the used tape is configured to be stored on the placement area sheet for at least 2 weeks, at least 3 weeks, at least 1 month, at least 2 months, at least 3 months, at least 4 months, at least 5 months, and at least 6 months at temperatures between −80° C. to 30° C.

In some embodiments, the placement area sheet comprises a removable liner, provided that prior to storing the used tape on the placement area sheet, the removable liner is removed. The placement area sheet may be configured to hold a plurality of adhesive tapes, including, but not limited to, 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The placement area sheet may be configured to hold about 12 adhesive tapes. The placement area sheet may be configured to hold about 11 adhesive tapes. The placement area sheet may be configured to hold about 10 adhesive tapes. The placement area sheet may be configured to hold about 9 adhesive tapes. The placement area sheet may be configured to hold about 8 adhesive tapes. The placement area sheet may be configured to hold about 7 adhesive tapes. The placement area sheet may be configured to hold about 6 adhesive tapes. The placement area sheet may be configured to hold about 5 adhesive tapes. The placement area sheet may be configured to hold about 4 adhesive tapes. The placement area sheet may be configured to hold about 3 adhesive tapes. The placement area sheet may be configured to hold about 2 adhesive tapes. The placement area sheet may be configured to hold about 1 adhesive tape.

In some embodiments, the used tape is stored so that the matrix containing, skin facing surface of the used tape is in contact with the placement area sheet. In some embodiments, the placement area sheet is a panel of the sample collection kit. In some embodiments, the sample collector may further comprise a clear panel. The sample collector may be labeled with a unique barcode that is assigned to a subject. In some embodiments, the sample collector comprises an area for labeling subject or patient information.

In some embodiments, the adhesive skin sample collection kit comprises a sample collector comprising adhesive tapes stored on a peelable release panel. In some embodiments, the sample collector further comprises a placement area panel with a removable liner. The tape stripping method may involve removing an adhesive tape from the sample collector peelable release panel, applying the adhesive tape to a skin sample, removing the used adhesive tape containing a skin sample and placing the used tape on the placement area sheet. In some embodiments, the placement area panel is a single placement area panel sheet. The identity of the skin sample collected may be indexed to the sample collector or placement area panel sheet by using a barcode or printing patient information on the collector or panel sheet. The indexed sample collector or placement sheet may be sent to a diagnostic lab for processing. The used tape may be configured to be stored on the placement panel for at least 1 week at temperatures between −80° C. and 25° C. In some embodiments, the used tape is configured to be stored on the placement area panel for at least 2 weeks, at least 3 weeks, at least 1 month, at least 2 months, at least 3 months, at least 4 months, at least 5 months, and at least 6 months at temperatures between −80° C. and 25° C. In some embodiments, the indexed sample collector or placement sheet is sent to a diagnostic lab using UPS or FedEx.

2. Collection of Samples Using Adhesive Patches

In some embodiments, the tape stripping method further comprises preparing the skin sample prior to application of the adhesive tape. Preparation of the skin sample may include, but is not limited to, removing hairs on the skin surface, cleansing the skin surface and/or drying the skin surface. In some embodiments, the skin surface is cleansed with an antiseptic including, but not limited to, alcohols, quaternary ammonium compounds, peroxides, chlorhexidine, halogenated phenol derivatives and quinolone derivatives. In some embodiments, the alcohol is about 0 to about 20%, about 20 to about 40%, about 40 to about 60%, about 60 to about 80%, or about 80 to about 100% isopropyl alcohol. In some embodiments, the antiseptic is 70% isopropyl alcohol.

In some embodiments, the tape stripping method is used to collect a skin sample from the surfaces including, but not limited to, the face, head, neck, arm, chest, abdomen, back, leg, hand or foot. In some embodiments, the skin surface is not located on a mucous membrane. In some embodiments, the skin surface is not ulcerated or bleeding. In some embodiments, the skin surface has not been previously biopsied. In some embodiments, the skin surface is not located on the soles of the feet or palms.

The tape stripping method, devices, and systems described herein may be useful for the collection of a skin sample from a skin lesion. A skin lesion may be a part of the skin that has an appearance or growth different from the surrounding skin. In some embodiments, the skin lesion is pigmented. A pigmented lesion may include, but is not limited to, a mole, dark colored skin spot and a melanin containing skin area. In some embodiments, the skin lesion is from about 5 mm to about 16 mm in diameter. In some embodiments, the skin lesion is from about 5 mm to about 15 mm, from about 5 mm to about 14 mm, from about 5 mm to about 13 mm, from about 5 mm to about 12 mm, from about 5 mm to about 11 mm, from about 5 mm to about 10 mm, from about 5 mm to about 9 mm, from about 5 mm to about 8 mm, from about 5 mm to about 7 mm, from about 5 mm to about 6 mm, from about 6 mm to about 15 mm, from about 7 mm to about 15 mm, from about 8 mm to about 15 mm, from about 9 mm to about 15 mm, from about 10 mm to about 15 mm, from about 11 mm to about 15 mm, from about 12 mm to about 15 mm, from about 13 mm to about 15 mm, from about 14 mm to about 15 mm, from about 6 to about 14 mm, from about 7 to about 13 mm, from about 8 to about 12 mm and from about 9 to about 11 mm in diameter. In some embodiments, the skin lesion is from about 10 mm to about 20 mm, from about 20 mm to about 30 mm, from about 30 mm to about 40 mm, from about 40 mm to about 50 mm, from about 50 mm to about 60 mm, from about 60 mm to about 70 mm, from about 70 mm to about 80 mm, from about 80 mm to about 90 mm, and from about 90 mm to about 100 mm in diameter. In some embodiments, the diameter is the longest diameter of the skin lesion. In some embodiments, the diameter is the smallest diameter of the skin lesion.

3. Adhesive Skin Sample Collection Kit

In some embodiments, an adhesive skin sample collection kit is used to obtain tissue samples from an area of skin affected by a skin condition. Embodiments of adhesive skin sample collection system and methods disclosed by US Patent Publication 2018/0110500, the entire disclosure of which is incorporate herein by reference.

The adhesive skin sample collection kit may comprise at least one adhesive tape, a sample collector, and instructions for use sheet. In some embodiments, the sample collector is a sample collector comprising a peelable release panel comprising at least one adhesive tape, a placement area panel comprising a removable liner, and a clear panel. The sample collector may further comprise a barcode and/or an area for transcribing patient information. The adhesive skin sample collection kit may be configured to include a plurality of adhesive tapes, including but not limited to 12, 11, 10, 9, 8, 7, 6, 5, 4, 3, 2, 1, from about 2 to about 8, from about 2 to about 7, from about 2 to about 6, from about 2 to about 4, from about 3 to about 6, from about 3 to about 8, from about 4 to about 10, from about 4 to about 8, from about 4 to about 6, from about 4 to about 5, from about 6 to about 10, from about 6 to about 8, or from about 4 to about 8. The instructions for use sheet may provide the kit operator all of the necessary information for carrying out the tape stripping method. The instructions for use sheet may include diagrams to illustrate the tape stripping method.

In some embodiments, the instructions are provided to perform one or more of the following: marking the patch to approximately a size of a lesion on a skin; peel the patch slowly; and peel at an angle greater than about perpendicular to the skin surface. In some embodiments, slowly is indicated as less than about 0.5, 0.7, 0.8. 0.9, 1, 1.1, 1.2, 1.5, 2.0, or 2.5 linear inches peeled per about five seconds. In some embodiments, slowly is indicated as less than about 0.5, 0.7, 0.8. 0.9, 1, 1.1, 1.2, 1.5, 2.0, or 2.5 linear inches peeled per about ten seconds. In some embodiments, slowly is indicated as less than about 0.5, 0.7, 0.8. 0.9, 1, 1.1, 1.2, 1.5, 2.0, or 2.5 linear inches peeled per about three seconds.

In some embodiments, the adhesive skin sample collection kit provides all the necessary components for performing the tape stripping method. In some embodiments, the adhesive skin sample collection kit includes a lab requisition form for providing patient information. In some embodiments, the kit further comprises accessory components. Accessory components may include, but are not limited to, a marker, a resealable plastic bag, gloves and a cleansing reagent. The cleansing reagent may include, but is not limited to, an antiseptic such as isopropyl alcohol. The components of the skin sample collection kit may be provided in a cardboard box. A kit described herein may comprise a means for preservation or storage of a collected skin sample. In some instances, a kit for non-invasive collection and analysis of a skin sample comprises at least one adhesive patch, wherein the least one adhesive patch comprises: a backing layer comprising a collection area; a non-adhesive handling area; an adhesive matrix on a surface of the collection area, wherein the adhesive matrix is configured to adhere to an amount of a skin sample; and a return pouch sized and shaped to receive the at least one adhesive patch, the return pouch comprising a preservative. In some instances, the preservative is a desiccant. In some instances, the preservative is configured to prevent degradation of biological molecules sampled using the collector kit. In some instances, the desiccant is configured to prevent the activity of nucleases in the skin sample. In some instances, the desiccant is configured to prevent degradation of nucleic acids in the sample. In some instances, the desiccant is configured to prevent the activity of RNases in the skin sample. In some instances, the amount of the desiccant is from about 0.5 grams to about 5 grams, about 0.1 grams to about 10 grams, about 0.1 grams to about 5 grams, about 0.5 grams to about 5 grams, about 0.1, 0.5, 1, 1.5, 2.0, 2.5, 3, 3.5, 4, 4.5, 5, 5.5, 6, 8, 10, 12, 15 or about 20 grams. In some instances, the kit comprises a return pouch. In some instances, the return pouch is plastic or foil. In some instances, the return pouch is sealable. In some instances, the desiccant is silica gel.

Skin collector kits may comprise one or more water soluble components. In some embodiments, the adhesive patch is water soluble. In some embodiments, one or more of the backing layer and adhesive matrix are water soluble. In some embodiments, the placement area sheet is water soluble. In some embodiments, backing layer or adhesive matrix is configured to dissolve during skin sample lysis. In some embodiments, the adhesive matrix comprises at least 3, 5, 8, 10, 11, 12, 13, 14, 15, 18, 20, or at least 25 oz/in² loop tackiness, including increments therein. In some embodiments, the adhesive matrix comprises 3-25, 3-20, 3-15, 5-20, 8-20, 10-15, 15-24, 10-20, or 1-20 oz/in² loop tackiness. In some embodiments, the adhesive matrix comprises a working temperature range from −40 to 176° F., −40 to 150° F., −40 to 130° F., −30 to 176° F., −20 to 176° F., −10 to 176° F., or −40 to 200° F. In some embodiments, the backing layer comprises at least 5, 8, 10, 12, 15, 18, 20, 23, 25, 30, or at least 55 lb/inch tensile force, including increments therein. In some embodiments, the backing layer comprises about 5, 8, 10, 12, 15, 18, 20, 23, 25, 30, or about 55 lb/inch tensile force, including increments therein. In some embodiments, the backing layer comprises 5-55, 5-40, 5-30, 5-25, 1-50, 10-20, 10-30, 15-30, 15-45, 20-45, 25-40, 30-50, or 25-60 lb/inch tensile force. In some embodiments, the backing layer comprises about 50, 80, 100, 120, 150, 180, 200, 230, 250, 300, 400, or about 500 mN tear strength, including increments therein. In some embodiments, the backing layer comprises 50-550, 50-400, 50-300, 50-250, 100-500, 100-200, 100-300, 150-300, 150-450, 200-450, 250-400, 300-500, or 250-600 mN tear strength. In some instances, one or more components of the skin collector kit may be water soluble. In some instances, the adhesive patch is water soluble. In some instances, one or more of the backing layer and adhesive matrix are water soluble. In some instances, the placement area sheet is water soluble. In some instances, backing layer or adhesive matrix is configured to dissolve during skin sample lysis. In some embodiments, the adhesive patch is dissolvable in no more than 10, 15, 20, 30, 40, 50, 60, 90, or not more than 120 seconds, including increments therein. In some embodiments, the adhesive patch is dissolvable in no more than 10, 15, 20, 30, 40, 50, 60, 90, or not more than 120 seconds in an aqueous solution. In some embodiments, the adhesive patch is dissolvable in no more than 10, 15, 20, 30, 40, 50, 60, 90, or not more than 120 seconds in an aqueous solution having a temperature of no more than 30 degrees C. In some embodiments, the adhesive patch is dissolvable in no more than 10, 15, 20, 30, 40, 50, 60, 90, or not more than 120 seconds in an aqueous solution having a temperature of no more than 20 degrees C. In some embodiments, wherein the adhesive patch has shelf life of at least 1, 2, 3, 6, 8, 12, 14, 16, or at least 24 months, including increments therein. In some embodiments, wherein the adhesive patch has a shelf life of at least 1, 2, 3, 6, 8, 12, 14, 16, or at least 24 months, including increments therein, at a temperature of no more than 30 degrees C.

In some embodiments, water soluble adhesives are formed by the copolymerization of a hydrophilic monomer with a monomer that is used in a more conventional adhesive resin. The formulations of such resins may be performed with various type of water soluble/dispersible salts, plasticizers, tackifiers and surfactants. For this reason, polymers like polyvinylpyrrolidone, polyacrylamide, polyacrylic acid, polyvinyl ethers, cellulose ethers, natural or synthetic gums and polyethers (e.g., polyethylene glycol) maybe used to formulate adhesives. A variety of surfactants may be utilized as tackifiers and plasticizers (such as ethoxylates, glucosides, rosins, and polyols) to improve the adhesion.

In some embodiments, a conventional acrylic adhesive is converted to a water soluble adhesive is the neutralization of carboxylic group in the pendant group. In some embodiments, the resultant polymer is optionally plasticized with polyethylene glycol or polypropylene glycol. In some embodiments, a solution polymerization of typical (adhesive) monomers, such as butyl acrylate, acrylic acid, di-2-ethylhexyl fumarate and vinyl acetate are copolymerized. In some embodiments, copolymerization is followed by the addition of an ethoxylated tert-N-alkyl diamine (an ethoxylated surfactant) as a plasticizer/tackifier, and potassium hydroxide (neutralization agent).

In some embodiments, a water soluble adhesive, based on acrylic acid and acrylamide, a polyhydric alcohol surfactant (tackifier/plasticizer), and a caustic (neutralization agent) was described is used as an adhesive. In some embodiments, copolymers of acrylic acid and acrylates are neutralized with aminopropanol followed by the addition of glycol ether to produce a water soluble adhesive. In some embodiments, copolymers of 2-ethylhexyl acrylate, hydroxyethyl methacrylate, and acrylic acid are neutralized with sodium hydroxide in methanol to make water soluble adhesive using polyethylene glycol (tackifier/plasticizer) and polypropylene glycol diglycidyl ether (tackifier/plasticizer). Polyethylene glycol, polypropylene glycol or similar hydrophilic polymers or surfactants with hydroxyl or amine groups may be grafted to acrylic acid pendant groups on the adhesive polymers to make them water soluble. In some embodiments, a backing sheet comprised of polyvinyl alcohol, cellulose ethers, and blends of such polymers with water dispersible/soluble additives and other thermoplastics are utilized.

B. Tissue Sampling and Cellular Material

The methods, devices, and systems provided herein may involve applying an adhesive or other similar tape to the skin in a manner so that an effective or sufficient amount of a tissue, such as a skin sample, adheres to the adhesive matrix of the adhesive tape. In some embodiments, the effective or sufficient amount of a skin sample is an amount that removably adheres to a material, such as the matrix or adhesive tape. The adhered skin sample, in some embodiments, comprises cellular material including nucleic acids and proteins. In some embodiments, the nucleic acid is RNA or DNA. An effective amount of a skin sample may contain an amount of cellular material sufficient for performing a diagnostic assay. In some embodiments, the diagnostic assay is performed using the cellular material isolated from the adhered skin sample on the used adhesive tape. In some embodiments, the diagnostic assay is performed on the cellular material adhered to the used adhesive tape. In some embodiments, an effect amount of a skin sample comprises an amount of RNA sufficient to perform a gene expression analysis. Sufficient amounts of RNA include picogram, nanogram, and microgram quantities.

In some embodiments, the adhered skin sample comprises cellular material including nucleic acids such as RNA or DNA, or a polypeptide such as a protein, in an amount that is at least about 1 picogram. In some embodiments, the amount of cellular material is no more than about 1 nanogram. In some embodiments, the amount of cellular material is no more than about 1 microgram. In still some embodiments, the amount of cellular material is no more than about 1 gram.

In some embodiments, the amount of cellular material is from about 1 picogram to about 1 gram. In some embodiments, the cellular material comprises an amount that is from about 50 microgram to about 1 gram, from about 100 picograms to about 500 micrograms, from about 500 picograms to about 100 micrograms, from about 750 picograms to about 1 microgram, from about 1 nanogram to about 750 nanograms, or from about 1 nanogram to about 500 nanograms.

In some embodiments, the amount of cellular material, including nucleic acids such as RNA or DNA, or a polypeptide such as a protein, comprises an amount that is from about 50 microgram to about 500 microgram, from about 100 microgram to about 450 microgram, from about 100 microgram to about 350 microgram, from about 100 microgram to about 300 microgram, from about 120 microgram to about 250 microgram, from about 150 microgram to about 200 microgram, from about 500 nanograms to about 5 nanograms, or from about 400 nanograms to about 10 nanograms, or from about 200 nanograms to about 15 nanograms, or from about 100 nanograms to about 20 nanograms, or from about 50 nanograms to about 10 nanograms, or from about 50 nanograms to about 25 nanograms.

In some embodiments, the amount of cellular material, including nucleic acids such as RNA or DNA, or a polypeptide such as a protein, is less than about 1 gram, is less than about 500 micrograms, is less than about 490 micrograms, is less than about 480 micrograms, is less than about 470 micrograms, is less than about 460 micrograms, is less than about 450 micrograms, is less than about 440 micrograms, is less than about 430 micrograms, is less than about 420 micrograms, is less than about 410 micrograms, is less than about 400 micrograms, is less than about 390 micrograms, is less than about 380 micrograms, is less than about 370 micrograms, is less than about 360 micrograms, is less than about 350 micrograms, is less than about 340 micrograms, is less than about 330 micrograms, is less than about 320 micrograms, is less than about 310 micrograms, is less than about 300 micrograms, is less than about 290 micrograms, is less than about 280 micrograms, is less than about 270 micrograms, is less than about 260 micrograms, is less than about 250 micrograms, is less than about 240 micrograms, is less than about 230 micrograms, is less than about 220 micrograms, is less than about 210 micrograms, is less than about 200 micrograms, is less than about 190 micrograms, is less than about 180 micrograms, is less than about 170 micrograms, is less than about 160 micrograms, is less than about 150 micrograms, is less than about 140 micrograms, is less than about 130 micrograms, is less than about 120 micrograms, is less than about 110 micrograms, is less than about 100 micrograms, is less than about 90 micrograms, is less than about 80 micrograms, is less than about 70 micrograms, is less than about 60 micrograms, is less than about 50 micrograms, is less than about 20 micrograms, is less than about 10 micrograms, is less than about 5 micrograms, is less than about 1 microgram, is less than about 750 nanograms, is less than about 500 nanograms, is less than about 250 nanograms, is less than about 150 nanograms, is less than about 100 nanograms, is less than about 50 nanograms, is less than about 25 nanograms, is less than about 15 nanograms, is less than about 1 nanogram, is less than about 750 picograms, is less than about 500 picograms, is less than about 250 picograms, is less than about 100 picograms, is less than about 50 picograms, is less than about 25 picograms, is less than about 15 picograms, or is less than about 1 picogram, including increments therein.

### II. PLATFORM FOR SCANNING OF SAMPLE COLLECTORS

In some embodiment, the method further comprises placing a plurality of the tissue sample collectors on a platform, whereby an array of sample collectors is formed. In some embodiments, the platform is a sheet. In some embodiments, the sheet is comprised of plastic, a polymer, or other suitable material to provide a substrate for an array of sample collectors. In some embodiments, a protective cover is applied to the array to protect the cell samples during scanning and cutting. In some embodiment, the protective cover may comprise transparent or translucent sheeting. In some embodiments, as described herein, sample strips with adhesive skin sample collection patches are loaded onto a matrix sheet for proper orientation and placement. In some embodiments, the matrix sheet comprises one or more though holes which correspond to alignment pins provided on a cutter and/or scanner stage to facilitate proper placement and orientation.

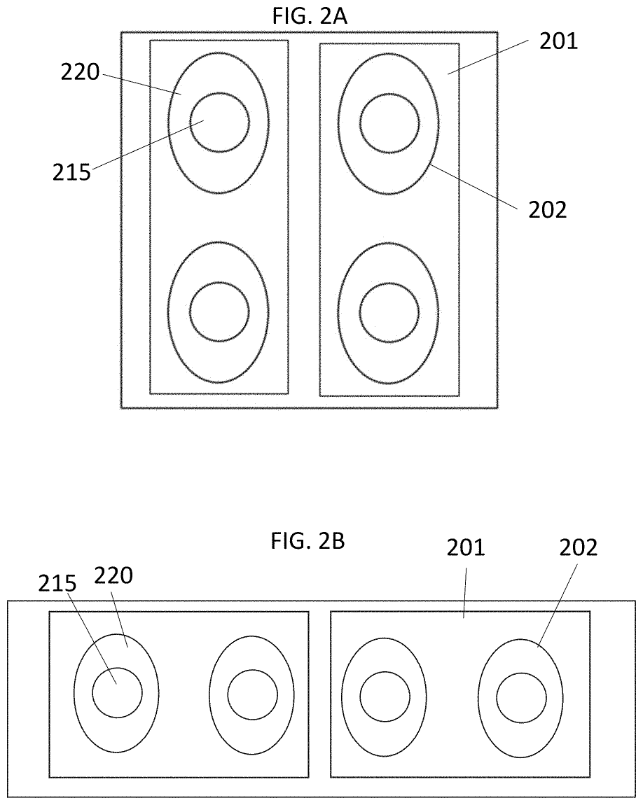

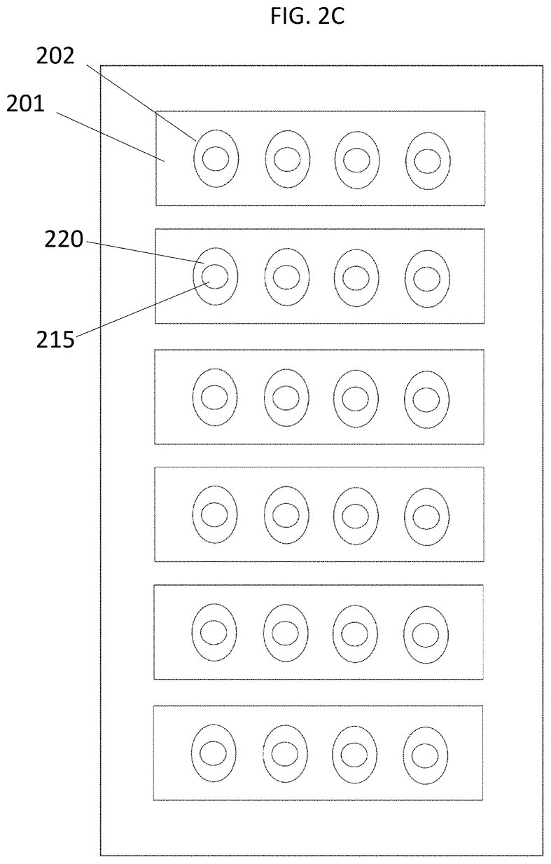

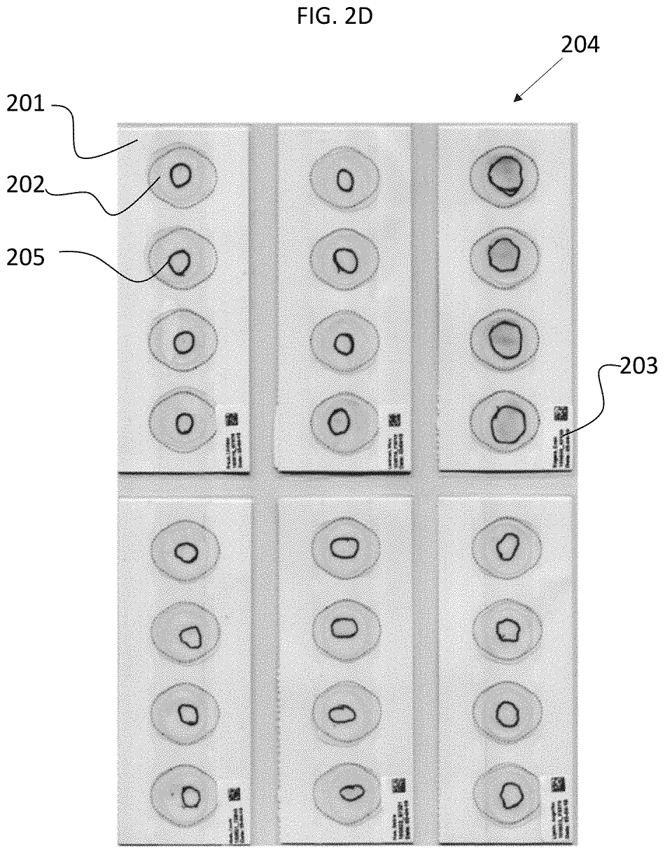

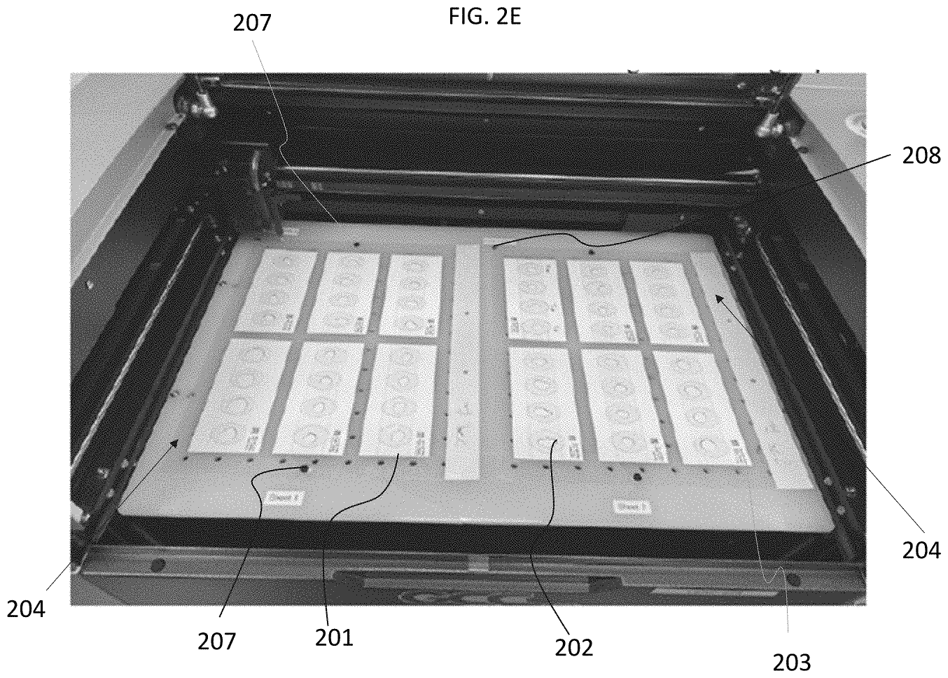

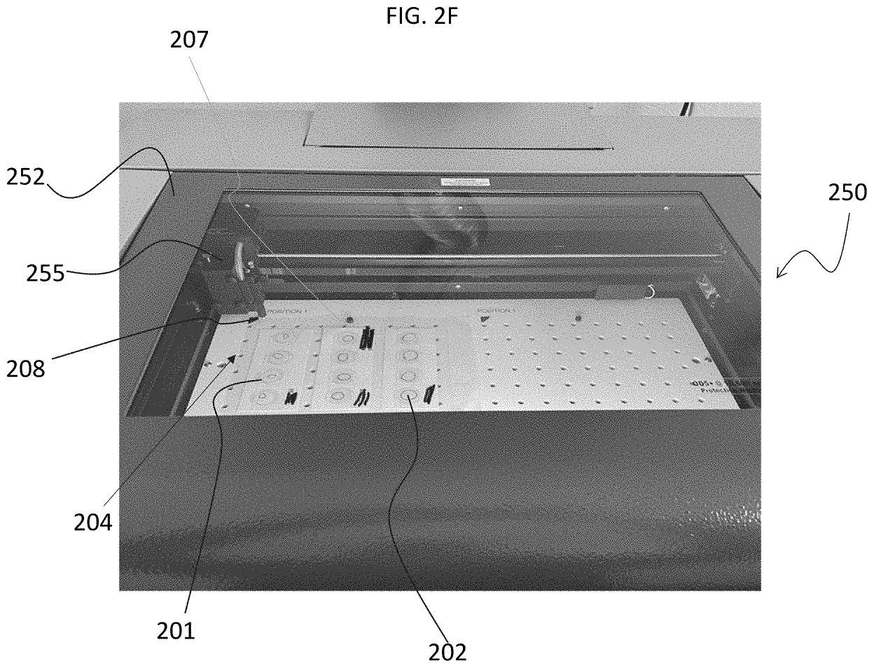

With reference to FIGS. 2A-2D, embodiments of sample collector arrays are depicted. With reference to FIG. 2A, an array may be formed by two sample panels or strips 201, wherein each sample collector comprises two sample collectors 202, forming a 2 by 2 array of tissue samples. The samples 202 may be provided by adhesive patches. In some embodiments, the adhesive patches comprise a collection area 215 and a handling area 220, which are further described herein. With reference to FIG. 2C, an array may be formed by six sample strips 201, wherein each sample strip comprises four sample collectors 202, forming a 4 by 6 array of tissue sample collectors.

With reference to FIG. 2B, a plurality of sample strips 201 of sample collectors 202 may be longitudinally connected for form a linear array. The arrangement may be similar to a film strip. In some embodiments, a linear array comprises any number of sample collectors, and may be provided as with any length. In some embodiments, a linear array allows for continuous processing and cutting in a serial procedure. In some embodiments, a linear array may be fed into a system, wherein a sample 202 is scanned by one or more image sensors which identify a delineation between the cells of interest and the surrounding portion of the sample collector. The linear array may then be positioned, such that the sample collector 202 which has been scanned is cut along the boundary formed by the delineation. At the same time, a succeeding sample of the linear array may be scanned. This process may be repeated along the entire length of the array until all samples of the array are cut.

With reference to FIG. 2D, a plurality of sample collectors 202 may be arranged to form a sheet. In some embodiments, a sheet of sample collectors comprises a set of 6 sample strips 201 arranged in 2 rows and 3 columns or 3 rows and 2 columns, depending on the orientation. In some embodiments, each sample strip 201 comprises 4 sample collectors 202 arranged in a linear fashion. In some embodiments, a sheet comprises 32 sample collectors to be processed by the system and methods herein.

In some embodiments, each of the samples comprise a demarcation 205. In some embodiments, the demarcation 205 is provided by a patient or person assisting the patient during sample collection. The demarcation 205 may be provided by a marker. The marker may be part of a kit supplied to the patient along with the sample collectors 202 and sample strips 201. In some embodiments, each sample strip 201 is provided with an identification tag 203. In some embodiments, the identification tags 203 comprise a scannable code containing sample and/or patient information. In some embodiments, the identification tag 203 is scanned to correspond the samples to a patient, from which the tissue samples were taken. In some embodiments, the identification tag comprises a machine readable code such as a barcode, QR code, or the like.

In some embodiments, the systems and methods herein allow for processing of about 16 samples to about 2,096 samples per given unit time, such as per minute. In some embodiments, the systems and methods herein allow for processing of about 16 samples to about 32 samples, about 16 samples to about 64 samples, about 16 samples to about 128 samples, about 16 samples to about 256 samples, about 16 samples to about 512 samples, about 16 samples to about 1,048 samples, about 16 samples to about 2,096 samples, about 32 samples to about 64 samples, about 32 samples to about 128 samples, about 32 samples to about 256 samples, about 32 samples to about 512 samples, about 32 samples to about 1,048 samples, about 32 samples to about 2,096 samples, about 64 samples to about 128 samples, about 64 samples to about 256 samples, about 64 samples to about 512 samples, about 64 samples to about 1,048 samples, about 64 samples to about 2,096 samples, about 128 samples to about 256 samples, about 128 samples to about 512 samples, about 128 samples to about 1,048 samples, about 128 samples to about 2,096 samples, about 256 samples to about 512 samples, about 256 samples to about 1,048 samples, about 256 samples to about 2,096 samples, about 512 samples to about 1,048 samples, about 512 samples to about 2,096 samples, or about 1,048 samples to about 2,096 samples. In some embodiments, the systems and methods herein allow for processing of about 16 samples, about 32 samples, about 64 samples, about 128 samples, about 256 samples, about 512 samples, about 1,048 samples, or about 2,096 samples, including increments therein. In some embodiments, the systems and methods herein allow for processing of at least about 16 samples, about 32 samples, about 64 samples, about 128 samples, about 256 samples, about 512 samples, or about 1,048 samples, including increments therein.

Formation of arrays from the samples and sample collectors should not be limited by the embodiments depicted herein. Various sample and array configurations may be formed and provided to be cut by the system described herein.

A. Scanning of Sample Collectors

In some embodiments herein, a method of scanning one or more samples or an array of sample collectors to identify a delineation of cells of interest within each tissue sample collector is provided herein.

In some embodiments, the scanning comprises identifying a delineation of first and second areas, for example the cells of interest obtained from a lesion area from other cells obtained from a non-lesion surrounding area for each of the one or more samples. In some embodiments, the identifying includes receiving data obtained during a non-invasive adhesive sampling process. In some embodiments, data may be received from a digitized image. In some embodiments, images are collected by a software application feature used by patient or clinician during sample collection process and used to identify a delineation of cells of interest obtained from a lesion area from other cells obtained from a non-lesion surrounding area for each of the one or more samples.

With reference to FIG. 2E, one or more sheets 204 of sample strips 201 comprising multiple sample collectors 202 are provided in a system for scanning and/or cutting the sample collectors at a delineation representative of a border of one or more lesions, moles, or regions of interest, from which a tissue sample is collected, according to some embodiments. In some embodiments, a combination scanner/cutter apparatus is utilized. In some embodiments, the system comprises a separate scanner and cutter. In some embodiments, as depicted in FIG. 2E, the apparatus for scanning and/or cutting allows for two sheets 204 to be processed at time. In some embodiments, the apparatus for scanning and/or cutting processes at least 1 sheet 204 of sample collectors at a time, 2 sheets 204 of sample collectors at a time, 4 sheets 204 of sample collectors at a time, 6 sheets 204 of sample collectors at a time, 10 sheets 204 of sample collectors at a time, 20 sheets 204 of sample collectors at a time, 50 sheets 204 of sample collectors at a time, or 100 sheets 204 of sample collectors at a time, including increments therein.

In some embodiments, the platform for scanning and/or cutting comprises one or more alignment posts 207 for aligning the sheets 204. In some embodiments, the sheets 204 comprise corresponding through holes to receive the alignment posts 207. In some embodiments, wherein the system comprises separate scanning and cutting apparatuses, both the scanner and the cutter comprise corresponding alignment posts are utilized to maintain a proper position of the sheets of sample collectors when they are transferred from the scanner to the cutter.

In some embodiments, both sides of the sheets 204 are scanned. In some embodiments, the back side of each sample strip 201 (the surface of the strip opposite of where the adhesive sample collectors are placed) comprises further identification information such as patient information, serial numbers of each sample strip, time of collection, etc. In some embodiments, the scanner of the system scans both sides of the sheet of sample strips simultaneously. In some embodiments, the sheet of sample strips must be flipped for both sides of the strips to be scanned. In some embodiments, flipping of the sheets is automated. In some embodiments, flipping of the sheets is done by a technician.

In some embodiments, each sheet comprises an alignment corner 208 which is indented or otherwise marked to facilitate proper orientation of the sheet within the cutting and/or scanning apparatus. In some embodiments, markings on a surface of the scanner and/or cutting apparatus are provided for alignment with the alignment corner of the sheet of samples. In some embodiments, wherein the system comprises separate scanning and cutting apparatuses, both the scanner and the cutter comprise corresponding markings for aligning the marked or indented corner of the sheet to maintain a proper position of the sheets of sample collectors when they are transferred from the scanner to the cutter.

FIG. 2F depicts sample strips a sheet 204 of sample strips 201 comprising multiple sample collectors 202 are provided in a cutting apparatus 250 comprising a cutting head 255 for cutting the sample collectors at a delineation representative of a border of one or more lesions, moles, or regions of interest, from which a tissue sample is collected, according to some embodiments. In some embodiments, the cutting apparatus 250 comprises a lid or cover 252 to prevent contamination of the samples or impedance of the cutting process by external forces, objects falling into the workspace, etc. In some embodiments, the cutting device comprises one or more alignment posts 207 for aligning the sheets 204. In some embodiments, the sheets 204 comprise corresponding through holes to receive the alignment posts 207. In some embodiments, the scanner and the cutter comprise corresponding alignment posts are utilized to maintain a proper position of the sheets of sample collectors when they are transferred from the scanner to the cutter. In some embodiments, each sheet comprises an alignment corner 208 which is indented or otherwise marked to facilitate proper orientation of the sheet within the cutting apparatus.

In some embodiments, the skin sample comprises a lesion, and wherein the lesion is suspected to be melanoma, lupus, rubeola, acne, hemangioma, psoriasis, eczema, candidiasis, impetigo, shingles, leprosy, Crohn's disease, inflammatory dermatoses, bullous diseases, infections, basal cell carcinoma, actinic keratosis, seborrheic keratosis, Merkel cell carcinoma, sebaceous carcinoma, squamous cell carcinoma, or dermatofibrosarcoma protuberans.

In some embodiments, the scanning is performed by a computerized scanning the array of collected samples to identify markings on each of the one or more samples. The scanner may comprise a laser scanner, photo scanner, or the like. In some embodiments, the markings include a demarcation that was applied during the non-adhesive sampling process. In some embodiments, the demarcation includes a pen or other mark creating by a marking utensil. In some embodiments, the demarcation is made by a user applying the sample collector to an area affected by a skin condition. In some embodiments, the user marks the sample collector on a surface opposite of the adhesive matrix applied to the skin. In some embodiments, an adhesive of a sample collection kit patch includes pre-printed demarcations used by the patient to align the lesion/area of interest. The pre-printed demarcations may be dependent upon (specific to) indication/disease identification.

1. Scanning Software Applications

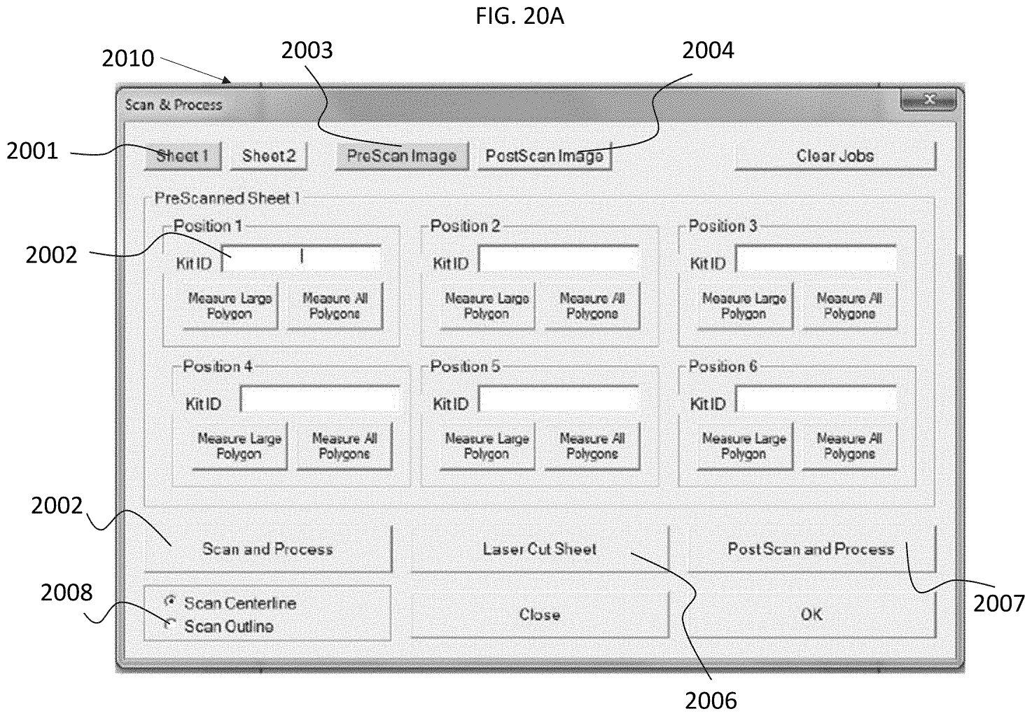

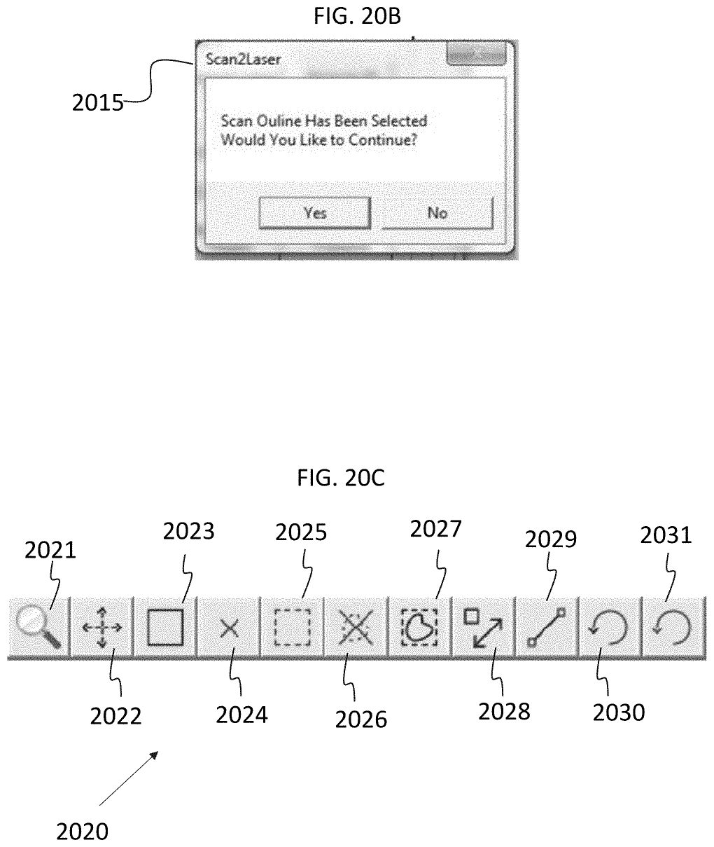

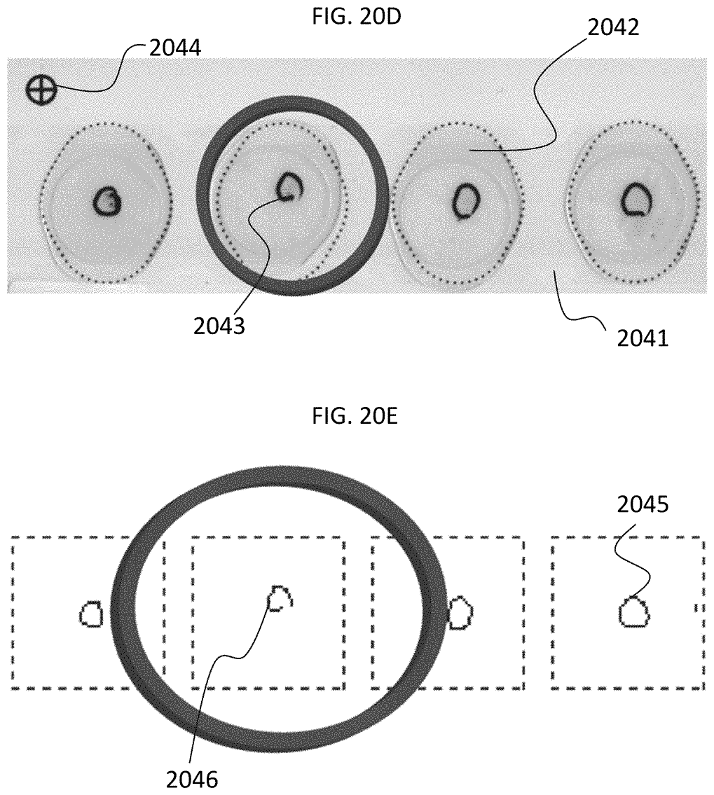

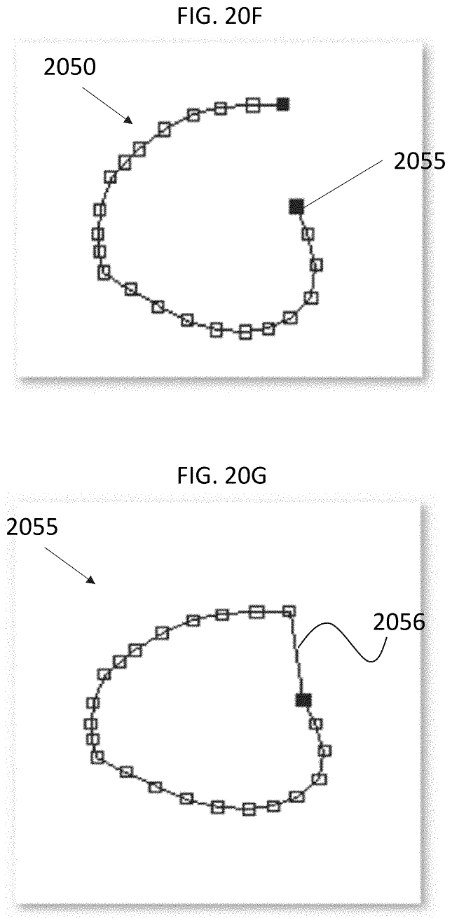

With reference to FIGS. 20A-20G, a software application executed by a computing device of the system may comprise custom graphical user interfaces (GUIs) for initiating scans and verifying and/or editing delineations at which the sample collector is to be cut.

With reference to FIG. 20A, an exemplary GUI of a scan initiation user interface 2010 is depicted, according to some embodiments. In some embodiments, the scan initiation user interface comprises one or more sheet selection buttons 2001. In some embodiments, wherein multiple sheets are being scanned, the sheet selection button allows each sheet to be selected for verification and/or input of information. In some embodiments, the scan initiation GUI 2010 comprises one or more identification fields 2002 for receiving information corresponding to each sample strip (e.g., sample strips 201 as depicted in FIG. 2E). In some embodiments, the identification fields are automatically populated. In some embodiments, automatic population is facilitated by scanned images of identification information provided on a back side of the sample strips or on an identification tag provided on each sample strip (i.e., identification tag 203 depicted in FIG. 2E).

In some embodiments, each identification field corresponds to a sample strip. In some embodiments, the scan initiation GUI 2010 comprises an identification field 2002 for entering a “Kit ID” number for each sample strip. In some embodiments, each sample strip

In some embodiments, the scan initiation GUI 2010 comprises a selectable “PreScan Image” button 2003 for viewing and/or saving images of the sheets prior to being cut. In some embodiment, the preview or prescan image comprises a quick scan image of the sheets taken at a lower resolution. In some embodiments, the scan initiation GUI 2010 comprises a selectable “PostScan Image” button 2004 for viewing and/or saving images of the sheets after they have been scanned and cut.

In some embodiments, the scan initiation GUI 2010 comprises a selectable “Scan and Process” button 2005. In some embodiments, the scan and process button initiates scanning of the samples and identification of delineations representative of a border of the lesion to which the sample collectors were applied to. In some embodiments, identification of delineation comprises identification of demarcations provided on the sample collectors, as described herein.

In some embodiments, the scan initiation GUI 2010 comprises a selectable “Laser Cut Sheet” button 2006. In some embodiments, the “Laser Cut Sheet” button initiates cutting of the samples and identified and/or edited of delineation representative of a border of the lesion to which the sample collectors were applied to.

In some embodiments, the scan initiation GUI 2010 comprises a selectable “PostScan and Process” button 2007. In some embodiments, the “PostScan and Process” button initiates scanning of the matrix sheets and cut adhesive patches after the cut portions containing in the samples have been removed.

In some embodiments, a mode selection box 2008 provides two radio buttons to select between a “Scan Centerline” and “Scan Outline” button. In some embodiments, the “Scan Centerline” button is utilized when a demarcation provided on the adhesive patches is an outline of the border. In some embodiments, the “Scan Outline” button is utilized when a demarcation provided on the adhesive patches is a filled in shape covering the entire region representative of the lesion or mole of which the tissue sample was obtained. In some embodiments, wherein images captured from a patient facing software application are utilized to identify a delineation, the “Scan Outline” button should be selected. In some embodiments, as depicted in FIG. 20B, once the “Scan and Process” button is selected, a window 2015 is displayed to confirm the selection made using the mode selectable box 2008.

With reference to FIG. 20C, an exemplary tool bar 2020 for verifying and/or editing delineations is shown. In some embodiments, a zoom tool 2021 is used to enlarge the screen images. In some embodiments, the zoom took is used by dragging the cursor diagonally over an image to create a box that will become the border for the image enlargement. In some embodiments, left clicking the cursor on the image with the zoom tool activated will enlarge the image by a predetermined amount (e.g., 10%). In some embodiments, right clicking the cursor on the image with the zoom tool activated will reduce the image by a predetermined amount (e.g., 10%). In some embodiments, a pan tool 2022 is used to shift the location of the page within the screen view. In some embodiments, a zoom out tool 2023 is selectable to zoom out to view the entire image page. In some embodiments, a point delete tool 2024 is provided. In some embodiments, the point delete tool is used to eliminate unnecessary points from a delineation overlay, which may be caused by debris or other abnormalities. This function may be used with an enlarged view of polygon shapes, wherein the cursor is placed on a single point and a left click is used to delete point of the polygon. In some embodiments, a delete selected points tool 2025 is provided. In some embodiments, the delete selected point tool is utilized by dragging a box around all points of a delineation which are desired to be deleted. In some embodiments, a polygon delete tool 2026 is provided. In some embodiments, the polygon delete tool 2026 is used for selecting to delete a single point of a polygon delineation to delete the entire delineation. In some embodiments, a crop tool 2027 is provided. In some embodiments, the crop tool is utilized by dragging a box around all points which are desired to be save, deleting all points outside of the box. In some embodiments, a move point tool 2028 is selected to enable selection and movement of a single point on a polygon delineation. In some embodiments, a connect polygon tool 2029 is provided. In some embodiments, the connect polygon tool is used to connect two endpoints of a polygon delineation to close the polygon. In some embodiments, an undo tool 2030 is provided to revert one step of editing. In some embodiments, an undo all tool 2031 is provided to revert to the original processed vectors that were applied prior to any editing.

With referenced to FIG. 20D, a scanned image 2040 of a sample strip 2041 with adhesive patches 2042 having demarcations is depicted, according to some embodiments. With reference to FIG. 20D, an incomplete demarcation 2043 is highlighted. In some embodiments, the sample strip further comprises a fiducial 2044 to ensure proper orientation and placement of the sample strip 2041. FIG. 20E depicts delineations 2045 generated from the demarcations shown in provided on the sample strip (2041 of FIG. 20D). In some embodiments, the delineations are representative of the line on which the sample will be cut. In some embodiments, the delineations are generated by using computer vision techniques, as disclosed herein. In some embodiments, an incomplete demarcation (2043 as depicted in FIG. 20D) will produce an incomplete delineation 2046.

FIGS. 20F and 20G depict a process for editing and closing an incomplete delineation polygon 2050. In some embodiments, each delineation polygon comprises multiple points 2051, which are able to be manipulated. In some embodiments, solid point boxes are end points of any individual polygon, and empty point boxes are the continuation of any individual polygon. With reference to FIG. 20F, an incomplete polygon 2050 is depicted, according to some embodiments, an incomplete polygon may occur when a demarcation is incomplete or when an anomaly has occurred. FIG. 20G depicts a completed or closed polygon delineation 2055, according to some embodiments. In some embodiments, to close the incomplete delineation polygon 2050, a connect polygon tool (2029 of FIG. 20D) is selected and the polygon is closed by selecting one endpoint of the polygon and connecting it to the other endpoint, which forms a new line 2056 to close the polygon delineation.

In some embodiments, computer vision techniques and methods are utilized to assess captured images of lesions or other skin conditions of interest. In some embodiments, computer vision techniques and methods are utilized to identify features of captured images of lesions or other skin conditions of interest. In some embodiments, computer vision techniques are utilized during computerized scanning to identify demarcations on an adhesive substrate prior to cutting of the substrate, as disclosed herein. In some embodiments, computer vision techniques are utilized to identify a border of a lesion during the image capturing process. The identified border may then be transmitted to the cutting system such that the cutting system may separate or cut the adhesive substrate at the identified border.

In some embodiments, computer vision techniques and methods are utilized to identify a size, shape, symmetry, elevation, variability, color, and/or border of a lesion or mole. In some embodiments, computer vision techniques may identify diffusion of a lesion border from captured images of the lesion. Two or more images of lesions may be analyzed and compared using computer vision techniques to highlight moles or pigmentation which may be concerning due to characteristics which do not match other moles or pigmentation on an individual. In some embodiments, computer vision methods are utilized to compare images of a skin condition captured over a duration of time. Computer vision methods may be utilized to compare changes in, for example, size, shape, symmetry, elevation, variability, and/or color of a lesion or mole. Computer vision techniques may be able to produce more accurate analysis of changes in a lesion over time than achievable by a trained physician comparing images which may have been captured under different lighting.

In some embodiments, feature detection and extraction methods are utilized to identify a region of interest, such as a region of a skin condition or lesion. In some embodiment, feature detection and extraction methods comprise computing processing of images to analyze contrasts in pixel brightness to recognize features. Feature detection and extractions methods may include edge detection, corner detection, blob detection, ridge detection, and combinations thereof.

In some embodiments, an edge detection algorithm is utilized to identify an outline or border of a lesion or skin condition. In some embodiments, a nearest neighbor, thresholding, clustering, partial differential equation, and/or other digital image processing methods are utilized to identify an outline or border of a lesion or skin condition. In some embodiments, such diagnoses are performed by identifying and categorizing sections or pixels and then labeling or annotating each section/pixel as “normal skin” or “lesion skin.” Canny, Deriche, differential, Sobel, Prewitt, and Roberts cross edge detection techniques may be utilized to identify a region or border of a skin condition or lesion. In some embodiments, Gaussian or Laplacian techniques are utilized to smooth or improve the accuracy of the identified region or border of a skin condition or lesion. Edge detection may also be utilized to evaluate diffusion in the border of a lesion.

In some embodiments, segmentation of symmetry regions from normal skin, segmentation of asymmetry regions from normal skin, segmentation of pigment network regions from normal skin, segmentation of blotches from normal skin, segmentation of dots/globules from normal skin, and segmentation of dermatoscopic features such as: pigment network, amorphous structureless areas (blotches), leaf-like areas, blue ovoid masses, milia-like cysts, fissures and comedo-like openings, blood vessels, etc. are achievable using feature detection and extraction techniques.

In some embodiments, the delineation is based on disease indication, disease progression state, type of lesion, size of lesion, or the like. In some embodiments, the delineation may be based on a disease indication from Skin Cancer Dx Assays, including detecting Melanoma (PLA) and Melanoma (Nevome), BCC and SCC (Carcinome); Skin Cancer Risk Assessments, including assessing UV damage; skin inflammatory diagnoses, including Psoriasis/Psoriatic, Arthritis, Atopic Dermatitis/Atopic Asthma, Lupus, Cutaneous T Cell Lymphoma (CTCL), Alopecia Areata, Drug Reactions; and Skin Health Assessments, including skin condition and age.

In some embodiments, resolution of delineation may be less critical, and the scanner simply delineates a border spaced a predetermined distance from a handling area of an adhesive patch. For example, this method may be applied for atopic dermatitis and other inflammatory conditions.

In some embodiments, delineation is based on the intended testing/analysis to be performed on the tissue samples. Information regarding the analysis to be performed may be used to determine how to delineate. In some embodiments, the type of disease identification, type of dermatological lesion, suspected disease state, and specified one or analyses may be used to determine the delineation.

In some embodiments, the delineation includes first and second delineations. In some embodiments, both the lesion area and non-lesion areas are of interest and the borders each of the areas of interest are delineated, as described herein. In some embodiments, the delineation includes first and second delineations, where two smaller lesions are identified during the evaluation, as further described herein.

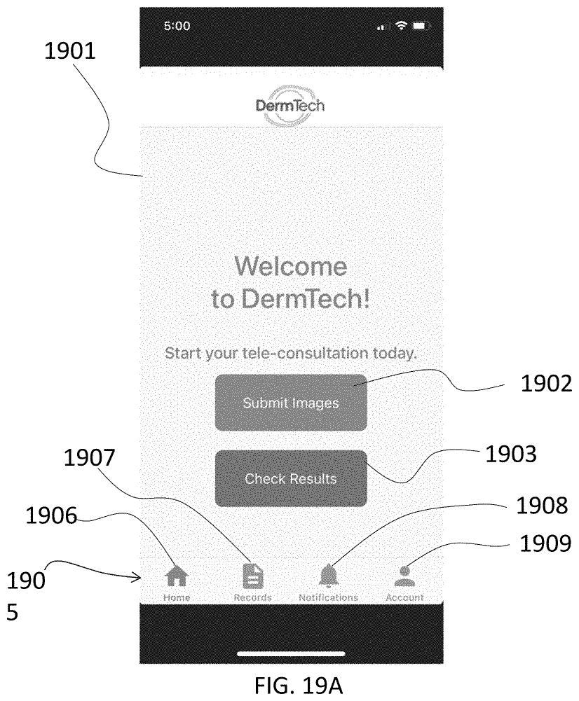

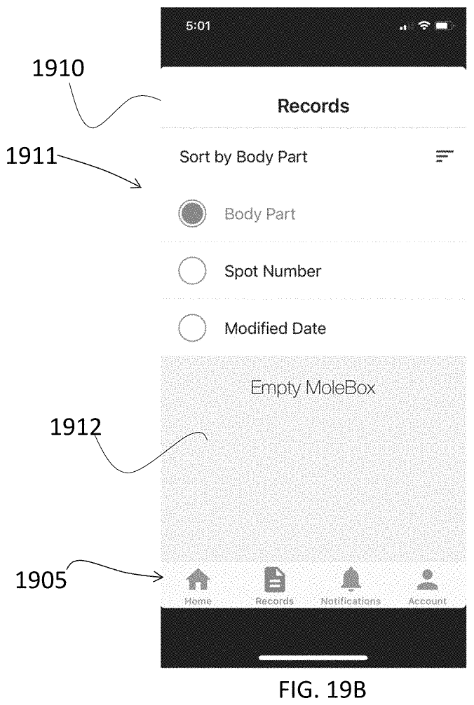

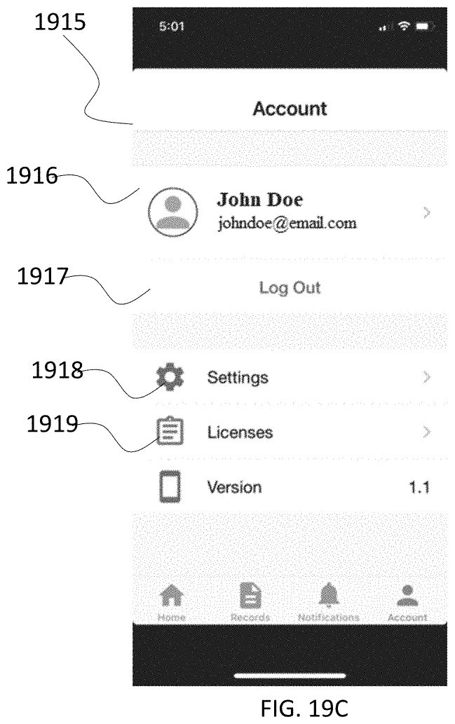

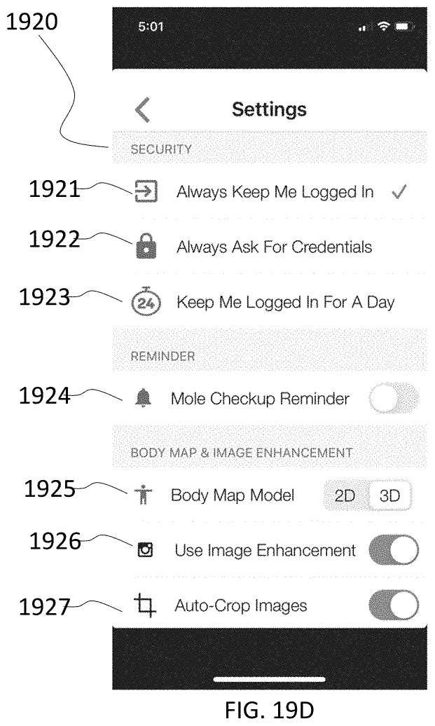

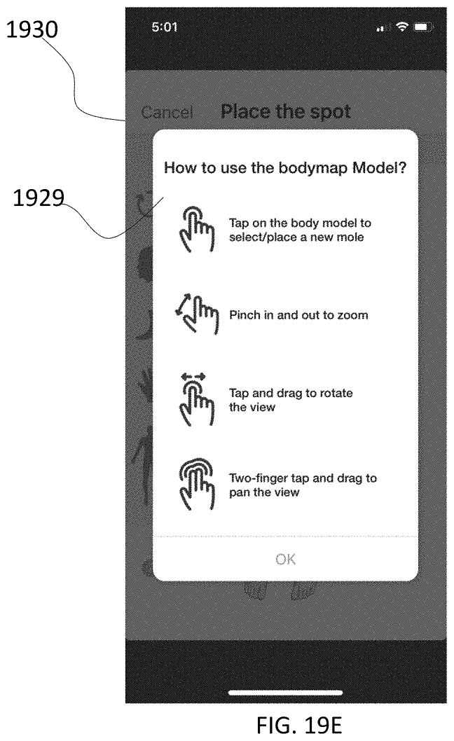

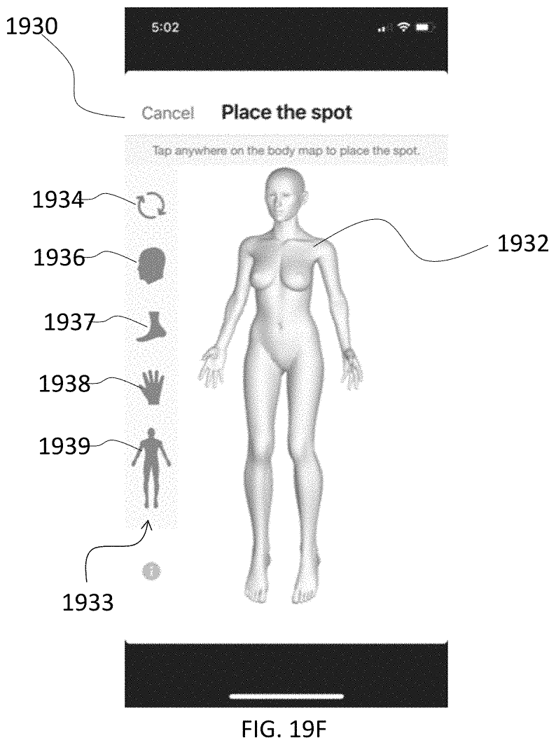

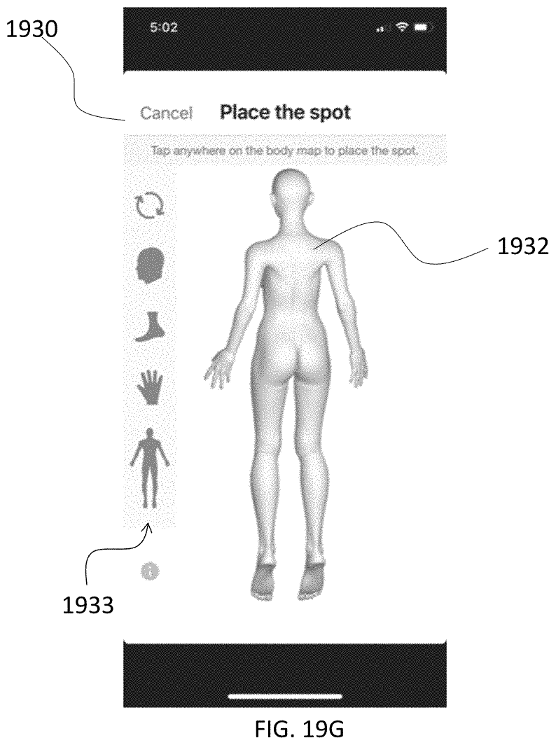

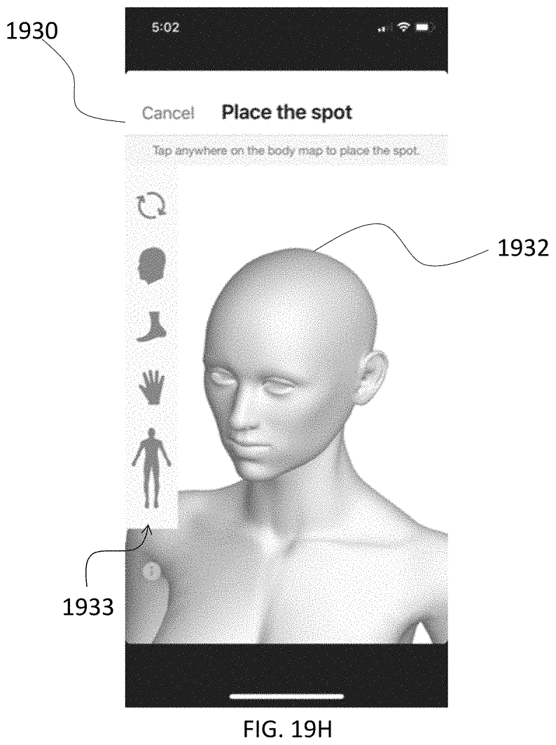

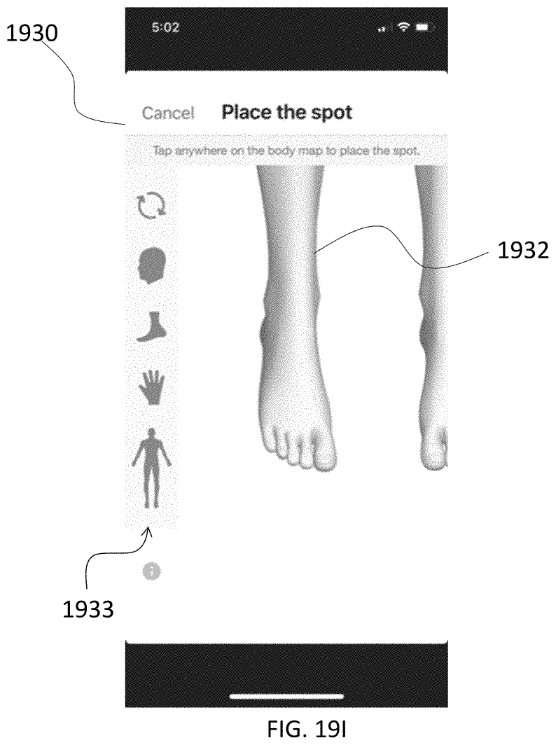

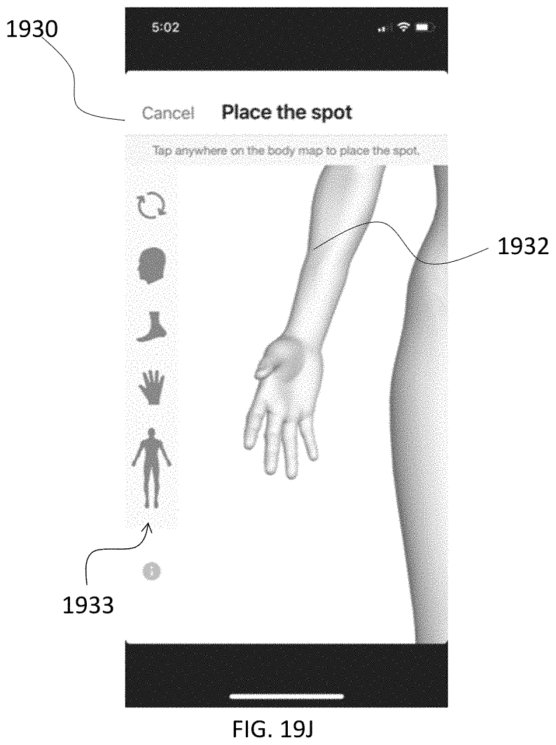

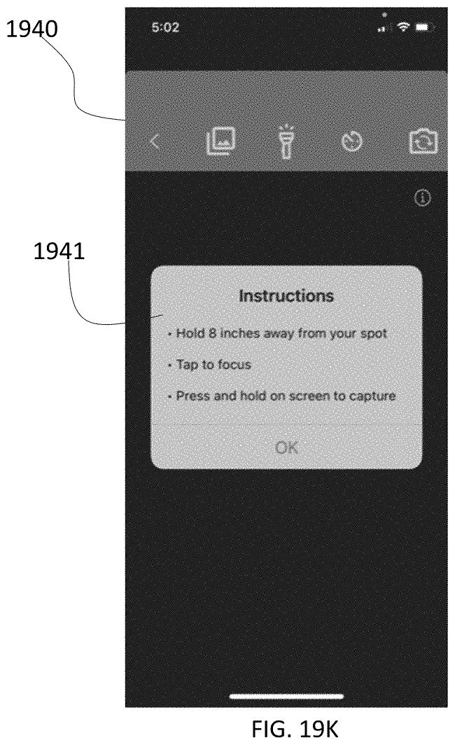

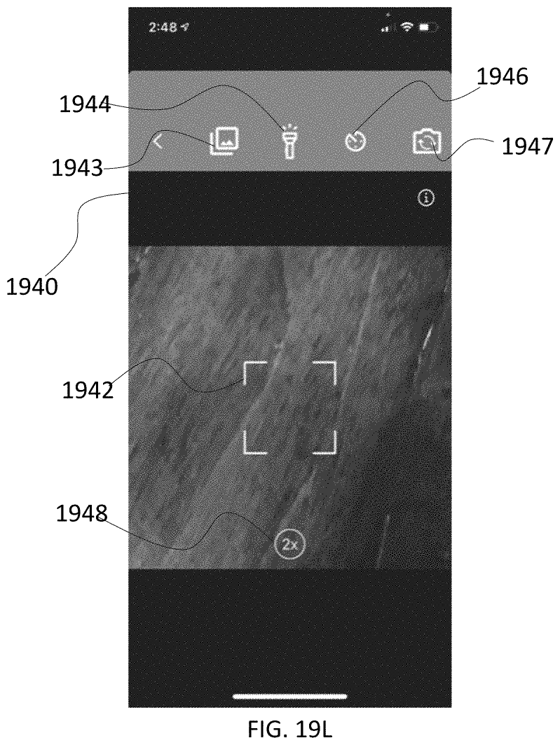

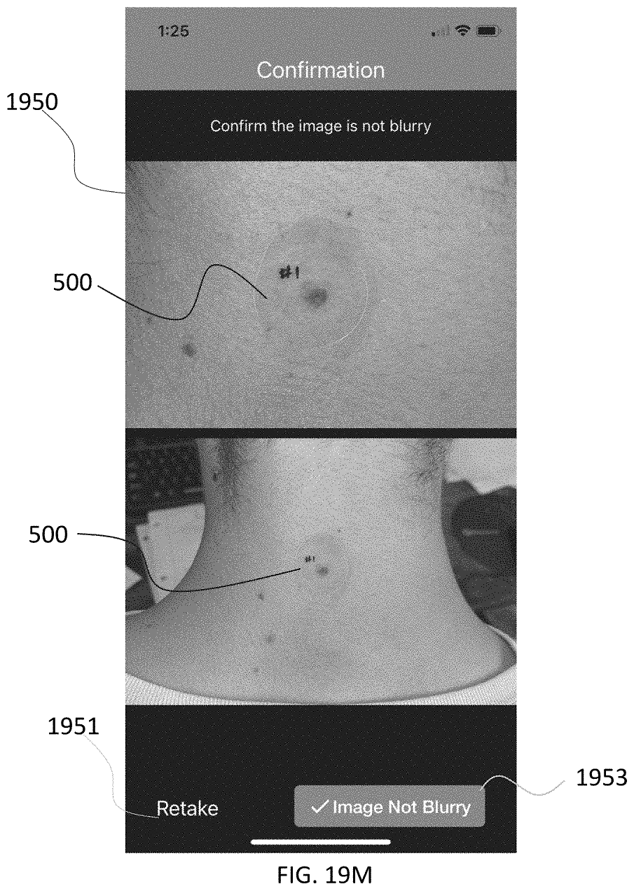

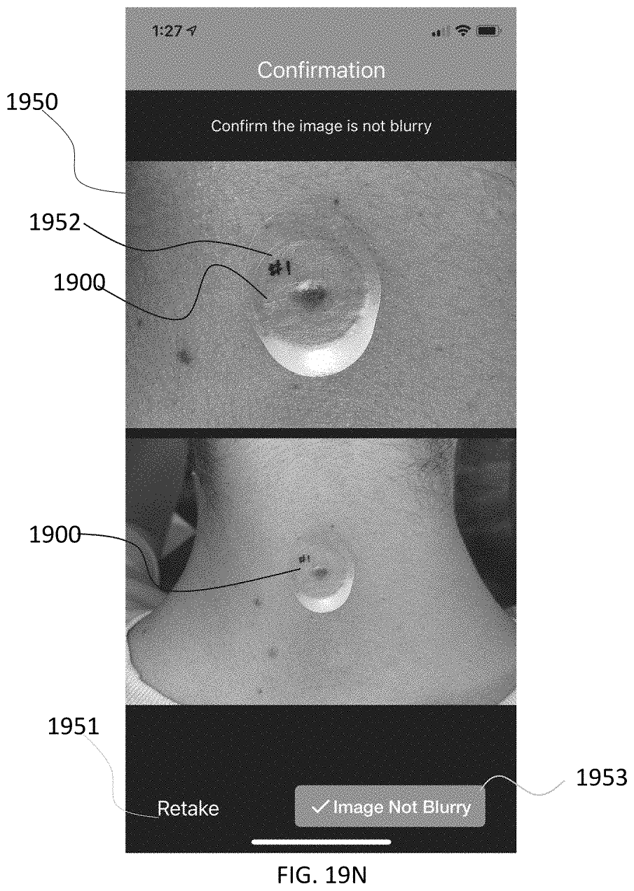

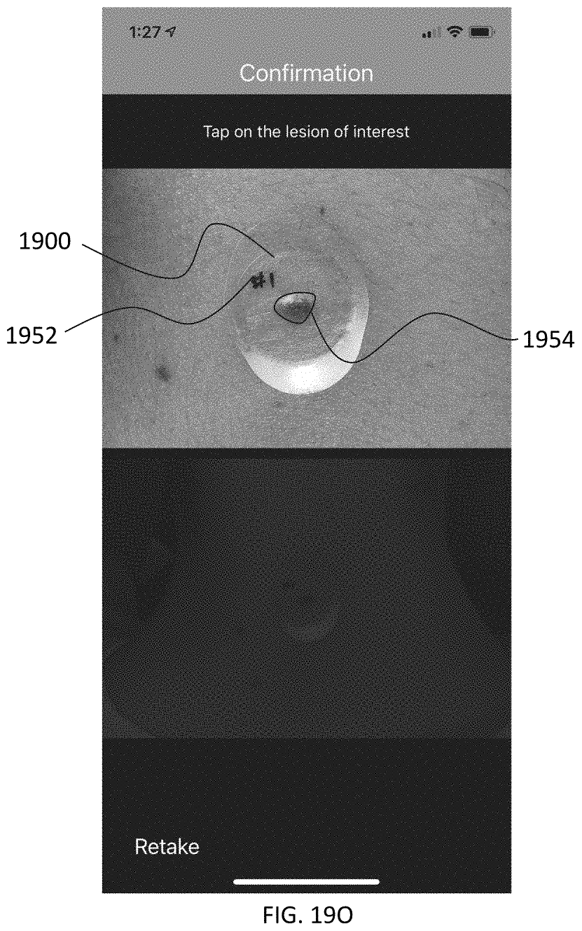

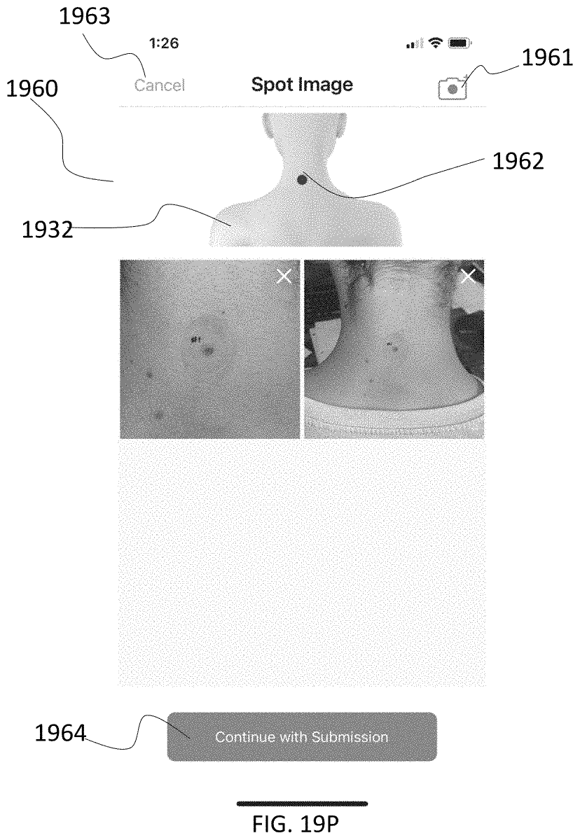

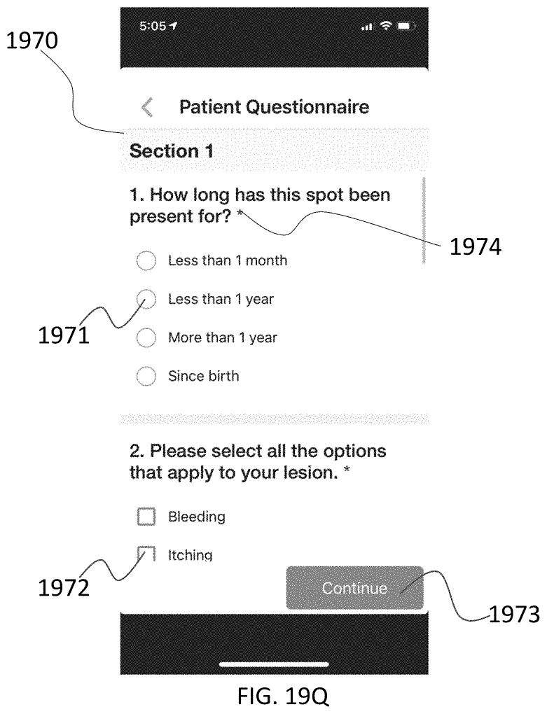

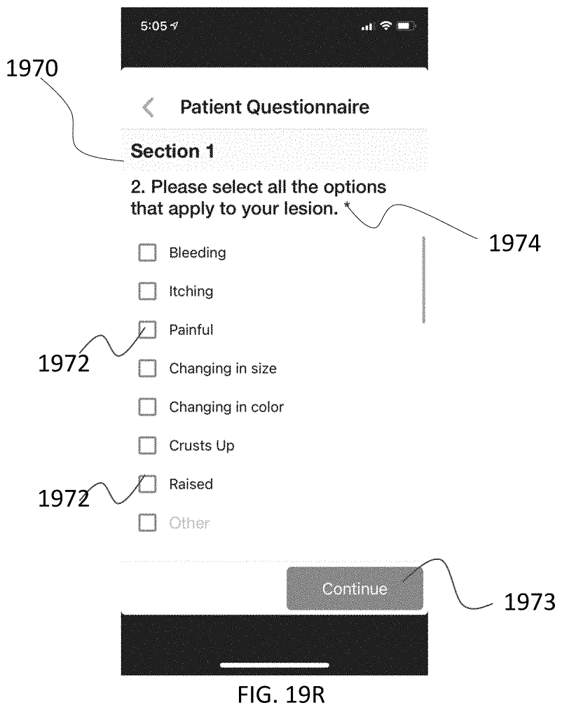

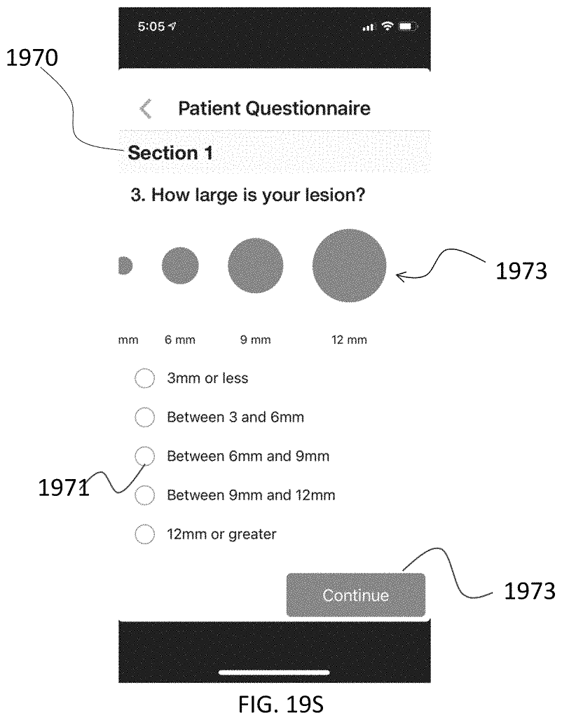

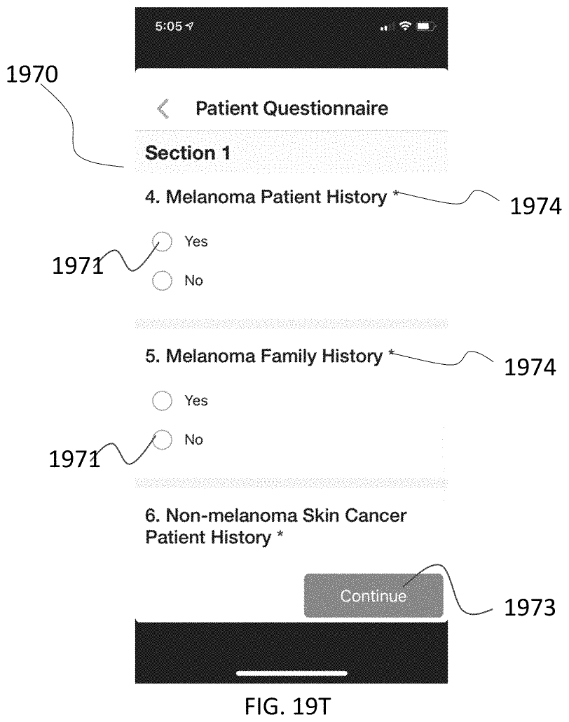

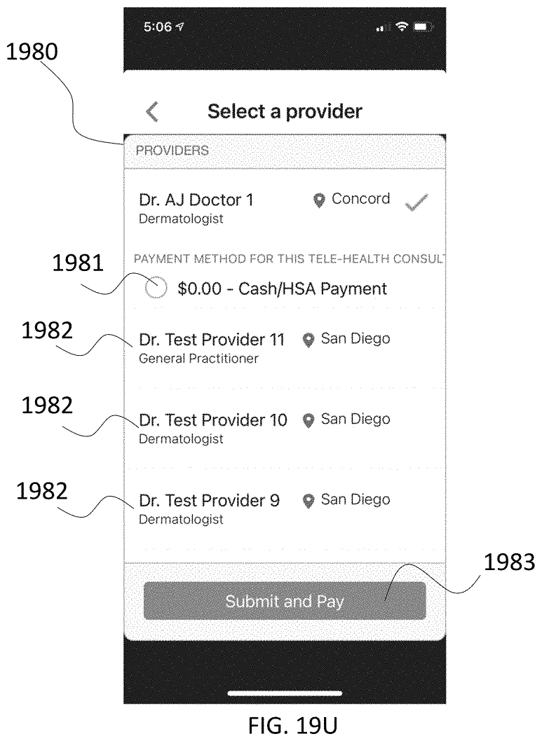

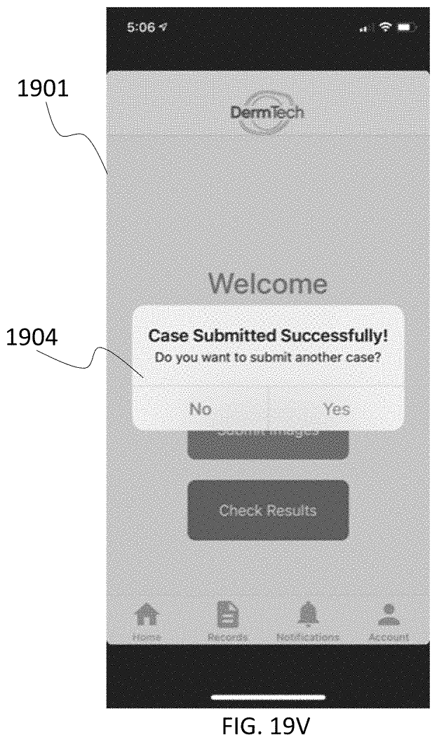

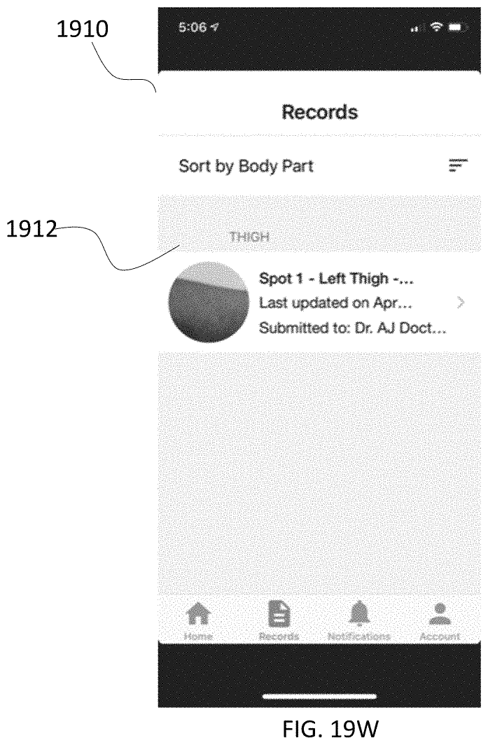

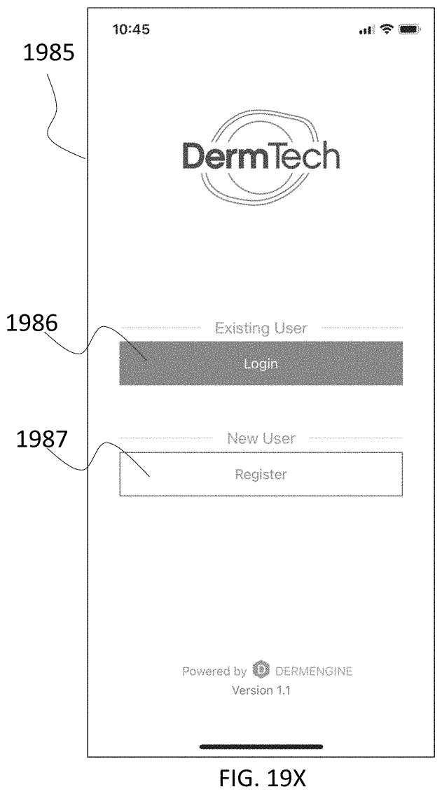

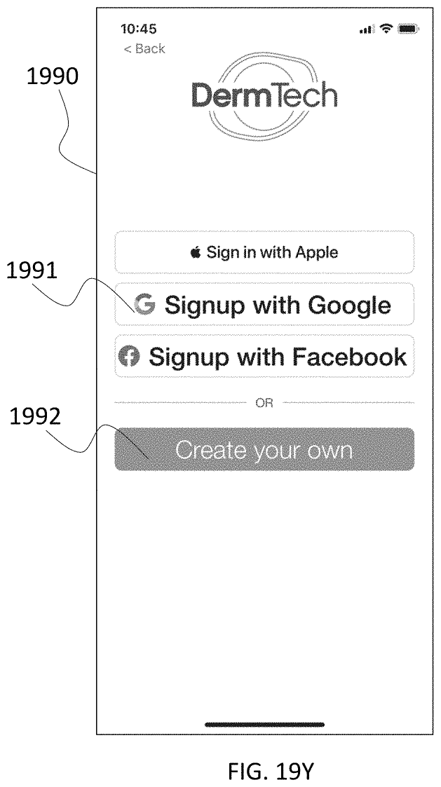

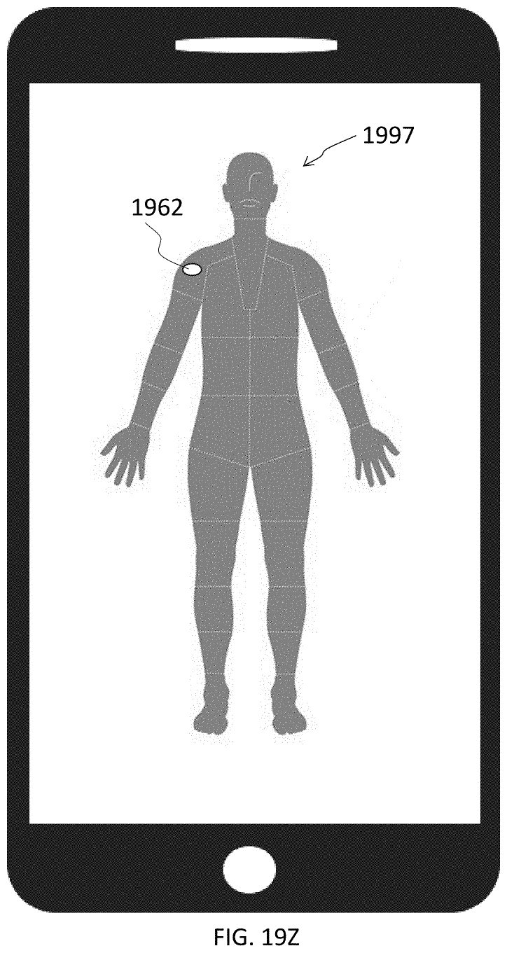

In some embodiments, the scanning of sample collectors further includes a sample validation step for automated acceptance/rejection of sample based on predetermined criteria. In some embodiments, the predetermined criteria may be identified by the scanning mechanism. In some embodiments, inconsistencies of the area to be tested or the presence of interfering substances may be criteria for automated acceptance or rejection of a sample. In some embodiments, image validation is based on images from a corresponding software application used to capture one or more images of a lesion (as depicted in FIGS. 19A-19Z).

In some embodiments, scanning of tissue samples further includes an image editing step. In some embodiment, image editing may be carried out by a visual inspection. In some embodiments, the image editing is based on the validation step. In some embodiments, image editing includes algorithmic evaluation of the digital images. Editing may include modifying the delineation to increase, decrease, shift, expand, contract, add to, delete from or otherwise modify the captured image. In some embodiments, image editing is based on images from a corresponding software application used to capture one or more images of a lesion (as depicted in FIGS. 19A-19Z).

Scanning of samples may further including additional image analysis, such as calculation of size, shape, color, or the like. In some embodiments, a scanning system reads indicia from the collector for identification and traceability.

B. Cutting of Sample Collectors

In some embodiments, cutting of each of the collected samples with an automated cutting system is based on an identified delineation to separate the cells of interest on the tissue sample collector from the remaining portion of the sample collector. Information collected during previous steps of the process may be used to process cutting of the samples.

Automated cutting may be performed via mechanical cutting, plasma cutting, or laser cutting. In some embodiments, laser cutting methods may include CO2, microjet, or fiber laser cutting.

In some embodiments, the laser cutter automatically cuts around the marked lesion areas, separating the lesion area from the surrounding area on each patch. Cutting may include vaporization, melt and blow, melt blow and burn, thermal stress cracking, scribing, cold cutting and burning stabilized laser cutting methods. In some embodiments, the cutting is performed based on identified markings on each of the one or more samples obtained from a computerized scanning of the array of collected samples. In some embodiments, the cutting is performed based on data obtained during the non-invasive adhesive sampling process.

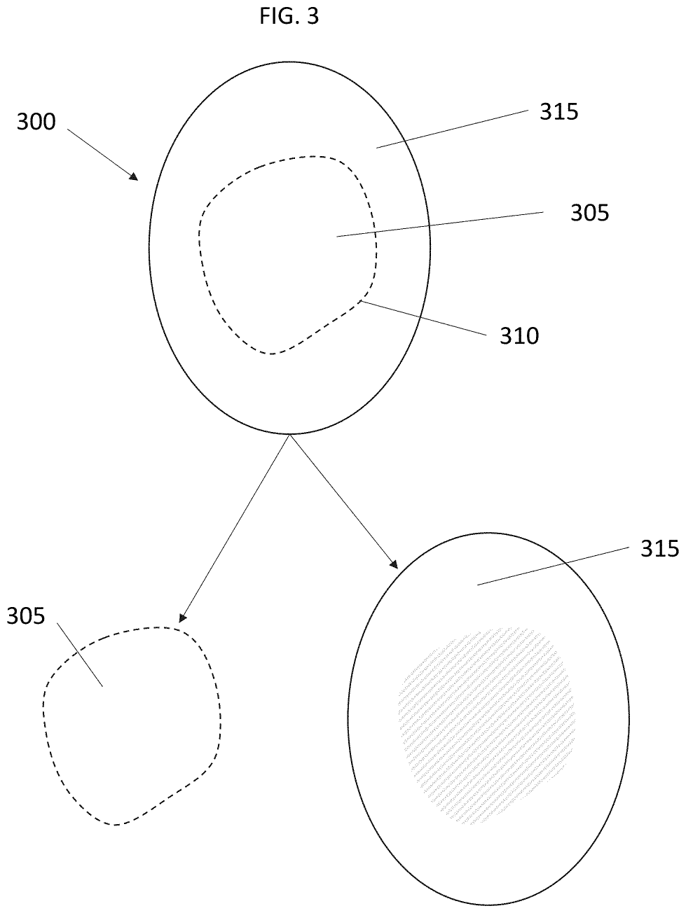

According to some embodiments, FIG. 3 depicts delineation, cutting, and separation of an area containing cells of interest 305 from a surrounding area 315 of a sample collector. In some embodiments, an adhesive patch 300 of a sample collector comprises an area containing cells of interest 305. In some embodiments, a delineation 310 between the cells of interest and the surrounding area is identified. In some embodiments, the adhesive patch 300 is then cut using the systems and methods as described herein. In some embodiments, the area of the sample collector comprising cells of interest 305 is separated from the surrounding area of the sample collector 315 after the patch 300 is cut along the border of the delineation 310.

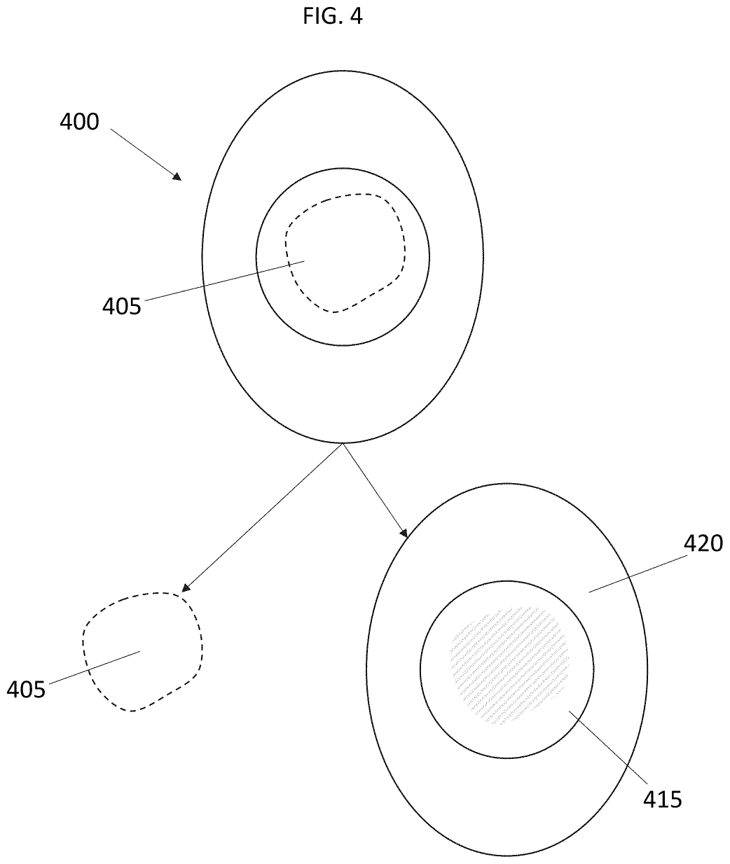

According to some embodiments, FIG. 4 depicts delineation, cutting, and separation of an area containing cells of interest 405 from a surrounding areas of a sample collector. In some embodiments, an adhesive patch 400 of a sample collector comprises a collection area 415 comprising an adhesive matrix and containing cells of interest 405 and a handling area 420. In some embodiments, the handling area 420 does not comprise an adhesive matrix. In some embodiments, delineation 410 between the cells of interest and the surrounding areas is identified. In some embodiments, the adhesive patch 400 is then cut using the systems and methods as described herein. In some embodiments, the area of the sample collector comprising cells of interest 405 is separated from the surrounding areas of the sample collector after the patch 400 is cut along the border of the delineation 410. In some embodiments, the remaining portion of the sample collector comprises portions of the collection area 415 which do not contain cells of interest and the handling area 420.

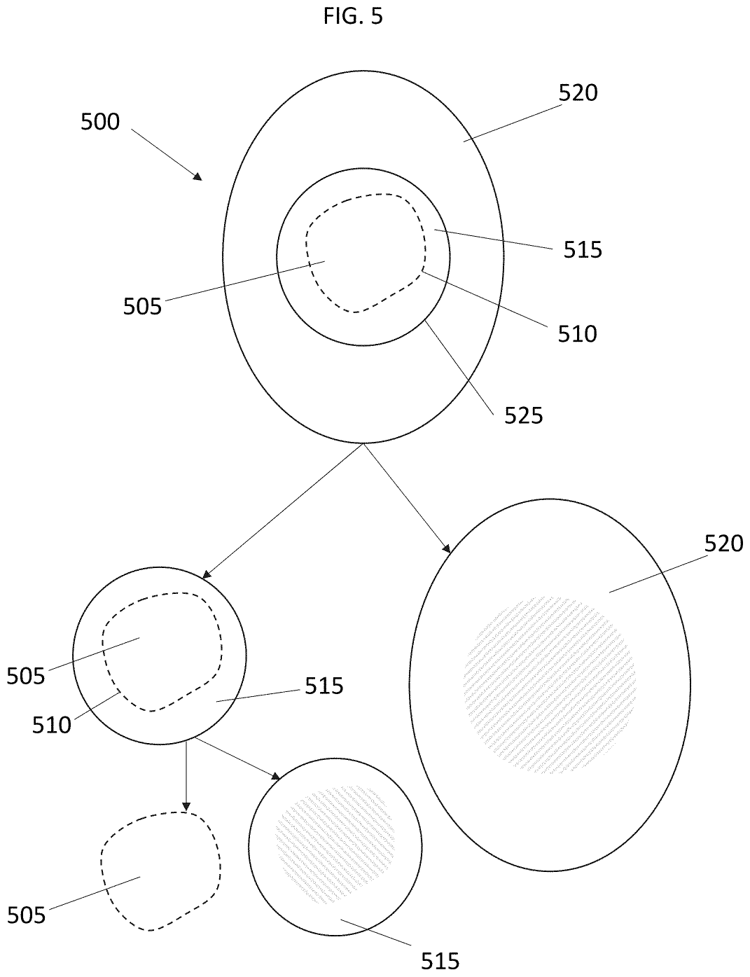

According to some embodiments, FIG. 5 depicts delineation, cutting, and separation of an area containing cells of interest 505 from a surrounding areas of a sample collector 500. In some embodiments, an adhesive patch 500 of a sample collector comprises a collection area 515 containing cells of interest 505 and a handling area 520. In some embodiments, the handling area 520 does not comprise an adhesive matrix. In some embodiments, delineation 510 between the cells of interest and the surrounding portions of the sample collector is identified. In some embodiments, a delineation 525 between the collection area 515 comprising an adhesive matrix and the handling area 520 is identified. In some embodiment, the adhesive patch 500 is then cut using the systems and methods as described herein. In some embodiment, the area comprising the adhesive matrix 515 is separated from the handling area 520 after the patch is cut along the border of delineation 525. In some embodiments, the area of the sample collector comprising cells of interest 505 is then separated from the portions of the collection area 515 which does not comprise cells of interest after the patch 500 is cut along the border of the delineation 510.

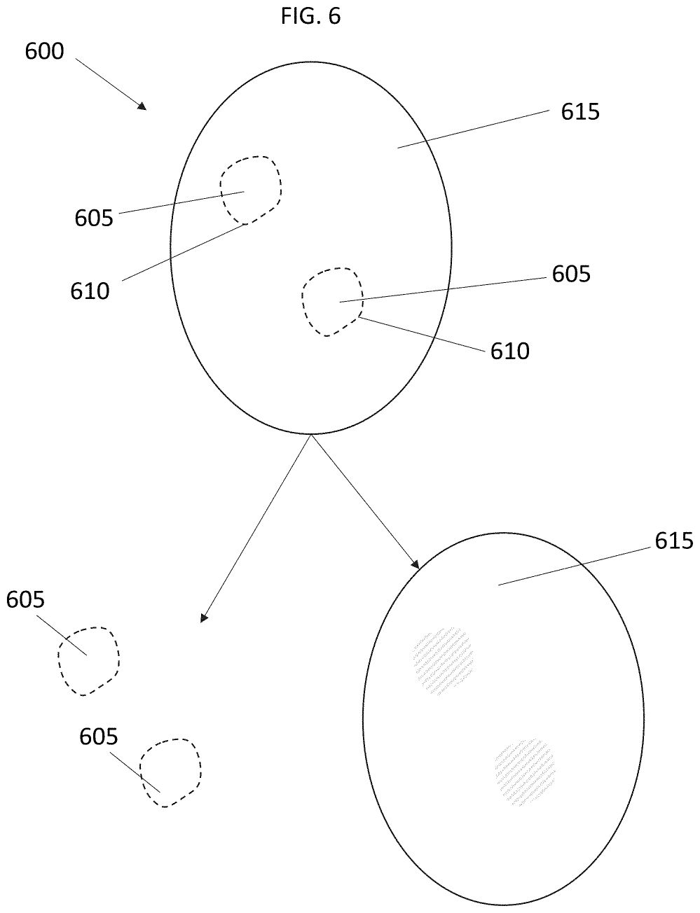

According to some embodiments, FIG. 6 depicts delineation, cutting, and separation of one or more areas containing cells of interest 605 from a surrounding area 615 of a sample collector. In some embodiments, an adhesive patch 600 of a sample collector comprises one or more an areas containing cells of interest 605. In some embodiments, delineations 610 between the areas containing cells of interest and the surrounding area is identified. In some embodiments, the adhesive patch 600 is then cut using the systems and methods as described herein. In some embodiments, the areas of the sample collector comprising cells of interest 605 are separated from the surrounding area of the sample collector 615 after the patch 600 is cut along the borders of the delineations 610.

As disclosed herein, delineations may be identified by demarcations provided on the sample collectors or adhesive patches. Delineations may be identified by scanning of the sample collector for areas containing cells of interest. In some embodiments, delineations correspond with borders of lesion areas to which the non-invasive tissue sample collector is applied.

C. Cutting Systems

Systems used to cut sample collectors containing cells of interest may comprise a mechanical cutting systems, a plasma cutting systems, or a laser cutting systems as disclosed herein. Mechanical cutting systems may include water-jet and die cutting systems. Plasma cutting systems may include inverter plasma cutting systems, plasma torch systems, and CNC (computer numerical control) cutting systems. In some embodiments, the laser cutting system is a laser engraving system. In some embodiments, a laser engraver system such as a GCC LaserPro brand model C18011 laser engraver is utilized for cutting samples from adhesive patches, as disclosed in some embodiments herein.

In some embodiments, a laser system is utilized to cut the tissue sample collector for segregation of an area of interest from a remaining portion of the sample collector. The laser system may comprise a laser capable of emitting a beam of light with enough energy to cut the material of the sample collector. The configuration of the laser may be further selected such that unintentional damage is not inflicted upon the tissue sample or underlying equipment of the system.

The laser beam or beam light may be emitted by a laser. The laser light may be emitted by a continuous wave laser. The laser light may be emitted by a pulsed laser. The laser light may be emitted by a gas laser, such as a helium-neon (HeNe) laser, an argon (Ar) laser, a krypton (Kr) laser, a xenon (Xe) ion laser, a nitrogen (N₂) laser, a carbon dioxide (CO₂) laser, a carbon monoxide (CO) laser, a transversely excited atmospheric (TEA) laser, or an excimer laser. For instance, the laser light may be emitted by an argon dimer (Ar₂) excimer laser, a krypton dimer (Kr₂) excimer laser, a fluorine dimer (F₂) excimer laser, a xenon dimer (Xe₂) excimer laser, an argon fluoride (ArF) excimer laser, a krypton chloride (KrCl) excimer laser, a krypton fluoride (KrF) excimer laser, a xenon bromide (XeBr) excimer laser, a xenon chloride (XeCl) excimer laser, or a xenon fluoride (XeF) excimer laser. The laser light may be emitted by a dye laser.

The laser light may be emitted by a metal-vapor laser, such as a helium-cadmium (HeCd) metal-vapor laser, a helium-mercury (HeHg) metal-vapor laser, a helium-selenium (HeSe) metal-vapor laser, a helium-silver (HeAg) metal-vapor laser, a strontium (Sr) metal-vapor laser, a neon-copper (NeCu) metal-vapor laser, a copper (Cu) metal-vapor laser, a gold (Au) metal-vapor laser, a manganese (Mn) metal-vapor, or a manganese chloride (MnCl₂) metal-vapor laser.

The laser light may be emitted by a solid-state laser, such as a ruby laser, a metal-doped crystal laser, or a metal-doped fiber laser. For instance, the laser light may be emitted by a neodymium-doped yttrium aluminum garnet (Nd:YAG) laser, a neodymium/chromium doped yttrium aluminum garnet (Nd/Cr:YAG) laser, an erbium-doped yttrium aluminum garnet (Er:YAG) laser, a neodymium-doped yttrium lithium fluoride (Nd:YLF) laser, a neodymium-doped yttrium orthovanadate (ND:YVO4) laser, a neodymium-doped yttrium calcium oxoborate (Nd:YCOB) laser, a neodymium glass (Nd:glass) laser, a titanium sapphire (Ti:sapphire) laser, a thulium-doped yttrium aluminum garnet (Tm:YAG) laser, a ytterbium-doped yttrium aluminum garnet (Yb:YAG) laser, a ytterbium-doped glass (Yt:glass) laser, a holmium yttrium aluminum garnet (Ho:YAG) laser, a chromium-doped zinc selenide (Cr:ZnSe) laser, a cerium-doped lithium strontium aluminum fluoride (Ce:LiSAF) laser, a cerium-doped lithium calcium aluminum fluoride (Ce:LiCAF) laser, a erbium-doped glass (Er:glass), an erbium-ytterbium-codoped glass (Er/Yt:glass) laser, a uranium-doped calcium fluoride (U:CaF₂) laser, or a samarium-doped calcium fluoride (Sm:CaF₂) laser.

The laser light may be emitted by a semiconductor laser or diode laser, such as a gallium nitride (GaN) laser, an indium gallium nitride (InGaN) laser, an aluminum gallium indium phosphide (AlGaInP) laser, an aluminum gallium arsenide (AlGaAs) laser, an indium gallium arsenic phosphide (InGaAsP) laser, a vertical cavity surface emitting laser (VCSEL), or a quantum cascade laser.

The laser light may be continuous wave laser light. The laser light may be pulsed laser light. The laser light may have a pulse length of at least 1 femtoseconds (fs), at least 2 fs, at least 3 fs, at least 4 fs, at least 5 fs, at least 6 fs, at least 7 fs, at least 8 fs, at least 9 fs, at least 10 fs, at least 20 fs, at least 30 fs, at least 40 fs, at least 50 fs, at least 60 fs, at least 70 fs, at least 80 fs, at least 90 fs, at least 100 fs, at least 200 fs, at least 300 fs, at least 400 fs, at least 500 fs, at least 600 fs, at least 700 fs, at least 800 fs, at least 900 fs, at least 1 picosecond (ps), at least 2 ps, at least 3 ps, at least 4 ps, at least 5 ps, at least 6 ps, at least 7 ps, at least 8 ps, at least 9 ps, at least 10 ps, at least 20 ps, at least 30 ps, at least 40 ps, at least 50 ps, at least 60 ps, at least 70 ps, at least 80 ps, at least 90 ps, at least 100 ps, at least 200 ps, at least 300 ps, at least 400 ps, at least 500 ps, at least 600 ps, at least 700 ps, at least 800 ps, at least 900 ps, at least 1 nanosecond (ns), at least 2 ns, at least 3 ns, at least 4 ns, at least 5 ns, at least 6 ns, at least 7 ns, at least 8 ns, at least 9 ns, at least 10 ns, at least 20 ns, at least 30 ns, at least 40 ns, at least 50 ns, at least 60 ns, at least 70 ns, at least 80 ns, at least 90 ns, at least 100 ns, at least 200 ns, at least 300 ns, at least 400 ns, at least 500 ns, at least 600 ns, at least 700 ns, at least 800 ns, at least 900 ns, at least 1,000 ns, or more, including increments therein. The laser light may have a pulse length of at most 1,000 ns, at most 900 ns, at most 800 ns, at most 700 ns, at most 600 ns, at most 500 ns, at most 400 ns, at most 300 ns, at most 200 ns, at most 100 ns, at most 90 ns, at most 80 ns, at most 70 ns, at most 60 ns, at most 50 ns, at most 40 ns, at most 30 ns, at most 20 ns, at most 10 ns, at most 9 ns, at most 8 ns, at most 7 ns, at most 6 ns, at most 5 ns, at most 4 ns, at most 3 ns, at most 2 ns, at most 1 ns, at most 900 ps, at most 800 ps, at most 700 ps, at most 600 ps, at most 500 ps, at most 400 ps, at most 300 ps, at most 200 ps, at most 100 ps, at most 90 ps, at most 80 ps, at most 70 ps, at most 60 ps, at most 50 ps, at most 40 ps, at most 30 ps, at most 20 ps, at most 10 ps, at most 9 ps, at most 8 ps, at most 7 ps, at most 6 ps, at most 5 ps, at most 4 ps, at most 3 ps, at most 2 ps, at most 1 ps, at most 900 fs, at most 800 fs, at most 700 fs, at most 600 fs, at most 500 fs, at most 400 fs, at most 300 fs, at most 200 fs, at most 100 fs, at most 90 fs, at most 80 fs, at most 70 fs, at most 60 fs, at most 50 fs, at most 40 fs, at most 30 fs, at most 20 fs, at most 10 fs, at most 9 fs, at most 8 fs, at most 7 fs, at most 6 fs, at most 5 fs, at most 4 fs, at most 3 fs, at most 2 fs, at most 1 fs, or less, including increments therein. The laser light may have a pulse length that is within a range defined by any two of the preceding values. For instance, the laser light may have a pulse length between 1 ns and 50 ns.

The laser light may have a repetition rate of at least 1 hertz (Hz), at least 2 Hz, at least 3 Hz, at least 4 Hz, at least 5 Hz, at least 6 Hz, at least 7 Hz, at least 8 Hz, at least 9 Hz, at least 10 Hz, at least 20 Hz, at least 30 Hz, at least 40 Hz, at least 50 Hz, at least 60 Hz, at least 70 Hz, at least 80 Hz, at least 90 Hz, at least 100 Hz, at least 200 Hz, at least 300 Hz, at least 400 Hz, at least 500 Hz, at least 600 Hz, at least 700 Hz, at least 800 Hz, at least 900 Hz, at least 1 kilohertz (kHz), at least 2 kHz, at least 3 kHz, at least 4 kHz, at least 5 kHz, at least 6 kHz, at least 7 kHz, at least 8 kHz, at least 9 kHz, at least 10 kHz, at least 20 kHz, at least 30 kHz, at least 40 kHz, at least 50 kHz, at least 60 kHz, at least 70 kHz, at least 80 kHz, at least 90 kHz, at least 100 kHz, at least 200 kHz, at least 300 kHz, at least 400 kHz, at least 500 kHz, at least 600 kHz, at least 700 kHz, at least 800 kHz, at least 900 kHz, at least 1 megahertz (MHz), at least 2 MHz, at least 3 MHz, at least 4 MHz, at least 5 MHz, at least 6 MHz, at least 7 MHz, at least 8 MHz, at least 9 MHz, at least 10 MHz, at least 20 MHz, at least 30 MHz, at least 40 MHz, at least 50 MHz, at least 60 MHz, at least 70 MHz, at least 80 MHz, at least 90 MHz, at least 100 MHz, at least 200 MHz, at least 300 MHz, at least 400 MHz, at least 500 MHz, at least 600 MHz, at least 700 MHz, at least 800 MHz, at least 900 MHz, at least 1,000 MHz, or more, including increments therein. The laser light may have a repetition rate of at most 1,000 MHz, at most 900 MHz, at most 800 MHz, at most 700 MHz, at most 600 MHz, at most 500 MHz, at most 400 MHz, at most 300 MHz, at most 200 MHz, at most 100 MHz, at most 90 MHz, at most 80 MHz, at most 70 MHz, at most 60 MHz, at most 50 MHz, at most 40 MHz, at most 30 MHz, at most 20 MHz, at most 10 MHz, at most 9 MHz, at most 8 MHz, at most 7 MHz, at most 6 MHz, at most 5 MHz, at most 4 MHz, at most 3 MHz, at most 2 MHz, at most 1 MHz, at most 900 kHz, at most 800 kHz, at most 700 kHz, at most 600 kHz, at most 500 kHz, at most 400 kHz, at most 300 kHz, at most 200 kHz, at most 100 kHz, at most 90 kHz, at most 80 kHz, at most 70 kHz, at most 60 kHz, at most 50 kHz, at most 40 kHz, at most 30 kHz, at most 20 kHz, at most 10 kHz, at most 9 kHz, at most 8 kHz, at most 7 kHz, at most 6 kHz, at most 5 kHz, at most 4 kHz, at most 3 kHz, at most 2 kHz, at most 1 kHz, at most 900 Hz, at most 800 Hz, at most 700 Hz, at most 600 Hz, at most 500 Hz, at most 400 Hz, at most 300 Hz, at most 200 Hz, at most 100 Hz, at most 90 Hz, at most 80 Hz, at most 70 Hz, at most 60 Hz, at most 50 Hz, at most 40 Hz, at most 30 Hz, at most 20 Hz, at most 10 Hz, at most 9 Hz, at most 8 Hz, at most 7 Hz, at most 6 Hz, at most 5 Hz, at most 4 Hz, at most 3 Hz, at most 2 Hz, at most 1 Hz, or less, including increments therein. The laser light may have a repetition rate that is within a range defined by any two of the preceding values.

The laser light may have a pulse energy of at least 1 nanojoule (nJ), at least 2 nJ, at least 3 nJ, at least 4 nJ, at least 5 nJ, at least 6 nJ, at least 7 nJ, at least 8 nJ, at least 9 nJ, at least 10 nJ, at least 20 nJ, at least 30 nJ, at least 40 nJ, at least 50 nJ, at least 60 nJ, at least 70 nJ, at least 80 nJ, at least 90 nJ, at least 100 nJ, at least 200 nJ, at least 300 nJ, at least 400 nJ, at least 500 nJ, at least 600 nJ, at least 700 nJ, at least 800 nJ, at least 900 nJ, at least 1 microjoule (μJ), at least 2 μJ, at least 3 μJ, at least 4 μJ, at least 5 μJ, at least 6 μJ, at least 7 μJ, at least 8 μJ, at least 9 μJ, at least 10 μJ, at least 20 μJ, at least 30 μJ, at least 40 μJ, at least 50 μJ, at least 60 μJ, at least 70 μJ, at least 80 μJ, at least 90 μJ, at least 100 μJ, at least 200 μJ, at least 300 μJ, at least 400 μJ, at least 500 μJ, at least 600 μJ, at least 700 μJ, at least 800 μJ, at least 900 μJ, a least 1 millijoule (mJ), at least 2 mJ, at least 3 mJ, at least 4 mJ, at least 5 mJ, at least 6 mJ, at least 7 mJ, at least 8 mJ, at least 9 mJ, at least 10 mJ, at least 20 mJ, at least 30 mJ, at least 40 mJ, at least 50 mJ, at least 60 mJ, at least 70 mJ, at least 80 mJ, at least 90 mJ, at least 100 mJ, at least 200 mJ, at least 300 mJ, at least 400 mJ, at least 500 mJ, at least 600 mJ, at least 700 mJ, at least 800 mJ, at least 900 mJ, a least 1 Joule (J), or more, including increments therein. The laser light may have a pulse energy of at most 1 J, at most 900 mJ, at most 800 mJ, at most 700 mJ, at most 600 mJ, at most 500 mJ, at most 400 mJ, at most 300 mJ, at most 200 mJ, at most 100 mJ, at most 90 mJ, at most 80 mJ, at most 70 mJ, at most 60 mJ, at most 50 mJ, at most 40 mJ, at most 30 mJ, at most 20 mJ, at most 10 mJ, at most 9 mJ, at most 8 mJ, at most 7 mJ, at most 6 mJ, at most 5 mJ, at most 4 mJ, at most 3 mJ, at most 2 mJ, at most 1 mJ, at most 900 μJ, at most 800 μJ, at most 700 μJ, at most 600 μJ, at most 500 μJ, at most 400 μJ, at most 300 μJ, at most 200 μJ, at most 100 μJ, at most 90 μJ, at most 80 μJ, at most 70 μJ, at most 60 μJ, at most 50 μJ, at most 40 μJ, at most 30 μJ, at most 20 μJ, at most 10 μJ, at most 9 μJ, at most 8 μJ, at most 7 μJ, at most 6 μJ, at most 5 μJ, at most 4 μJ, at most 3 μJ, at most 2 μJ, at most 1 μJ, at most 900 nJ, at most 800 nJ, at most 700 nJ, at most 600 nJ, at most 500 nJ, at most 400 nJ, at most 300 nJ, at most 200 nJ, at most 100 nJ, at most 90 nJ, at most 80 nJ, at most 70 nJ, at most 60 nJ, at most 50 nJ, at most 40 nJ, at most 30 nJ, at most 20 nJ, at most 10 nJ, at most 9 nJ, at most 8 nJ, at most 7 nJ, at most 6 nJ, at most 5 nJ, at most 4 nJ, at most 3 nJ, at most 2 nJ, at most 1 nJ, or less, including increments therein. The laser light may have a pulse energy that is within a range defined by any two of the preceding values. For instance, the laser light may have a pulse energy between 100 mJ and 500 mJ.

The laser light may have an average power of at least 1 microwatt (μV), at least 2 μW, at least 3 μW, at least 4 μW, at least 5 μW, at least 6 μW, at least 7 μW, at least 8 μW, at least 9 μW, at least 10 μW, at least 20 μW, at least 30 μW, at least 40 μW, at least 50 μW, at least 60 μW, at least 70 μW, at least 80 μW, at least 90 μW, at least 100 μW, at least 200 μW, at least 300 μW, at least 400 μW, at least 500 μW, at least 600 μW, at least 700 μW, at least 800 μW, at least 900 μW, at least 1 milliwatt (mW), at least 2 mW, at least 3 mW, at least 4 mW, at least 5 mW, at least 6 mW, at least 7 mW, at least 8 mW, at least 9 mW, at least 10 mW, at least 20 mW, at least 30 mW, at least 40 mW, at least 50 mW, at least 60 mW, at least 70 mW, at least 80 mW, at least 90 mW, at least 100 mW, at least 200 mW, at least 300 mW, at least 400 mW, at least 500 mW, at least 600 mW, at least 700 mW, at least 800 mW, at least 900 mW, at least 1 watt (W), at least 2 W, at least 3 W, at least 4 W, at least 5 W, at least 6 W, at least 7 W, at least 8 W, at least 9 W, at least 10 W, at least 20 W, at least 30 W, at least 40 W, at least 50 W, at least 60 W, at least 70 W, at least 80 W, at least 90 W, at least 100 W, at least 200 W, at least 300 W, at least 400 W, at least 500 W, at least 600 W, at least 700 W, at least 800 W, at least 900 W, at least 1,000 W, or more, including increments therein. The laser light may have an average power of at most 1,000 W, at most 900 W, at most 800 W, at most 700 W, at most 600 W, at most 500 W, at most 400 W, at most 300 W, at most 200 W, at most 100 W, at most 90 W, at most 80 W, at most 70 W, at most 60 W, at most 50 W, at most 40 W, at most 30 W, at most 20 W, at most 10 W, at most 9 W, at most 8 W, at most 7 W, at most 6 W, at most 5 W, at most 4 W, at most 3 W, at most 2 W, at most 1 W, at most 900 mW, at most 800 mW, at most 700 mW, at most 600 mW, at most 500 mW, at most 400 mW, at most 300 mW, at most 200 mW, at most 100 mW, at most 90 mW, at most 80 mW, at most 70 mW, at most 60 mW, at most 50 mW, at most 40 mW, at most 30 mW, at most 20 mW, at most 10 mW, at most 9 mW, at most 8 mW, at most 7 mW, at most 6 mW, at most 5 mW, at most 4 mW, at most 3 mW, at most 2 mW, at most 1 mW, at most 900 μW, at most 800 μW, at most 700 μW, at most 600 μW, at most 500 μW, at most 400 μW, at most 300 μW, at most 200 μW, at most 100 μW, at most 90 μW, at most 80 μW, at most 70 μW, at most 60 μW, at most 50 μW, at most 40 μW, at most 30 μW, at most 20 μW, at most 10 μW, at most 9 μW, at most 8 μW, at most 7 μW, at most 6 μW, at most 5 μW, at most 4 μW, at most 3 μW, at most 2 μW, at most 1 μW, or more, including increments therein. The laser light may have a power that is within a range defined by any two of the preceding values.

The laser light may comprise a wavelength in the ultraviolet (UV), visible, or infrared (IR) portions of the electromagnetic spectrum. The laser light may comprise a wavelength of at least 100 nanometers (nm), at least 110 nm, at least 120 nm, at least 130 nm, at least 140 nm, at least 150 nm, at least 160 nm, at least 170 nm, at least 180 nm, at least 190 nm, at least 200 nm, at least 210 nm, at least 220 nm, at least 230 nm, at least 240 nm, at least 250 nm, at least 260 nm, at least 270 nm, at least 280 nm, at least 290 nm, at least 300 nm, at least 310 nm, at least 320 nm, at least 330 nm, at least 340 nm, at least 350 nm, at least 360 nm, at least 370 nm, at least 380 nm, at least 390 nm, at least 400 nm, at least 410 nm, at least 420 nm, at least 430 nm, at least 440 nm, at least 450 nm, at least 460 nm, at least 470 nm, at least 480 nm, at least 490 nm, at least 500 nm, at least 510 nm, at least 520 nm, at least 530 nm, at least 540 nm, at least 550 nm, at least 560 nm, at least 570 nm, at least 580 nm, at least 590 nm, at least 600 nm, at least 610 nm, at least 620 nm, at least 630 nm, at least 640 nm, at least 650 nm, at least 660 nm, at least 670 nm, at least 680 nm, at least 690 nm, at least 700 nm, at least 710 nm, at least 720 nm, at least 730 nm, at least 740 nm, at least 750 nm, at least 760 nm, at least 770 nm, at least 780 nm, at least 790 nm, at least 800 nm, at least 810 nm, at least 820 nm, at least 830 nm, at least 840 nm, at least 850 nm, at least 860 nm, at least 870 nm, at least 880 nm, at least 890 nm, at least 900 nm, at least 910 nm, at least 920 nm, at least 930 nm, at least 940 nm, at least 950 nm, at least 960 nm, at least 970 nm, at least 980 nm, at least 990 nm, at least 1,000 nm, at least 1,010 nm, at least 1,020 nm, at least 1,030 nm, at least 1,040 nm, at least 1,050 nm, at least 1,060 nm, at least 1,070 nm, at least 1,080 nm, at least 1,090 nm, at least 1,100 nm, at least 1,110 nm, at least 1,120 nm, at least 1,130 nm, at least 1,140 nm, at least 1,150 nm, at least 1,160 nm, at least 1,170 nm, at least 1,180 nm, at least 1,190 nm, at least 1,200 nm, at least 1,210 nm, at least 1,220 nm, at least 1,230 nm, at least 1,240 nm, at least 1,250 nm, at least 1,260 nm, at least 1,270 nm, at least 1,280 nm, at least 1,290 nm, at least 1,300 nm, at least 1,310 nm, at least 1,320 nm, at least 1,330 nm, at least 1,340 nm, at least 1,350 nm, at least 1,360 nm, at least 1,370 nm, at least 1,380 nm, at least 1,390 nm, at least 1,400 nm, or more, including increments therein. The laser light may comprise a wavelength of at most 1,400 nm, at most 1,390 nm, at most 1,380 nm, at most 1,370 n, at most 1,360 nm, at most 1,350 nm, at most 1,340 nm, at most 1,330 nm, at most 1,320 nm, at most 1,310 nm, at most 1,300 nm, at most 1,290 nm, at most 1,280 nm, at most 1,270 n, at most 1,260 nm, at most 1,250 nm, at most 1,240 nm, at most 1,230 nm, at most 1,220 nm, at most 1,210 nm, at most 1,200 nm, at most 1,190 nm, at most 1,180 nm, at most 1,170 n, at most 1,160 nm, at most 1,150 nm, at most 1,140 nm, at most 1,130 nm, at most 1,120 nm, at most 1,110 nm, at most 1,100 nm, at most 1,090 nm, at most 1,080 nm, at most 1,070 n, at most 1,060 nm, at most 1,050 nm, at most 1,040 nm, at most 1,030 nm, at most 1,020 nm, at most 1,010 nm, at most 1,000 nm, at most 990 nm, at most 980 nm, at most 970 nm, at most 960 nm, at most 950 nm, at most 940 nm, at most 930 nm, at most 920 nm, at most 910 nm, at most 900 nm, at most 890 nm, at most 880 nm, at most 870 nm, at most 860 nm, at most 850 nm, at most 840 nm, at most 830 nm, at most 820 nm, at most 810 nm, at most 800 nm, at most 790 nm, at most 780 nm, at most 770 nm, at most 760 nm, at most 750 nm, at most 740 nm, at most 730 nm, at most 720 nm, at most 710 nm, at most 700 nm, at most 690 nm, at most 680 nm, at most 670 nm, at most 660 nm, at most 650 nm, at most 640 nm, at most 630 nm, at most 620 nm, at most 610 nm, at most 600 nm, at most 590 nm, at most 580 nm, at most 570 nm, at most 560 nm, at most 550 nm, at most 540 nm, at most 530 nm, at most 520 nm, at most 510 nm, at most 500 nm, at most 490 nm, at most 480 nm, at most 470 nm, at most 460 nm, at most 450 nm, at most 440 nm, at most 430 nm, at most 420 nm, at most 410 nm, at most 400 nm, at most 390 nm, at most 380 nm, at most 370 nm, at most 360 nm, at most 350 nm, at most 340 nm, at most 330 nm, at most 320 nm, at most 310 nm, at most 300 nm, at most 290 nm, at most 280 nm, at most 270 nm, at most 260 nm, at most 250 nm, at most 240 nm, at most 230 nm, at most 220 nm, at most 210 nm, at most 200 nm, at most 190 nm, at most 180 nm, at most 170 nm, at most 160 nm, at most 150 nm, at most 140 nm, at most 130 nm, at most 120 nm, at most 110 nm, at most 100 nm, or less, including increments therein. The laser light may comprise a wavelength that is within a range defined by any two of the preceding values.

The laser light may have a bandwidth of at least 0.001 nm, at least 0.002 nm, at least 0.003 nm, at least 0.004 nm, at least 0.005 nm, at least 0.006 nm, at least 0.007 nm, at least 0.008 nm, at least 0.009 nm, at least 0.01 nm, at least 0.02 nm, at least 0.03 nm, at least 0.04 nm, at least 0.05 nm, at least 0.06 nm, at least 0.07 nm, at least 0.08 nm, at least 0.09 nm, at least 0.1 nm, at least 0.2 nm, at least 0.3 nm, at least 0.4 nm, at least 0.5 nm, at least 0.6 nm, at least 0.7 nm, at least 0.8 nm, at least 0.9 nm, at least 1 nm, at least 2 nm, at least 3 nm, at least 4 nm, at least 5 nm, at least 6 nm, at least 7 nm, at least 8 nm, at least 9 nm, at least 10 nm, at least 20 nm, at least 30 nm, at least 40 nm, at least 50 nm, at least 60 nm, at least 70 nm, at least 80 nm, at least 90 nm, at least 100 nm, or more, including increments therein. The laser light may have a bandwidth of at most 100 nm, at most 90 nm, at most 80 nm, at most 70 nm, at most 60 nm, at most 50 nm, at most 40 nm, at most 30 nm, at most 20 nm, at most 10 nm, at most 9 nm, at most 8 nm, at most 7 nm, at most 6 nm, at most 5 nm, at most 4 nm, at most 3 nm, at most 2 nm, at most 1 nm, at most 0.9 nm, at most 0.8 nm, at most 0.7 nm, at most 0.6 nm, at most 0.5 nm, at most 0.4 nm, at most 0.3 nm, at most 0.2 nm, at most 0.1 nm, at most 0.09 nm, at most 0.08 nm, at most 0.07 nm, at most 0.06 nm, at most 0.05 nm, at most 0.04 nm, at most 0.03 nm, at most 0.02 nm, at most 0.01 nm, at most 0.009 nm, at most 0.008 nm, at most 0.007 nm, at most 0.006 nm, at most 0.005 nm, at most 0.004 nm, at most 0.003 nm, at most 0.002 nm, at most 0.001 nm, or less, including increments therein. The laser light may have a bandwidth that is within a range defined by any two of the preceding values.

The laser light may have a diameter (for instance, as measured by a Rayleigh beam width, full width at half maximum, 1/e2 width, second moment width, knife-edge width, D86 width, or any other measure of beam diameter) of at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1 mm, at least 2 mm, at least 3 mm, at least 4 mm, at least 5 mm, at least 6 mm, at least 7 mm, at least 8 mm, at least 9 mm, at least 10 mm, at least 20 mm, at least 30 mm, at least 40 mm, at least 50 mm, at least 60 mm, at least 70 mm, at least 80 mm, at least 90 mm, at least 100 mm, or more, including increments therein. The first light may have a diameter of at most 100 mm, at most 90 mm, at most 80 mm, at most 70 mm, at most 60 mm, at most 50 mm, at most 40 mm, at most 30 mm, at most 20 mm, at most 10 mm, at most 9 mm, at most 8 mm, at most 7 mm, at most 6 mm, at most 5 mm, at most 4 mm, at most 3 mm, at most 2 mm, at most 1 mm, at most 0.9 mm, at most 0.8 mm, at most 0.7 mm, at most 0.6 mm, at most 0.5 mm, at most 0.4 mm, at most 0.3 mm, at most 0.2 mm, at most 0.1 mm, or less, including increments therein. The laser light may have a diameter that is within a range defined by any two of the preceding values.

### III. SOFTWARE APPLICATION

In some embodiments, a software application loaded onto a user device is utilized to correspond images of a lesion or area of interest to samples being processed by the systems and methods herein. In some embodiments, the location of the lesion captured by the software application is utilized to verify a border of the lesion. In some embodiments, the images of the lesion captured by the software application are transmitted to the cutting and/or scanning systems to accurate cut an adhesive type sample collector at the border of the lesion of which the sample is being collected from.

With reference to FIGS. 19A-19Y, exemplary graphical user interface/user experience (GUI/UX) designs are depicted. In some embodiments, the GUI/UX facilitates evaluation of a skin lesion, affected area, or region affected by a skin condition. In some embodiments, the GUI/UX facilitates evaluation of a skin lesion, affected area, or region affected by a skin condition via a software application. In some embodiments, the GUI/UX facilitates evaluation of a skin lesion, affected area, or region affected by a skin condition via a web-based application.

The following will be given with reference to a device that detects inputs on a touch-sensitive surface display. In some embodiments, the device detects contact with the touch-sensitive display at locations that correspond to respective locations on the display. In this way, user inputs detected by the device on the touch-sensitive display are used by the device to manipulate the user interface on the display. In some embodiments, the device includes one or more contact intensity sensors for detecting intensity of contacts on a touch-sensitive display. In some embodiments, the device comprises one or more tactile output generators generating tactile outputs for a user of device. It should be understood that similar methods are, optionally, used for other user interfaces described herein.

Additionally, while the following examples are given primarily with reference to finger inputs (e.g., finger contacts, finger tap gestures, finger swipe gestures, etc.), it should be understood that, in some embodiments, one or more of the finger inputs are replaced with input from another input device (e.g., a mouse based input or a stylus input), or input of another type, on the same device (e.g., a button press). For example, a swipe gesture is, optionally, replaced with a mouse click (e.g., instead of a contact) followed by movement of the cursor along the path of the swipe (e.g., instead of movement of the contact). As another example, a tap gesture is, optionally, replaced with a mouse click while the cursor is located over the location of the tap gesture (e.g., instead of detection of the contact followed by ceasing to detect the contact). Similarly, when multiple user inputs are simultaneously detected, it should be understood that multiple computer mice are, optionally, used simultaneously, or a mouse and finger contacts are, optionally, used simultaneously.

In some embodiments, a predefined set of functions that are performed through a touchscreen and/or a touchpad. In some embodiments, functions are performed using one or more input devices, such as a mouse and keyboard. In some embodiments, functions include navigation between user interfaces. In some embodiments, the touchpad, when touched by the user, navigates device to a main, home, or root menu from any user interface that is displayed on the device. In some embodiments, other embodiments, the menu button is a physical push button or other physical input control device instead of a touchpad or touchscreen input. In some embodiments, the electronic device is in communication with a separate remote control through which it receives user inputs (e.g., the remote control includes a touch-sensitive surface or a touchscreen through which a user interacts with the electronic device).

FIG. 19A, illustrates a home screen user interface 1901 on a device with a touch-sensitive display, according to some. In some embodiments, one or more navigational objects 1905 are provided for navigation between user interfaces. In some embodiments, the one or more navigation objects comprise one or more virtual buttons which are selectable by a user. In some embodiments, one or more navigational objects 1905 are provided on multiple user interfaces of the software application to facilitate navigation.

In some embodiments, the navigation objects 1905 include a “home” button 1906. The “home” button may be selected by a user to return to the home screen user interface 1901. In some embodiments, the navigation objects 1905 include a “records” button 1907. The “records” button 1907 may be selected by a user to navigate to the records user interface 1910. In some embodiments, the navigation object 1905 include a “notifications” button 1908. The “notifications” button 1908 may be selected by a user to navigate to a notifications user interface. In some embodiments, the notifications user interface displays alerts, notifications, and/or messages regarding status of an evaluation or lab results. In some embodiments, the navigation objects 1905 include an “account” button 1909. The “account” button 1909 may be selected by a user to navigate to the account user interface 1915.

In some embodiments, the home screen user interface 1901 comprises one or more user input objects. In some embodiments, the user input object comprises one or more virtual buttons which are selectable by a user. In some embodiments, the user input objects include a “submit images” button 1902. In some embodiments, the submit images button 1902 is selectable by a user to begin an image capture, mapping, and transmission procedure, as described herein. The image capture, mapping, and transmission procedure may be designed to capture images and information corresponding to a lesion or region of interest. Through a series of graphic user interfaces to present a user experience, data corresponding to a lesion or region of interest may be assembled into a submission package or case for submission to a physician for examination. In some embodiments, the user input objects include a “check results” button 1903. In some embodiments, the “check results” button is selectable to display results, including lab results or physician evaluations, from previously submitted evaluations.

FIG. 19B illustrates a records user interface 1910 on a device with a touch-sensitive display. In some embodiments, one or more navigational objects 1905 are provided on the records interface 1910 for navigation between user interfaces.

In some embodiments, the records user interface 1910 comprises one or more selectable radio buttons 1911. In some embodiments, the radio buttons are selectable to sort records of previously submitted evaluation packages in the records display window 1912. FIG. 19B illustrates a records display window 1912 prior to submission of any evaluation packages. FIG. 19W depicts a records display window 1912 after submission of an evaluation packages. In some embodiments, an evaluation package refers to the captured images of a region of interest affected by a skin condition, or a lesion, combined with submitted information used to describe the region of interest affected by a skin condition, or the lesion.

In some embodiments, the radio buttons 1911 allow a user to sort the evaluation package records by body part, by spot number, or by modified date. In some embodiments, only one radio button is selectable at a time. In some embodiments, the spot number refers to the order in which the evaluation package was submitted, i.e., a first evaluation package corresponding to images of a first lesion or skin condition and the corresponding data would be assigned spot number one, and a second evaluation package corresponding to images of a second lesion or skin condition and the corresponding data would be assigned spot number two. In some embodiments, wherein images and/or evaluation packages of the same lesion, affected area, or spot are captured at two different times, evaluation package submitted at the later date will be assigned the same spot number.

FIG. 19C, illustrates an account user interface 1915 on a device with a touch-sensitive display. In some embodiments, one or more navigational objects 1905 are provided on the account interface 1910 for navigation between user interfaces.

In some embodiments, the account interface comprises a user information window 1916. In some embodiments, the user information window displays the name of a user logged into the application. In some embodiments, the user information window displays a recorded email address of the user logged into the application. In some embodiments, the user information window is selectable. In some embodiments, selection of the user information window navigates the user to a page to edit user information.

In some embodiments, the account user interface comprises a selectable “log out” button 1917. In some embodiments, selection of the “log out” button 1917 exits the application, such that input of user information and/or a password is required to log into the application and access data associated with a user. In some embodiments, the log out button 1917 returns the user to a login user interface (1990 as depicted in FIG. 19X).

In some embodiments, the account user interface 1915 further comprises a selectable “settings” button 1918 for navigation to a settings user interface 1920. In some embodiments, the account user interface 1915 further comprises a selectable “licenses” button 1919 for displaying information of the licenses associated with the application. In some embodiments, the account user interface 1915 displays the installed version of the application. In some embodiments, the account user interface 1915 provides a selectable “version” button to navigate to a new interface for updated the version of the application. In some embodiments, a selectable “version” button opens a new software application loaded onto the device for updated the application. The software application for updating the skin evaluation software application may be an app store, e.g., Google Play.

FIG. 19D, illustrates a settings user interface 1920 on a device with a touch-sensitive display, according to some embodiments. In some embodiments, the setting user interface comprises one or more user input objects associated with security settings. In some embodiments, one or more selectable security setting buttons allow for toggling of security preferences. In some embodiments the one or more selectable security buttons comprise an “always keep me logged in” button 1921, a “always ask for credentials” button 1922, and/or a “keep me logged in for a day” button 1923. In some embodiments, an “always keep me logged in” button 1921 is selectable such that a user does not need to enter credentials upon opening the software application. In some embodiments, an “always ask for credentials” button 1922 is selectable such that a user will need to enter credentials upon every instance of opening the software application and/or upon every instance of locking the device which the software application is loaded on. In some embodiments, if the always ask for credentials button 1922 is selected the user will return to a login user interface (1985 as depicted in FIG. 19X) each time the application is closed, the device is locked, and/or the device is powered off. In some embodiments, a “keep me logged in for a day” button 1923 is selectable such that a user does not need to enter credentials for a duration of 24 hours after an instance of entering credentials to log into the application.

In some embodiments, the settings user interface comprises one or more user input objects associated with reminder settings. In some embodiments, a reminder toggle switch 1924 is provided to turn on or turn off notifications associated with reminders to submit evaluation packages for lesions or affected skin areas which should be monitored. In some embodiments, the notifications are device notifications (e.g., bubble or pop-up notifications), email notifications, phone notifications, and/or text notifications (e.g., SMS or MMS messages).

In some embodiments, the setting user interface comprises one or more user input objects associated with body map and image enhancement settings. In some embodiments, a body map model toggle switch 1925 is provided to toggle between an avatar having a two-dimension representation (1997 as depicted in FIG. 19Z) or a three-dimensional representation. In some embodiments, an image enhancement toggle switch 1926 is provided to turn on or off image enhancement features, such as automated image brightening. In some embodiments, an auto-crop images toggle switch 1927 is provided to turn on or off an auto-crop feature configured to automatically crop images one or more images of a lesion or affected region of skin after the images are captured.

In some embodiments, after selection of the “submit images” button on the home user interface 1901, location marker interface 1930 is displayed. In some embodiments, a gesture information user interface 1929 is presented as a user lands on the location marker interface 1929. In some embodiments, the gesture information user interface 1929 displays a window comprising the acceptable inputs for controlling a virtual avatar (1932 as depicted in FIGS. 19F-19J). In some embodiments, touchscreen gestures to manipulate a virtual avatar include tapping on the avatar to select a location of a lesion or affected skin region, pinching to zoom in or out of the avatar, taping and dragging to rotate the view of the avatar, and two-finger dragging to pan the view of the avatar. In some embodiments, tapping and dragging to rotate the view of the avatar is only available if the 3D model of the avatar is being utilized.

FIGS. 19F-19J depict an avatar user interface 1930, according to some embodiments. In some embodiments, the avatar user interface 1930 displays a virtual avatar 1932 to provide a representation of a human body. As discussed herein, the avatar 1932 may be manipulated and regions of the avatar may be selected to place a location where an affected skin area is being captured. In some embodiments, the avatar user interface 1930 includes one or more quick manipulation buttons 1933 for quickly manipulating the avatar and facilitate placement of a lesion location. In some embodiments, the manipulation buttons comprise a flip button 1934 to quickly flip the avatar 180 degrees about a front plane. In some embodiments, the manipulation buttons comprise a head zoom button 1936 to quickly zoom into the head region of the avatar. In some embodiments, the manipulation buttons comprise a feet zoom button 1937 to quickly zoom into region near the feet of the avatar. In some embodiments, the manipulation buttons comprise a hands zoom button 1938 to quickly zoom into region near the hands of the avatar. In some embodiments, the manipulation buttons comprise a full profile button 1939 to quickly zoom out to display the full body of the avatar.

FIG. 19F depicts a front-facing view of the avatar 1932, according to some embodiments. In some embodiments, this view is the default view of the location marker interface 1930. In some embodiments, a user can quickly switch to this view by selecting the full profile button 1939.

FIG. 19G depicts a rear-facing view of the avatar 1932, according to some embodiments. In some embodiments, a user can quickly switch to this view by selecting the full profile button 1939, then the flip button 1934. In some embodiment, a user can quickly switch to this view by selecting the full profile button 1939 twice.

FIG. 19H depicts a zoomed in view of the head of the avatar 1932, according to some embodiments. In some embodiments, a user can quickly switch to this view by selecting the head zoom button 1937.

FIG. 19I depicts a zoomed in view of a foot of the avatar 1932, according to some embodiments. In some embodiments, a user can quickly switch to this view by selecting the feet zoom button 1938.

FIG. 19J depicts a zoomed in view of a hand of the avatar 1932, according to some embodiments. In some embodiments, a user can quickly switch to this view by selecting the hand zoom button 1939.

After a location of an affected skin area is selected, an image capturing user interface 1940 is provided, according to some embodiments. FIG. 19K depicts an image capturing user interface 1940 and instruction window 1941, which provides instructions for the successful capture of image of the affected skin area, according to some embodiments. In some embodiments, the instructions direct a user to hold the device/camera about 8 inches away from the affected area, spot, or lesion; tap the touchscreen of the device to focus the image; and press and hold the touch screen to capture the image or images of the affected area. In some embodiments, the instruction window 1941 is displayed for a predetermined amount of time. In some embodiments, the instruction window 1941 is displayed until the user taps the screen.

FIG. 19L depicts an image capturing user interface 1940, according to some embodiments. In some embodiments, the image capturing user interface 1940 comprises a reticle 1942 to facilitate placement of the target spot within the frame of the image to be captured. In some embodiments, the image capturing user interface 1940 comprises an upload button 1943 to select an image which is stored on the device's memory for uploading to the software application. In some embodiments, the image capturing user interface 1940 comprises a light button 1944 to turn on the device's flashlight. In some embodiments, the image capturing user interface 1940 comprises a timer button 1946 for setting a timer to capture an image using the device. In some embodiments, the image capturing user interface 1940 comprises a camera flip button 1947 to flip between the rear- and front-facing cameras of the device. In some embodiments, the image capturing user interface 1940 comprises a zoom indicator 1948 for displaying the current zoom level of the camera. The zoom indicator 1948 may be selectable to adjust the zoom level of the camera.

FIGS. 19M and 19MM depict a confirmation user interface 1950, according to some embodiments. In some embodiments, FIG. 19MM depicts images captured using a camera at a higher zoom level than the images captured by a camera as depicted in FIG. 19M. In some embodiments, the confirmation user interface 1950 displays one or more images showing the affected skin area as captured. In some embodiments, the confirmation user interface 1950 displays an image showing the affected skin area which is zoomed in and cropped. In some embodiments, the confirmation user interface 1950 displays the zoomed in image next to the image captured without zoom or magnification. In some embodiments, the confirmation user interface 1950 comprises one or more selectable buttons. In some embodiments, the selectable buttons comprise a “retake” button 1951 provided to navigate back to the image capturing user interface 1940 if the user confirms that the captured images are blurry. In some embodiments, the selectable buttons comprise a “image not blurry” confirmation button 1953 provided to continue to the lesion highlighting procedure if the user confirms the captured images are not blurry. Automated or cloud-based verification of quality image captures may be used to verify that captured images are not blurry.

FIG. 19O depicts the confirmation user interface 1950 during a lesion highlighting procedure, according to some embodiments. In some embodiments, a user taps on the captured image to highlight the lesion of interest with an outline 1954. In some embodiments, outline 1954 is displayed a circle. In some embodiments, outline 1954 is displayed an oval, square, triangle, or other suitable geometric shape. If the user is satisfied with the quality of the image, they may select the “next” button to continue to an image review user interface 1955. In some embodiments, outline 1954 is automatically applied to automatically applied. In some embodiment, computer vision techniques are utilized to identify the lesion of interest and apply the outline 1954. In some embodiments, a user verifies the correct placement of the outline 1954 has been applied. In some embodiments, a user verifies the correct shape of the outline 1954 has been applied.

In some embodiments, images of the lesion or area of interest are captured with an adhesive sample collector 1900 applied. In some embodiments, the sample collector 1900 comprises one or more fiducials 1952 printed on a surface of the sample collector. In some embodiments, the fiducials provide a reference for an orientation and distance relative to the lesion. In some embodiments, the fiducials provide a reference for the distance or zoom level at which the image of the sample collector 1900 placed on the lesion was captured. In some embodiments, the images of the sample collector 1900 and one or more fiducials 1952 are transmitted to the scanning and cutting system by the software application. The captured images may provide references for verification of correct demarcation of the lesion on the sample collector 1900. In some embodiments, captured images of a sample collector having at least one fiducial are utilized to automate cutting along an outline 1954 automatically generated by the system. In some embodiments, an outline 1954 is generated by the software application and transmitted to the scanning and cutting system.

FIG. 19P depicts an image review user interface 1960, according to some embodiments. In some embodiments, the image review user interface 1960 displays the avatar 1932 with the lesion location 1962. In some embodiments, the image review user interface 1960 displays the images captured by the software application. In some embodiments, the image review user interface 1960 displays both the regular and zoomed in images captured by the software application. In some embodiments, if a user is not satisfied with the captured images a camera button 1961 is provided to reload the image capturing user interface 1940. In some embodiments, if a user is not satisfied with the marked lesion location 1962 or wants to quite the submission process, a “cancel” button 1963 is provided as part of the image review user interface 1960. If the user is satisfied with the captured image and the marked location 1962, the “continue with submission” button 1964 may be selected to provide a questionnaire user interface 1970.

FIGS. 19Q-19T depict a questionnaire user interface 1970, according to some embodiments. In some embodiments, the questionnaire user interface presents a series of questions to obtain information from a user corresponding to the lesion or affected area, of which the images were captured using the camera of the device. In some embodiments, the questionnaire user interface 1970 comprises one or more radial buttons 1971 for selecting a single answer to a presented question. In some embodiments, the questionnaire user interface 1970 comprises one or more checkboxes 1971 for possible selection of multiple answers to a presented question.

In some embodiments, the questionnaire user interface 1970 comprises a “continue” button 1973 for continuing on to the provider selection user interface 1980. In some embodiments, a designation 1974 is used to mark questions which are required before proceeding to the provider selection user interface 1980. In some embodiments, the designation comprises a red asterisk.

As depicted in FIG. 19S, one question presented by the questionnaire user interface 1970 may require a user to input the size of the lesion of interest. In some embodiments, the selectable sizes comprise 3 millimeters (mm) or less, 6 mm, 9 mm, and 12 mm or greater. In some embodiments, a size key 1975 is provided to facilitate estimation of the size of the lesion of interest. In some embodiments, the size key 1975 displays reference images which are to scale or a 1:1 of the size being identified.

FIG. 19U depicts a provider selection user interface 1980, according to some embodiments. In some embodiments, the provider selection user interface 1980 provides one or more selectable provider buttons 1982 for choosing eligible providers. In some embodiments, eligible providers are listed and/or sorted by distance to a user's location or selected location. In some embodiments, the software applications interfaces with an insurance provider network, as described herein, such that only providers compatible with the user's insurance are displayed. In some embodiments, a selected provider is displayed at the top of the selection user interface 1980. In some embodiments, the selection user interface 1980 comprises one or more radial buttons 1981 to allow a user to select a payment method. In some embodiments, the software applications interfaces with an insurance provider network, as described herein, and the cost of the selectable payment methods is displayed. In some embodiments, the selection user interface 1980 provides a “submit and pay” button 1983 for submission of the payment information, images, and data collected by the software application. Upon submission the home screen user interface 1901 is reloaded.

In some embodiments, with reference to FIG. 19V, the home screen user interface 1901 may provide a pop-up window 1904 confirming the submission of the images and information. In some embodiments, the pop-up window 1904 provides a question to inquire if the user wishes to submit another case.

FIG. 19X depicts a login user interface 1985, according to some embodiments. In some embodiments, the login user interface comprises a selectable “login” button 1986 and a selectable “register” button. In some embodiments, selection of the “login” button 1987 loads a user credential interface for a user to enter their credentials to log into the software application. In some embodiments, selection of the “register” button 1987 loads a user registration interface 1990.

FIG. 19Y depicts a registration user interface 1990, according to some embodiments. In some embodiments, the registration user interface 1990 comprises one or more selectable linked registration buttons 1991 for linking an existing user account with the software application. An existing user account may be a social networking account, an email account, or other database account. In some embodiments, the registration user interface 1990 comprises a selectable custom registration button 1992. In some embodiments, upon selection of the custom registration button 1992, the registration user interface 1990 provides fields for a user to create credentials for the software application.

### IV. ANALYSIS OF CELLS OF INTEREST

In some embodiments, methods described herein further comprise analyzing the separated cells of interest. In some embodiments, information collected in previous steps may be used to analyze the separated cells of interest.

In some embodiments, analyzing the cells of interest includes removing the separated lesion area of the collected samples from the platform. In some embodiments, removal of the separated areas of interest is performed automatically as the first step in the analysis.

In some embodiments, during analysis, isolated RNA from a collected skin sample is reverse transcribed into cDNA for amplification by PCR to enrich for target genes. The expression levels of these target genes are quantified by quantitative PCR in a gene expression test. A gene expression test provides information on a gene expression signature associated with a disease. A pigmented lesion assay is an exemplary gene expression test which measures the expression levels of target genes from RNA isolated using the adhesive skin sample collection kit.

For example, in some embodiments, the pigmented lesion assay provides objective information on a gene expression signature associated with melanoma. Melanoma marker genes such as LINC and PRAME may be targeted. This information can be used to help support a histopathologic diagnosis or to determine the need for a biopsy, thereby reducing unnecessary biopsy procedures. The development of invasive tumor properties is also controlled by gene expression; therefore, the pigmented lesion assay may also differentiate invasive melanoma from melanoma in situ as well as provide staging information. The identification of invasive melanoma with metastatic potential will direct treatments to only those who need it. Another gene expression assay may determine if a melanoma tumor has spread to the lymph nodes. This test can reduce the need for a sentinel lymph node surgery, which can be extensive, cause morbidity and has significant medical costs.

Gene expression analyses facilitate drug development by identifying drug targets and stratifying patients into groups that will maximize a drug response. In an exemplary embodiment, a skin sample collected from the face of a subject with lupus is isolated and utilized in a gene expression test to assess the expression of target genes indicated in lupus drug effects. This gene expression test can identify responders to therapy and identify new drug targets. The use of the adhesive tape allows for skin sample collection without the scarring that can occur with a biopsy.

In some embodiments, one or more polypeptides isolated from the used adhesive tape are detected and/or quantified. For example, in some embodiments, one or more polypeptides isolated from the used adhesive tape are detected and/or quantified using ELISA, immunohistochemistry, mass spectrometry, and/or absorbance measurement. In some embodiments, the sequence of DNA isolated from the used adhesive tape is determined using gene sequencing methods known to one of skill in the art.

In some embodiments, isolated RNA from a collected skin sample is reverse transcribed into cDNA, for example for amplification by PCR to enrich for target genes. The expression levels of these target genes are quantified by quantitative PCR in a gene expression test. In some embodiments, in combination with quantitative PCR, a software program performed on a computer is utilized to quantify RNA isolated from the collected skin sample. In some embodiments, a software program or module is utilized to relate a quantity of RNA from a skin sample to a gene expression signature, wherein the gene expression signature is associated with a disease such as melanoma. In some embodiments, a software program or module scores a sample based on gene expression levels. In some embodiments, the sample score is compared with a reference sample score to determine if there is a statistical significance between the gene expression signature and a disease.

In some embodiments, the one or more target genes comprise C6orf218, preferentially expressed antigen in melanoma (PRAME), IL-6, IL-8, IL-17A, IL-17C, IL-17F, IL-17RA, IL-17RC, IL-21, IL-22, IL-23A, IL-24, IL-26, TNF-α, TNF RSF1A, S100A7, S100A9, CCL20, CXCL1, CXCL5, LCN2, DEFB4A, or a combination thereof. In some embodiments, the one or more target genes comprise a target gene selected from Table 1.

TABLE 1

| Gene Symbol | Species | Gene Name |
|---|---|---|
| IL-23A | Human | interleukin 23, alpha subunit p19 |
| IL-17A | Human | interleukin 17A |
| IL-17C | Human | interleukin 17C |
| IL-17F | Human | interleukin 17F |
| TNF-α | Human | tumor necrosis factor |
| IL-17RA | Human | interleukin 17 receptor A |
| IL-17RC | Human | interleukin 17 receptor C |
| TNF RSF1A | Human | tumor necrosis factor receptor superfamily, |
| | | member 1A |
| IL-6 | Human | interleukin 6 (interferon, beta 2) |
| IL-8 | Human | interleukin 8 |
| IL-21 | Human | interleukin 21 |
| IL-22 | Human | interleukin 22 |
| IL-24 | Human | interleukin 24 |
| IL-26 | Human | interleukin 26 |
| S100A7 | Human | S100 calcium binding protein A7 |
| S100A9 | Human | S100 calcium binding protein A9 |
| CCL20 | Human | chemokine (C-C motif) ligand 20 |
| CXCL1 | Human | chemokine (C-X-C motif) ligand 1 |
| | | (melanoma growth stimulating activity, |
| | | alpha) |
| CXCL5 | Human | chemokine (C-X-C motif) ligand 5 |
| LCN2 | Human | lipocalin 2 |
| DEFB4A | Human | defensin, beta 4A |

In some embodiments, the one or more target genes comprise C6orf218, preferentially expressed antigen in melanoma (PRAME), or a combination thereof. In some cases, the one or more target genes comprise C6orf218. In other cases, the one or more target genes comprise preferentially expressed antigen in melanoma (PRAME).

In some embodiments, one or more target genes from the isolated RNA obtained from a collected skin sample are analyzed, in which the one or more target genes comprise at least one target gene selected from Table 1. In some embodiments, from about 1 to about 100, from about 1 to about 90, from about 1 to about 80, from about 1 to about 70, from about 1 to about 60, from about 1 to about 50, from about 1 to about 40, from about 1 to about 30, from about 1 to about 20, from about 5 to about 100, from about 5 to about 80, from about 5 to about 60, from about 5 to about 40, from about 5 to about 20, from about 10 to about 100, from about 10 to about 80, from about 10 to about 60, from about 10 to about 40, from about 20 to about 80, from about 20 to about 60, from about 20 to about 40, from about 30 to about 80, from about 30 to about 60, from about 40 to about 60, from about 2 to about 10, from about 2 to about 8, or from about 2 to about 6 target genes from the isolated RNA obtained from a collected skin sample are analyzed, in which the one or more target genes comprise at least one target gene selected from Table 1.

In some cases, about 1, 2, 3, 4, 5, 6, 7, 8, 9, 10, 11, 12, 13, 14, 15, 20, 25, 30, 40, 50 or more, including increments therein, target genes from the isolated RNA obtained from a collected skin sample are analyzed, in which the one or more target genes comprise at least one target gene selected from Table 1. In some cases, about 1 or more target genes from the isolated RNA obtained from a collected skin sample are analyzed, in which the one or more target genes comprise at least one target gene selected from Table 1. In some cases, about 2 or more target genes from the isolated RNA obtained from a collected skin sample are analyzed, in which the one or more target genes comprise at least one target gene selected from Table 1. In some cases, about 3 or more target genes from the isolated RNA obtained from a collected skin sample are analyzed, in which the one or more target genes comprise at least one target gene selected from Table 1. In some cases, about 4 or more target genes from the isolated RNA obtained from a collected skin sample are analyzed, in which the one or more target genes comprise at least one target gene selected from Table 1. In some cases, about 5 or more target genes from the isolated RNA obtained from a collected skin sample are analyzed, in which the one or more target genes comprise at least one target gene selected from Table 1. In some cases, about 6 or more target genes from the isolated RNA obtained from a collected skin sample are analyzed, in which the one or more target genes comprise at least one target gene selected from Table 1. In some cases, about 7 or more target genes from the isolated RNA obtained from a collected skin sample are analyzed, in which the one or more target genes comprise at least one target gene selected from Table 1. In some cases, about 8 or more target genes from the isolated RNA obtained from a collected skin sample are analyzed, in which the one or more target genes comprise at least one target gene selected from Table 1. In some cases, about 9 or more target genes from the isolated RNA obtained from a collected skin sample are analyzed, in which the one or more target genes comprise at least one target gene selected from Table 1. In some cases, about 10 or more target genes from the isolated RNA obtained from a collected skin sample are analyzed, in which the one or more target genes comprise at least one target gene selected from Table 1. In some cases, about 11 or more target genes from the isolated RNA obtained from a collected skin sample are analyzed, in which the one or more target genes comprise at least one target gene selected from Table 1. In some cases, about 12 or more target genes from the isolated RNA obtained from a collected skin sample are analyzed, in which the one or more target genes comprise at least one target gene selected from Table 1. In some cases, about 13 or more target genes from the isolated RNA obtained from a collected skin sample are analyzed, in which the one or more target genes comprise at least one target gene selected from Table 1. In some cases, about 14 or more target genes from the isolated RNA obtained from a collected skin sample are analyzed, in which the one or more target genes comprise at least one target gene selected from Table 1. In some cases, about 15 or more target genes from the isolated RNA obtained from a collected skin sample are analyzed, in which the one or more target genes comprise at least one target gene selected from Table 1. In some cases, about 20 or more target genes from the isolated RNA obtained from a collected skin sample are analyzed, in which the one or more target genes comprise at least one target gene selected from Table 1. In some cases, about 25 or more target genes from the isolated RNA obtained from a collected skin sample are analyzed, in which the one or more target genes comprise at least one target gene selected from Table 1. In some cases, about 30 or more target genes from the isolated RNA obtained from a collected skin sample are analyzed, in which the one or more target genes comprise at least one target gene selected from Table 1. In some cases, about 40 or more target genes from the isolated RNA obtained from a collected skin sample are analyzed, in which the one or more target genes comprise at least one target gene selected from Table 1. In some cases, about 50 or more target genes from the isolated RNA obtained from a collected skin sample are analyzed, in which the one or more target genes comprise at least one target gene selected from Table 1.

The subject matter described herein, including the gene expression tests and corresponding transmission of data, in certain aspects, are configured to be performed in one or more facilities at one or more locations. Facility locations are not limited by country and include any country or territory. Facility locations are not limited by country and include any country or territory. In some embodiments, one or more steps of the gene expression test are performed in a different country than another step of the gene expression test. In some embodiments, one or more steps of the gene expression test are performed in a different country than one or more steps of the tape stripping aspect. In some embodiments, one or more articles are transferred from one or more of the facilities to one or more different facilities for analysis or further analysis. An article includes, but is not limited to, one or more components of the skin sample collection kit, a used adhesive tape, isolated cellular material obtained from a used adhesive tape, processed cellular material, and/or data. Processed cellular material includes, but is not limited to, cDNA reverse transcribed from RNA, amplified RNA, and amplified cDNA. Data includes, but is not limited to, information regarding the expression level of one or more target genes, information regarding a gene expression signature, and information regarding a disease, such as melanoma. In some embodiments of the methods, devices, and systems described herein, the analysis is performed and a subsequent data transmission step will convey or transmit the results of the analysis. Information regarding a disease, includes, but is not limited to, identification of a disease state, likelihood of treatment success for a given disease state, identification of progression of a disease state (e.g., invasiveness of melanoma), and identification of a disease stage (e.g., melanoma stages 0, 1, 2, 3, or 4).

In some embodiments, methods and systems as described herein may be utilized for evaluation and diagnostic procedures comprising a broad array of molecular tests. The methods and systems described herein may be utilized to obtain nucleic acid samples from a subject. The samples may comprise samples from a lesion area of the skin, as discussed herein.

The collected samples may undergo nucleic acid extraction to provide a purified nucleic acid product to be analyzed. Analysis may include analyses of host RNA and DNA, host RNA only, host DNA only, microbiome DNA, microbiome RNA, or combinations thereof.

An RNA-based analysis may include a gene expression analysis. The analysis may be carried out by RT-qPCR, RNA sequencing, or microarray techniques.

A DNA-based analysis may include a mutation or SNP analysis (genetic), or methylation (epigenetic). The analysis may include qPCR, AS-PCR (allele specific PCR), or sequencing techniques, including NGS (next generation sequencing), WGS (whole genome sequencing), sanger, or other suitable sequencing techniques.

A DNA/RNA skin microbiome analysis may include analysis of abundance and species of microbes, determination of healthy or pathogenic microbes, skin health assessment, skin disease complications, or a combination thereof. Techniques for assessment of skin microbiome may include DMRs (differentially methylated regions). DMRs may include BS-MSP (bisulfate-methylation specific PCR), bisulfate sequencing, whole genome bisulfate sequencing, bisulfate sanger, or other suitable techniques.

The analyses of may be used in skin cancer diagnostic assays, such as melanoma PLA, melanoma nevome assays, and carcinoma assays (including BCC and SCC). The analyses may be used for skin cancer risk assessment such as a luminate mutation test or a gene expression based UV damage assessment. The analyses may be used for skin inflammation and companion diagnostics such as diagnostics of psoriasis, psoriatic arthritis, atopic dermatitis, atopic asthma, vitiligo, lupus, cutaneous T-cell lymphoma, alopecia areata, drug reactions, and other diagnostics. The analyses may be used for a skin health assessment, which may include a gene based assessment of skin condition or skin age, and assessment of skin microbiome flora.

A. Determination of Portions of a Sample Collector to be Analyzed

In some embodiments, the resultant sensitivity and specificity of the analyzed lesion area of a sample is improved compared to a process that analyzes both the lesion area component of the sample and non-lesion area of the sample. In some embodiments, non-lesion material can dilute a small number of target copy numbers in a larger sample volume potentially creating false negative test results.

### 1. Example 1: Analysis of a Separated Lesion Area

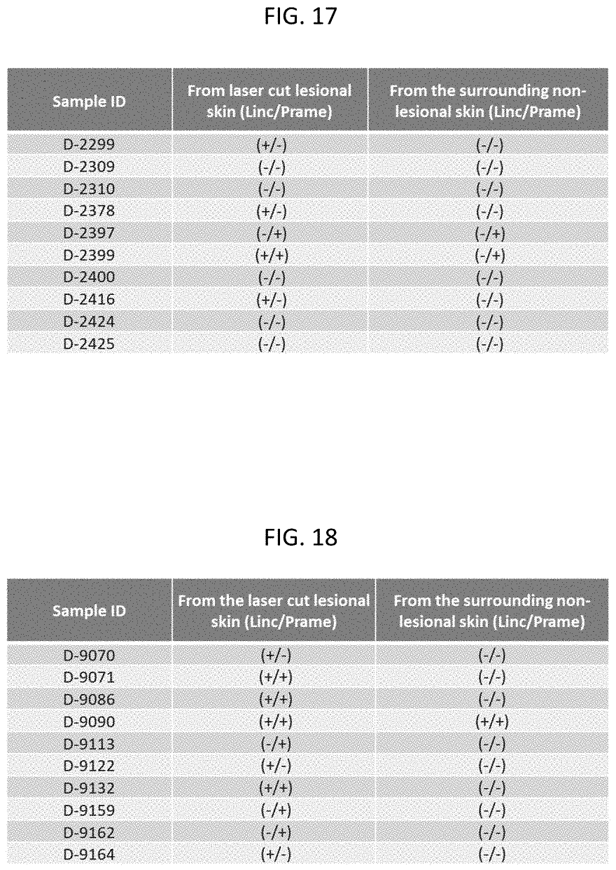

FIGS. 17 and 18 depict an analysis of results from a PLA (pigmented lesion assay) performed only on the lesion cells of interest cut and separated from the non-lesion surrounding area to improve sensitivity and specificity of the analysis, as compared to an analysis of the PLA results from the non-lesion surrounding area.

In some embodiments, melanoma marker genes such as LINC (Long intergenic non-coding RNA) and PRAME (Preferentially expressed antigen in melanoma) are targeted to diagnose the existence of melanoma. Approximately 93% of PLA results positive for both LINC and PRAME may be diagnosed histopathologically as melanoma.

The analysis provided by FIGS. 17 and 18 demonstrates the difference in gene expression between lesional and the surrounding non-lesional skins. As depicted in FIGS. 17 and 18, the “+” symbol denotes a detected gene expression and the “−” symbol denotes that the particular gene expression was not detected. It can be seen that the results from the surrounding samples would provide cells that would increase the probability of a false negative test result.

Although the example embodied by FIGS. 17 and 18 depict processes for PLA testing, other analyses that diagnose diseases and conditions using the non-invasive tissue collector may benefit from some type of automated cutting process/system.

### 2. Example 2: Analysis of Lesion Area and Surrounding Area

Smaller atopic or other inflammatory lesions may be sampled and processed using the methods described herein. However, in some examples, both the lesional cells and non-lesional (surrounding) cells may be tested and analyzed. Testing of both portions may allow for differentiation of a variety of processes that may be useful, for example, to differentiate inflammatory responses from underlying root cause responses (e.g., autoimmune disease). In some examples, two or more cuts may be required, based on a delineation of an inner portion of the tissue collector and a delineation of outer portion(s) of the tissue collector, allowing for differential analysis of the two different portions of the tissue collector (as depicted in FIGS. 5 and 6).

### V. COMPUTER SYSTEMS

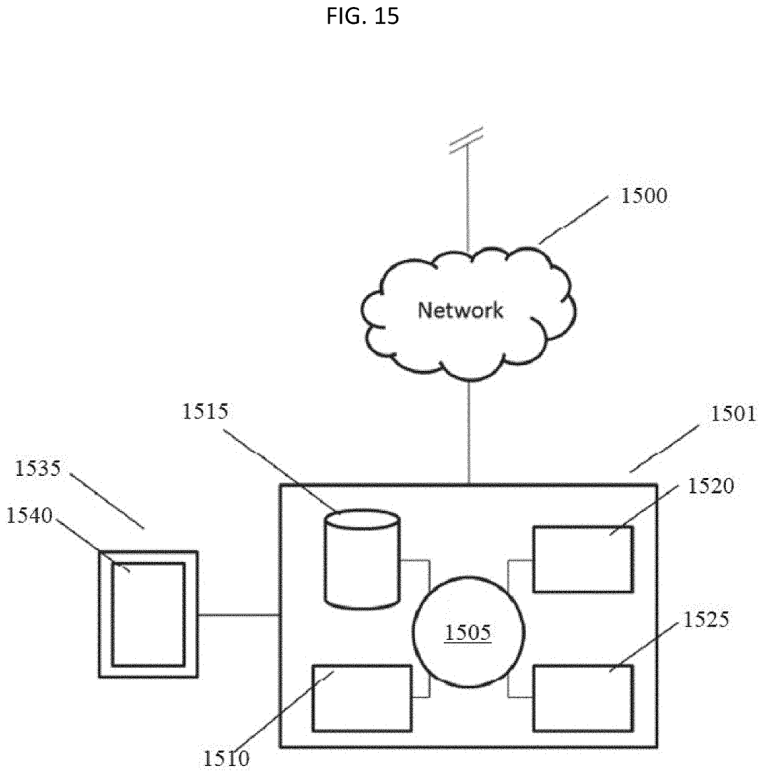

The present disclosure provides computer systems for implementing methods and devices of the present disclosure. FIG. 15 shows a computer system 1501 that is programmed or otherwise configured to operate any method or system described herein (such as any method of cutting a sample collector described herein). The computer system 1501 can regulate various aspects of the present disclosure. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine-readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, enact any of the methods for imparting color to a wearable ocular device as described herein.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### VI. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a,” “an,” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “portion” and “area” may be interchangeably user herein. The terms portion or area may refer to defined components of a sample collector. The terms portion or area may also refer to defined locations of a skin area or sample collector which contain or do not contain cells of interest.

The section headings used herein are for organizational purposes only and are not to be construed as limiting the subject matter described.

### VII. EXAMPLES

The following examples are included for illustrative purposes only and are not intended to limit the scope of the invention.

A. Exemplary Methods for Scanning and Cutting of Tissue Sample Collectors

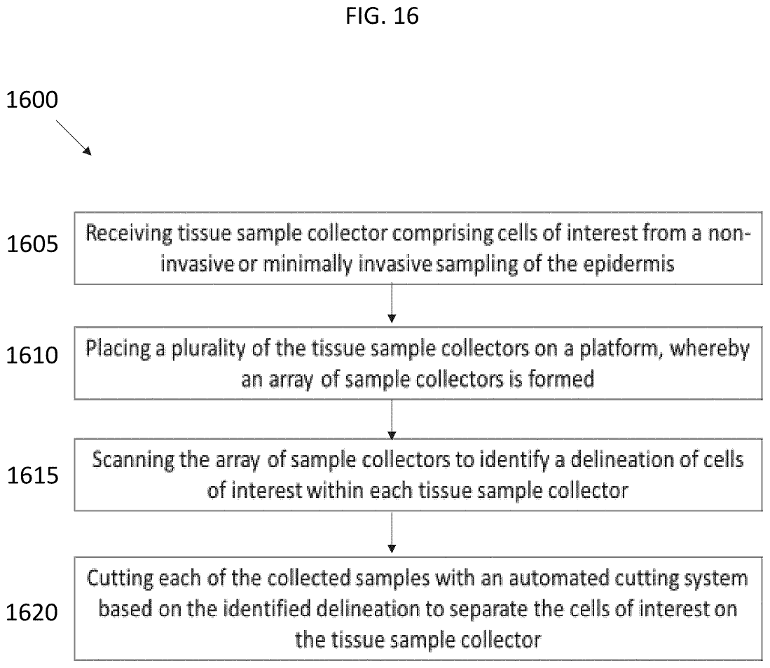

According to some embodiments, with reference to FIG. 16, an embodiment of a method 1600 for automated scanning and cutting of tissue samples is depicted. In some embodiments, the method comprises a first step 1605 of receiving one or more tissue sample collectors. In some embodiments, the tissue sample collectors comprise cells of interest. In some embodiments, the tissue samples are collected via non-invasive or minimally invasive sampling of the epidermis. In some embodiments, the sampling procedure is a non-invasive adhesive sampling procedure as described herein.

At a second step 1610, the sample collectors may be placed onto a platform of an automated tissue sample scanning and cutting system as described herein. In some embodiments, a plurality of sample collectors forms an array of sample collectors. In some embodiments, only a single sample collector or single sample may be provided on the platform. An array of sample collectors may be arranged in a linear or rectangular array as described herein.

At a third step 1615, the array of sample collectors may be scanned by the system. In some embodiments, the array is scanned to identify a delineation of cells of interest within each tissue sample of each sample collector. In some embodiments, the delineation is identified by one or more demarcations provided on the sample collectors. In some embodiments, the delineation is identified by a scanning procedure to identify a border between the cells of interest and a remaining portion of the sample collector.

At a fourth step, each sample of the sample collectors may be cut based on the identified delineation between the cells of interest and a remaining portion of the sample collector. In some embodiments, the samples are cut to separate the cells of interest from the remaining portion of the sample collector.

In some embodiments, a further step of separating the cut portions of the sample collector containing cells of interest from the remaining portion of the sample collector is carried out for each sample collector. In some embodiments, the remaining portions of the sample collector, not containing cells of interest, are discarded.

In some embodiments a further step of analyzing the cells of interest is carried out. Analyzation may comprise diagnosing of a skin condition based on an analysis of the cells of interest.

In some embodiments, each sample strip comprises four smart stickers, sample collectors, or adhesive patches. In some embodiments, if less/more than 4 smart stickers are received, all received all smart stickers will be rejected and documented in a database.

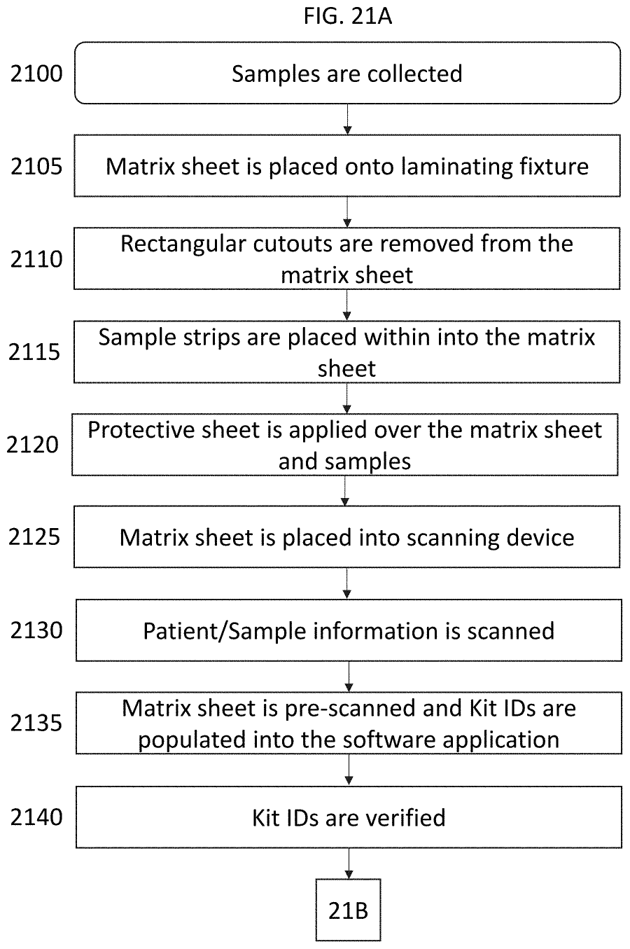

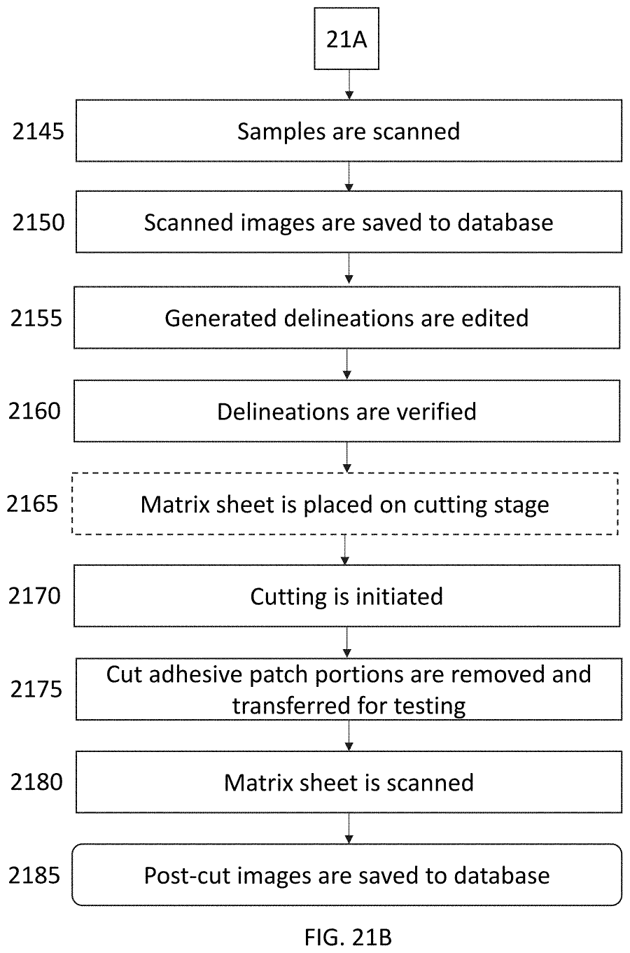

In some embodiments, specimen acceptance or rejection based on a quality not sufficient (QNS) criteria is determined by RNA yield. In some embodiments, samples are stored at room temperature. In some embodiments, samples are stable for up to 10 days from the collection date. In some embodiments, samples which or bloody and/or do not have lesions marked on all four patches are rejected. In some embodiment, samples with more than 6 non-vellus hairs having a terminus traversing lesion marked areas, 6 being the total for all 4 smart stickers, are rejected. With reference to FIGS. 21A and 21B, an exemplary method for scanning and cutting of adhesive patches is depicted.

1. Laminating Procedure

In some embodiments, at step 2100, sample strips are collected from collection kits. Sample strips may be collected from multiple collection kits for multiple patients. In some embodiments, at step sample strips are labeled and placed on a matrix sheet to be laser cut. In some embodiments, the matrix sheet comprises spaces to receive six sample strips. In some embodiments, at step 2105 the matrix sheet is placed is placed on a laminating fixture with a scored side up. In some embodiments, the samples are placed on the laminating fixture and registered to an indented corner with holes secured on locating or alignment pins.

In some embodiments, at step 2110, rectangle paper cutouts are removed to provide positions for the sample strips. In some embodiments, positions are labeled on the matrix as position #1 through position #6. In some embodiment, the paper cutouts are left in place if the positions are not utilized. In some embodiments, at step 2115, sample strips are placed in position #1 through #6 sequence on an adhesive sheet. In some embodiments, position #1 is in the top left corner and position #6 is in the bottom right corner. In some embodiments, each sample strip is aligned with the sample strip along one edge of the exposed adhesive and gently laid into place. In some embodiments, slight pressure is applied along the edges of the sample strip to permanently adhere the strip. In some embodiments, once the sample strip has been applied to the sample collector adhesive sheet, it cannot be removed.

In some embodiments, sample strips specimen collectors are provided as a bifold and information will not be on the back of the sample strip. In some embodiments, a bifold strip will be cut and the two sides will be taped together before applying the sample strip on the matrix sheet. Once all strips are in place, excess paper may be removed from the adhesive sheet.

In some embodiments, at step 2120, a clear protective sheet is applied to the exposed adhesive sheet. In some embodiments, the protective sheet comprises through holes to align with provided alignment pins. In some embodiments a laminating roller is utilized to adhere the protective cover to the adhesive matrix sheet. In some embodiments this completes a laminating procedure.

2. Scanning Procedure

In some embodiments, the matrix sheet, now comprising a plurality of sample strips and samples, which are laminated between an adhesive sheet and a protective cover, are removed from the laminating fixture. In some embodiments, at step 2125, the matrix sheet is laid onto a scanning apparatus. In some embodiments, through holes provided in the matrix sheet are aligned with alignment pins provided on the scanning device.

In some embodiments, at step 2130, information provided on a back side of the adhesive strips (opposite of the samples) is scanned first. In some embodiments, the samples and demarcations are scanned first. In some embodiments, the information and the samples are scanned simultaneously.

In some embodiments, once the matrix sheet is placed onto the scanning apparatus, a scanning and laser cutting software application is opened on a computing device. In some embodiments, a “Scan/Process” option is selected. In some embodiments, a scanning initiation GUI (2010 as depicted in FIG. 20A) is loaded.

In some embodiments, at step 2135, “PreScan Image” button is selected and the Kit ID fields provided in the GUI are automatically populated. In some embodiments, after population of the Kit ID numbers, a “Scan and Process” button is selected. In some embodiments, a window may appear to confirm the selection of “Scan Outline” or “Scan Centerline.” In some embodiments, a window displays the Kit IDs and their corresponding positions in the matrix sheet for verification. In some embodiments, at step 2140, the positions and Kit IDs are verified by a user. In some embodiments, after verification, the scanner will scan the information provided on the matrix sheet (on the opposite side of the samples). In some embodiments, at step 2150, the application saves a high quality image of the scan to a database.

In some embodiments, after verification of the Kit IDs, at step 2145 the samples are scanned and processed. In some embodiments, the matrix sheet must be flipped to scan the samples. In some embodiments, the samples and patient information are scanned simultaneously. In some embodiments, an identification tag on the same side of the sample and demarcation provides the patient information. In some embodiments, wherein multiple sheets are to be scanned and processed, a sheet button is selected (e.g., 2001 of FIG. 20A) and the process of scanning the samples and kit IDs is repeated.

3. Editing and/or Verification of Delineations

In some embodiments, as disclosed herein, the adhesive patches containing the samples comprise a demarcation. In some embodiment, processing of the scanned image comprises overlaying a polygon to mark the delineation on which the sample will be cut by a cutting device as described herein. In some embodiments, the overlay delineation is verified prior to cutting of the adhesive patches.

In some embodiments, at step 2155, the delineation overlay is edited prior to cutting of the sample collectors/adhesive patches. In some embodiments, editing tools (as depicted in FIG. 20C) are utilized to edit a polygon overlay which represents the delineation at which the adhesive patch will be cut. In some embodiments, an overlay polygon comprises one or more manipulation points. The manipulation points may be moved such that the overlay better matches a demarcation provided on the adhesive patch. Additional lines and/or curves may be added to the overlay, if the demarcation or computer-generated delineation is incomplete. Further, the computer-generated delineation may be resized, rotated, or portions of the delineation may be erased to better match the demarcation. In some embodiments, a delineation may be manually drawn using the software application. In some embodiments, incomplete or faded demarcations are filled in manually using a marker. In some embodiments, images of the original demarcation are scanned and saved to the database prior to manually completing the demarcation. In some embodiments, at step 2160, the delineations are verified prior to proceeding to the cutting procedure.

As described herein, the editing procedure may utilize one or more images of the actual lesion, mole, or skin area of interest captured using a user facing software application (see procedure depicted by FIGS. 19A-19Z). In some embodiments, images captured by the user facing software application are overlaid onto the scanned images of the patches for verification and/or editing of the delineations using the computer software. In some embodiments, the delineation is fully automated. In some embodiments, a fiducial provided on the adhesive patch is used to resize, rotate, and/or keystone images of the lesion captured by the software application. In some embodiments, images captured by the user facing software application are utilized to generate a computer-generated delineation and no demarcation using a marker is required.

4. Cutting Procedure

In some embodiments, the scanner and cutting apparatuses are integrated into a single device and the matrix sheets comprising the samples are left in place for the cutting procedure. In some embodiments, wherein the scanner and cutter are separate devices, at step 2165 the matrix sheets are transferred from the scanner to the cutter after completion of the delineation verification and/or editing procedure. In some embodiments, wherein a delineation representing an outline to be cut is automatically generated, the cutting procedure may take place immediately after the scanning procedure.

In some embodiments, the matrix sheets are placed onto a cutting stage of the cutting apparatus. In some embodiments, the cutting stage comprises alignment pins to be received by through holes in the matrix sheet to facilitate proper alignment. In some embodiments, a protective cover sheet is removed from the adhesive matrix prior to cutting of the samples.

In some embodiments, once all matrix sheets containing samples to be cut are in place, a “Laser Cut” button is selected on the GUI of the software application. In some embodiments, the application displays a new window allowing selection of a sheet, multiple sheets, or all sheets to be cut by the laser along the delineations. In some embodiments, upon confirmation of the sheet selection, at step 2170, the cutter will cut the adhesive patches along the delineations.

5. Post-Cut Procedure

In some embodiments, the software application generates a “pre” image and measurement data. In some embodiments, the software application will automatically create a new folder and save the images with a time and date stamp. In some embodiments, the saved files are named by a barcode read on the adhesive patch. In some embodiments, “pre” images will be saved with the suffix_Pre.jpg for the image and_Pre.txt for the measurements.

In some embodiments, at step 2175, the cut areas containing the sample tissue are then removed from the matrix sheets. The cut areas containing the tissue sample may be then processed and test. It is important that transfer of samples is completed in an organized manner to prevent misplacement of samples. In some embodiments, at step 2180, the sheets are then scanned again to obtain the post-cut pictures. The post-cut scanning setup will mirror the scanning procedure described above. In some embodiments, after the post-cut matrix sheets are in place and ready to be scanned, a “PostScan” image button will be selected. In some embodiments, a window will appear and a “PostScan Image” button is selected. In some embodiments, correlation of the Kit ID and Accession numbers are verified. In some embodiments, after verification a “Post Scan and Process” button is selected.

In some embodiments, if there is a second sheet, and the scanner has a 1 sheet capacity, the first sheet is removed and the second sheet is placed on the scanner. In some embodiments, the “Sheet 2” option is selected and correlation the Kit ID and Accession numbers are verified. In some embodiments, the “Post Scan and Process” button is selected again for the second sheet. In some embodiments, once all of the sheets have been scanned, at step 2185, the data and scanned images are saved to a database.

B. Exemplary Methods of Sample Collection

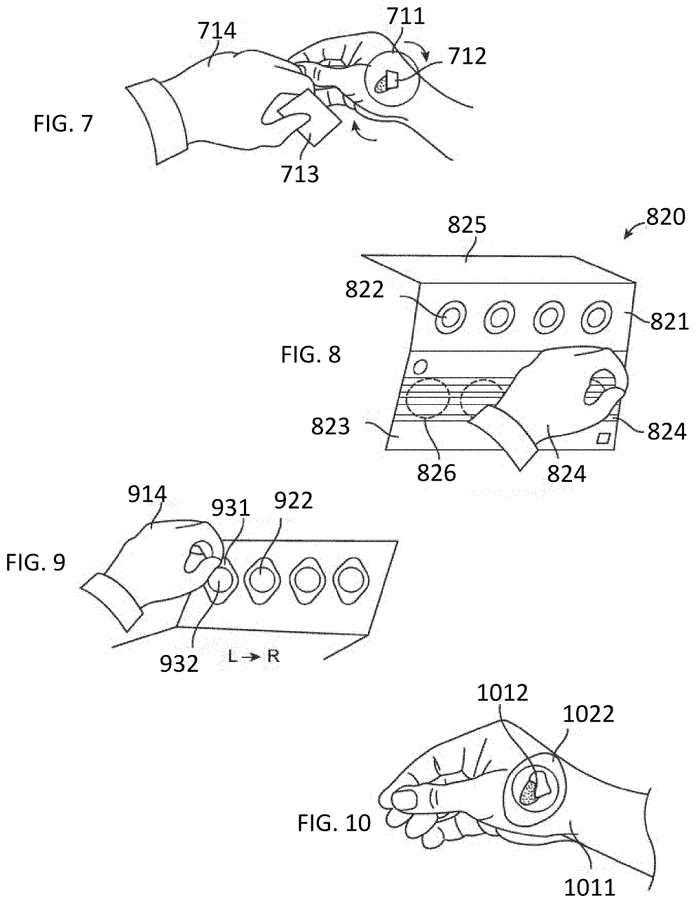

With reference to FIGS. 7-13, a skin sample collection system and method is depicted, according to some embodiments. In some embodiments, a pigmented lesion located on the hand of a subject is selected for skin sampling. The skin sampling area contains a minimal amount of hair, is not irritated and has not been previously biopsied. The lesion is about 8 mm in size. As exemplified in FIG. 7, the skin sampling area 711 comprising the skin lesion 712 is cleansed with an alcohol pad 713 by a practitioner 714 wearing gloves, and the skin is allowed to air dry for 5 minutes. The practitioner may be a patient of a self-administered sampling kit or a partner, caretaker, physician, or other professional assisting in the sampling of the patient.

FIG. 8 exemplifies the tri-fold skin sample collector 820 comprising a peelable release panel 821 comprising four adhesive tapes 822, a placement area panel 823 comprising a removable liner 824, and a clear panel 825. The tri-fold skin sample collector has a barcode specific for the subject. The removable liner is removed from the placement area panel 823, exposing four regions 826 designated for the placement of up to four used adhesive tapes. The four regions of the placement area panel are not exposed to any skin prior to application of a used tape.

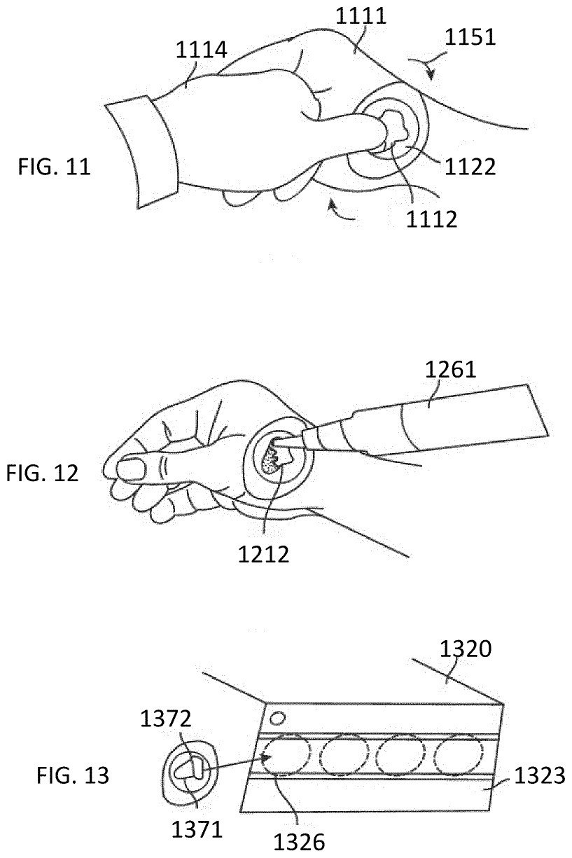

An adhesive tape is removed from the top left side of the peelable release panel as exemplified in FIG. 9. The practitioner 914 handles the adhesive tape 922 by the tab region 931 so that the matrix material of the central collection area 932 does not come in contact with a surface prior to skin application. The skin sampling area is held taut while the adhesive tape is applied onto the skin sampling area. An adhesive tape 1022 positioned on the cleansed skin sampling area 1011 comprising a skin lesion 1012 is exemplified in FIG. 10. The adhesive tape is pressed firmly on the skin while making circular motions. FIG. 11 exemplifies the practitioner 1114 pressing on the skin comprising a skin lesion 1112 while making a circular motion 1151. As exemplified in FIG. 12, the lesion area 1212 is demarcated on the adhesive tape 1222 using a marker 1261, which may be provided in a skin sample collection kit. The practitioner slowly removes the used adhesive tape from the skin sampling area by holding the tab and pulling in one direction. The used tape 1371 comprising a skin sample 1372 is placed on the first unoccupied skin collection region 1326 of the placement area panel 1323 on the tri-fold skin sample collector 1320 as exemplified in FIG. 13. The procedure is repeated with three additional tapes on the same lesion.

The tri-fold skin sample collector is folded and placed in a package provided with the skin sample collection kit. The package contains pre-paid postage and is self-addressed to a processing facility.

In an exemplary embodiment, a pigmented lesion located on the upper back of a subject is selected for skin sampling. The skin sampling area contains a minimal amount of hair, is not irritated and has not been previously biopsied. The lesion is about 15 mm in size. The lesion is sampled utilizing an adhesive skin sample collection kit. The skin sample collection kit includes an instructions for use sheet (or an instruction manual). The lesion is sampled by a capable person who has read and understood the skin sample collection kit instructions for use sheet.

A pair of gloves is removed from the skin sample collection kit and the fitted onto the person performing the skin sampling procedure. The skin sampling area comprising the pigmented lesion is cleansed with an alcohol pad provided in the adhesive skin sample collection kit and the skin is allowed to air dry.

A tri-fold skin sample collector is removed from the adhesive skin sample collection kit. The tri-fold skin sample collector comprises a peelable release panel comprising four adhesive tapes, a placement area panel comprising a removable liner, and a clear panel. The tri-fold skin sample collector has a barcode specific for the subject. The tri-fold skin sample collector further comprises an area configured for providing patient information. The tri-fold skin sample collector is labeled with the subject's name and identifying information. The removable liner is removed from the placement area panel, exposing four regions designated for the placement of up to four used adhesive tapes. The four regions of the placement area panel are not exposed to any skin prior to application of a used tape.

An adhesive tape is removed from the top left side of the peelable release panel. The adhesive tape is handled by the tab region so that the matrix material does not come in contact with a surface prior to skin application. The skin is held taut while the adhesive tape is applied onto the skin sampling area. The adhesive tape is pressed firmly on the skin while making 10 circular motions. The lesion area is demarcated on the adhesive tape using a marker provided in the adhesive skin sample collection kit. The used tape is slowly removed in one direction by pulling the tab away from the skin. The used tape is placed on the first unoccupied skin collection region of the tri-fold skin sample collector. The skin sample procedure is repeated with three additional tapes on the same skin lesion.

The tri-fold skin sample collector comprising 4 used adhesive tapes is folded and placed in the package provided with the adhesive skin sample collection kit. The package contains pre-paid postage and is self-addressed to a diagnostics facility.

C. Collection System

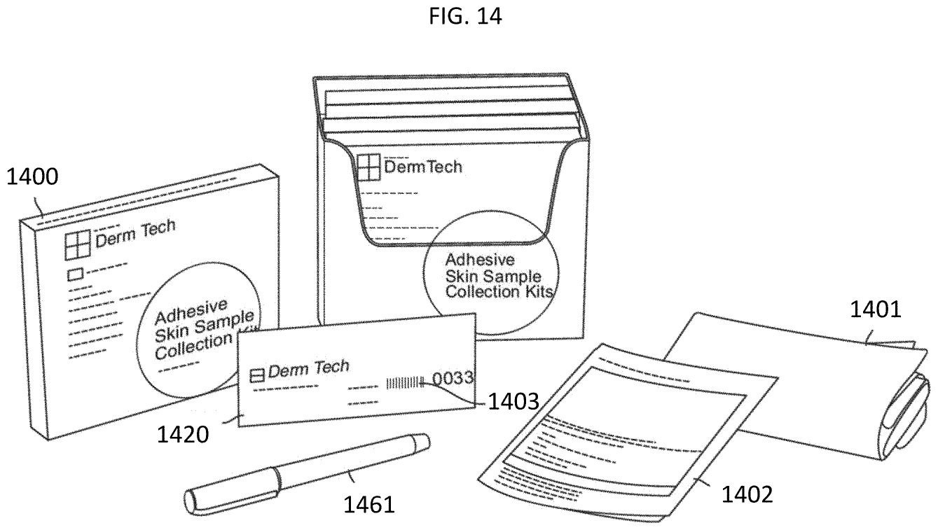

The adhesive skin sample collection kit components are stored in a cardboard box 1400 as exemplified in FIG. 14. The kit contains a tri-fold skin sample collector 1420 comprising four adhesive tapes, instructions for use sheet, a marking pen, a pre-paid, self-addressed shipping package 1401, and a shipping label 1402. The tri-fold skin sample collector comprises three panels including a peelable release panel comprising the four adhesive tapes, a placement area panel comprising a removable liner and a clear panel. The tri-fold skin sample collector further comprises a unique barcode 1403 configured to identify a subject. The adhesive tapes stored on the peelable release panel have an expiry date of 2 years from the date of manufacture. The skin sample collection kit is stored between 10° C. and 30° C. The instructions for use sheet (or instruction manual) include all information necessary to enable a person to understand and perform the method. The instructions for use sheet (or instruction manual) include diagrams describing steps of the skin sample collection method.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for isolating cells of interest from a tissue sample collection kit, the method comprising:
a) receiving one or more sample collectors comprising cells of interest;
b) positioning the one or more sample collectors on a substrate;
c) imaging the one or more sample collectors to generate at least one first image;
d) applying a software algorithm to the at least one first image to identify a delineation between the cells of interest and a surrounding portion of each sample collector, and
e) cutting the cells of interest from a remaining portion of each sample collector with a cutting system based on the identified delineation.

2. The method of claim 1, wherein the one or more sample collectors comprises one or more non-invasive, adhesive sample collectors.

3-4. (canceled)

5. The method of claim 1, wherein the one or more sample collectors comprises a plurality of sample collectors arranged in an array of sample collectors.

6-8. (canceled)

9. The method of claim 1, wherein imaging the one or more sample collectors to generate at least one first image is performed by an optical scanning system.

10. (canceled)

11. The method of claim 1, wherein the identified delineation comprises digital information comprising a plurality of points and one or more lines and/or one or more curves connecting the points to form an open or closed polygon.

12-13. (canceled)

14. The method of claim 1, wherein identifying the delineation between the cells of interest and a surrounding portion of a sample collector comprises delineating the cells of interest obtained from a lesion area from other cells obtained from a non-lesion surrounding area.

15. The method of claim 1, wherein the software algorithm comprises a computer vision algorithm.

16-18. (canceled)

19. The method of claim 1, wherein the cutting is performed with a mechanical cutting system, a plasma cutting system, or a laser cutting system.

20. The method of claim 19, wherein the cutting is performed with a laser cutting system.

21-22. (canceled)

23. The method of claim 1, wherein steps c)-e) are performed by an automated, computer-controlled apparatus.

24. The method of claim 23, wherein the automated apparatus has a throughput of at least 16, at least 32, or at least 48 sample collectors per minute.

25. (canceled)

26. The method of claim 1, further comprising analyzing the cells of interest.

27. A system for isolating cells of interest from a tissue sample collection kit, the system comprising:
a) an imaging apparatus;
b) a cutting apparatus; and
c) a computing device comprising at least one processor, a communications interface, and instructions executable by the last least one processor to provide an application;
wherein the computing device is communicatively coupled to the imaging apparatus and the cutting apparatus through the communications interface;
wherein the application is configured to perform operations comprising:
i. instructing the imaging apparatus to image one or more sample collectors to generate at least one first image;
ii. applying a software algorithm to the at least one first image to identify a delineation between the cells of interest and a surrounding portion of each sample collector; and
iii. instructing the cutting apparatus to cut the cells of interest from a remaining portion of each sample collector with a cutting system based on the identified delineation.

28. The system of claim 27, wherein the imaging apparatus comprises an optical scanner.

29. The system of claim 27, wherein the cutting apparatus comprises a mechanical cutting system, a plasma cutting system, or a laser cutting system.

30. The system of claim 29, wherein the cutting apparatus comprises a laser cutting system.

31-36. (canceled)

37. The system of claim 27, wherein the one or more sample collectors comprises one or more non-invasive, adhesive sample collectors.

38. The system of claim 37, wherein the tissue comprises skin tissue, and wherein the cells of interest comprise skin cells.

39. (canceled)

40. The system of claim 27, wherein the one or more sample collectors comprises a plurality of sample collectors arranged in an array of sample collectors.

41-42. (canceled)

43. The system of claim 27, wherein the identified delineation comprises digital information comprising a plurality of points and one or more lines and/or one or more curves connecting the points to form an open or closed polygon.

44-45. (canceled)

46. The system of claim 27, wherein identifying the delineation between the cells of interest and a surrounding portion of a sample collector comprises delineating the cells of interest obtained from a lesion area from other cells obtained from a non-lesion surrounding area.

47. The system of claim 27, wherein the software algorithm comprises a computer vision algorithm.

48-50. (canceled)

51. The system of claim 27, wherein the system has a throughput of at least 16, at least 32, or at least 48 sample collectors per minute.
